SEQUENCE LISTING (33 sequences)

SEQ ID NO: 1 - DNA, 20 bp, synthetic construct
  source 1..20: mol_type = other DNA
gcctaccgccataacttccg

SEQ ID NO: 2 - DNA, 20 bp, synthetic construct
  source 1..20: mol_type = other DNA
ctcaaattctgaagctcagt

SEQ ID NO: 3 - protein, 16 aa, synthetic construct
LAYRHNFRGCIENVIF

SEQ ID NO: 4 - protein, 16 aa, synthetic construct
LAYRHNFRGCIENVIY

SEQ ID NO: 5 - protein, 16 aa, synthetic construct
LPGKVNFRGCMENVFI

SEQ ID NO: 6 - protein, 16 aa, synthetic construct
LSHKQNFRGCLENILF

SEQ ID NO: 7 - protein, 19 aa, synthetic construct
TQVVIGDTNRSTSEAQFFL

SEQ ID NO: 8 - protein, 19 aa, synthetic construct
TQVVVGDTNRSNSEAQFFL

SEQ ID NO: 9 - protein, 19 aa, synthetic construct
TQVVIGDTNRSSSEAQFFL

SEQ ID NO: 10 - protein, 19 aa, synthetic construct
TQVVVGDTNRSSSEAQFFL

SEQ ID NO: 11 - protein, 19 aa, synthetic construct
RRLVIGDTNRTGSEAQYSV

SEQ ID NO: 12 - protein, 19 aa, synthetic construct
TRVVVGDTNRTGSEAWFSV

SEQ ID NO: 13 - DNA, 38 bp, synthetic construct
  source 1..38: mol_type = other DNA
gccataacttccgtggccgcatagaaaacgtgatctac

SEQ ID NO: 14 - DNA, 38 bp, synthetic construct
  source 1..38: mol_type = other DNA
gtagatcacgttttctatgcggccacggaagttatggc

SEQ ID NO: 15 - DNA, 33 bp, synthetic construct
  source 1..33: mol_type = other DNA
ggtaggtgatacaaactgctcaaattctgaagc

SEQ ID NO: 16 - DNA, 33 bp, synthetic construct
  source 1..33: mol_type = other DNA
gcttcagaatttgagcagtttgtatcacctacc

SEQ ID NO: 17 - RNA, 20 nt, synthetic construct
  source 1..20: mol_type = other RNA
gcctaccgccataattttcg

SEQ ID NO: 18 - DNA, 198 bp, synthetic construct
  source 1..198: mol_type = other DNA
cctaagcccatggaaatgtctcttcatctgcttcctacagatattcatcgggggtctagtgggcgcagcccgtaagaacctggcctaccgccataattttcgcggccgcatagaaaacgtgatctacaaccggatcaacattgcagaaatggcagtgatgcgccattcgcggatcacctttgaggccagtgggcaggg

SEQ ID NO: 19 - RNA, 20 nt, synthetic construct
  source 1..20: mol_type = other RNA
ctcaaattctgaagctcagt

SEQ ID NO: 20 - DNA, 198 bp, synthetic construct
  source 1..198: mol_type = other DNA
gagtcaccgaatcctatattcccacaggagaacagacaaggggctcctgacctttgtagaccatctgcctgtcactcaggtagtggtaggtgatacaaactgctcaaattctgaagctcagttcttcctgaggcctctgcgctgctatggtgaccgtgagtggcagactcctttggtgtgcctgtccagtactgtttc

SEQ ID NO: 21 - RNA, 20 nt, synthetic construct
  source 1..20: mol_type = other RNA
actccagtctttctagaaga

SEQ ID NO: 22 - DNA, 11 bp, synthetic construct
  source 1..11: mol_type = other DNA
ttagggtaatg

SEQ ID NO: 23 - DNA, 11 bp, synthetic construct
  source 1..11: mol_type = other DNA
ttaggttaatg

SEQ ID NO: 24 - DNA, 17463 bp, Homo sapiens
  source 1..17463: mol_type = genomic DNA
gaaaccggcgcgtgccaggagacagaggctggggaaggggggaggtgagaggaaagagggtggaaaggagaggatagagagagaagagcggaggaccaggaaccagagagagagagagagaaaagagagaggagagacagagcgcttgggggcgaaaggagagagggagggaagggtgggtaaggaggagagagcggtctgctgcaaaccccaggaggagagcttggagcccaagccagaactcgagccctagccggagccgttcacagggaggcggctgccgggaccgtcagccctgcatgatgcatctccggctcttctgcatcctgctcgccgcggtctcaggagccgagggctggggctactgtgagtgttgggcttggaggcaggtggggttgggcccaggagtccagagcctgcagggcggccccgaaccgcattgcggctgggtggtcgcgggtccttcccggccggcgcgcgcccgctggctctcatcttttcctgcctctcccccgcgctccgcattgcagctctgaggctgccgcgcgcgcccggggctggggctgggctctgggaagaggatgaagtcagcgggcagggatttggggttgggatccaagcaggagctggtgcctgggtttgaggctagggctgggggataagtgtatactggtgaccgatactaggattaaggttttgccagggcttaaggctggactttggagctggccaaggggtggggtttgtggctagggctggggtttgtggctggggctgggttttgtggcagggatccaagcaggagctggtgcccctgactgatgctaaggctggacatggagctggtgatggtgctagcactggctgaggttgggtgcaaggcctgtatttggggatggtgcaggttttgggggccgagttggattgaggctagggttggagctggattggtgtcttgggttgacttaggttgtggaagatgctgaggtttaggattgagtttgggaaagtactaaaccagggcaggtattgggctagctagggagggtctggccttgggcctataggtgtagcctaggggtttgcctagaaggagaaggtagcctcctctgagcatgcagctgttgctgcagccaggattgaatagatggctttaaggtagggctgggtgtgcctctgggggcgggggttgggggcacggaaggggaagcttcggtcgctaagtgggccggcctttggaagggtctgggaggggccagcgctgactaacagtcggtttccctaccctagacggctgcgacgaggagctggtgggtcccctgtatgcacgctccctgggcgcctcctcctactacagtctccttactgcgccgagattcgccaggctgcacggtgagctccgcggaacatcagctgccaactggcagcgcaccgcggaagggtgggggcctccggaggacttcggggagagggatagccggttaaagctcctgtcctttctataggcataagcgggtggtcaccacggattggggatccgaatccctggctccagatagacttaatgaagaagcaccggatccgggccgtggccacacagggctcctttaattcttgggactgggtcacacgttacatgctactctacggcgaccgagtggacagctggacaccgttctaccagcgagggcacaactcggtactctgggcgccaaggcggtaacacttggggagcttcctctgctccaggctctgggccgggcaccagcctctgggaaaatggagggggtggtggtgagggctcggacaaggagcagtgactccactccagggactctgtccagagggactgtcagcttaggacgtgcgcgaaacactcggttcacagggtttaacacactttagggtaaaacctgggagagcttcctaaggaggtgacatttgatttcaggtctgaggaaataataggattgtgggacagggcactttatggaaactgtgcgggcaaagccgccaagcagggaagaccaaggagtgtttactttccagaagtcgagattggctgggtgtgaaagttgtgggctgggaggctatggagaaagaagcagaggggggccgggcgcagtggctcacgcctgtaatcccagcactttgggaggccgaggcaggtggatcacgaggtcaggagttcatgaccagcctggccaacatggtgaaaccccgtctctatgaaaaatacaaaaaaatcagccgggcgtagtggcgggcgcctgtaatcccagctacttgggaggctgaggaagagaattgtttgaacccgggaggcagaggttgcggtgagccgagatcgtaccaccgcactccagcctgggcgacagagcgagactctgtctcaaaaaataaaataaaaaaaagaagcagagggcaggacgtggttactactctcctccacccccgcagaccttctttggtaacgtgaacgagtcggcggtggtgcgccatgacctgcacttccacttcactgcgcgctacatccgcatcgtgcccctggcctggaacccacgcggcaagatcggcctgaggctcggcctctatggctgcccatacagtaagtgtgcagagagcgcggagggggcctgggagacagcctccccagttcccggcccacctacggtcctttgcgcagaggccgacatactctatttcgacggcgacgatgccatctcctaccgcttcccgcgaggggtcagccgaagcctgtgggacgtgttcgccttcagcttcaagaccgaggagaaggacggtcttctgctgcacgccgagggcgcccagggcgactacgtgacgctcgagctggagggggcacacctgctgctgcacatgagcctgggtgagctcggcgaccatgtgcgatgcggagccaacccctgaagctctctcaccgccctcctcgtggcacctcctccgcgcatccgcgctcagcctggtcttccacttctctaaggcctggaccaaactgcccctttcttgtagcatttggtgctagcaaaacactggagttgagtgagaactggattccagtctcagctctaccactacacagagatgtgacctcggatggggcacttccgagcctcagttccctccttgtaaacacacacacacagacgcacacacattgtatctcattagcttttcataacacccgcacatgaaatagaagcagctcatgtgttcccagttttacagacaggaagccaggctaaatggcttaccctgtcacacactcgccaatggctgttgatctattcgcccactctccctgattgccctgggctttgttacacgctgctgctctgcctaggagcttggactccatggagttctcctggcttgaggtttcactctgtcctgccccaccctcaggcagcagccctatccagccaagaccaggtcacaccaccgtgagcgcaggcggagtcctcaatgaccagcactggcactatgtgcgggtggaccgatttggccgcgatgtaaatttcaccctggacggctatgtgcagcgctttattctcaatggagacttcgagaggctgaacctggacactgaggtgagagactagggaggtgctatttcgtggtagggtagatgctggatgagtgagtgcaggtcggtctctccctttctcttttcctctgtctctcagaggaaagacctggcattcaggctgtgggtggtggctcatgcctgtaatcccagcactttgggaggctgaggtgggaggattgcttgaggccaggtgttcaagactcacctgggcaacatagtgacatcctacctctatttaaaaaaattttttttttcaaagacctcgcattccaattcctgaatatcctagcagcccttggcaaacttgccactcaccagaaaaaggcctgttttttttttttttttttttttttgtgggtgttgttgctgttagcatgcagactcccaggcactatttcagaactaatgagatggggtttggaaattcagattttggggactaggcaaggtaggaaaattgttcccttggatcctcccacaactttgtggagttgttaaagaccctgaccccatctgcagataagaatcccaggcaggctaattgacttgcccaagggcgtgcagccgatttgtgttggagtcagagcttgaactcaagccttgaaaggcctagcgagctttagtaccgacactggggaaatgtgtgttttaagtcttttacaaaaccccatctggcatttgggaaagcatacggcggggacgcgcgagaaaggcaggcgccctcctgtgcccaagaggcctcattccccacgcctcccgcagatgttcatcggaggtctggtgggcgccgcgcggaagaacctggcctatcggcataacttccgcggctgcatagaaaacgtaatcttcaaccgcgtcaacatcgcagacctggccgtgcggcgccattcccggatcaccttcgaggccagtgggcaggggggtctgggaggacaggatatcaaagcgtcgtggaaagcaaagaggtggagcgggaagagatattgaacatcagataaaagcggagaatccctctgtccccagccagatgctcaagttgggaggggagcgggtctcacctgaggtgcatgagccacgcaggcccctagttaagaagcagtggtcgggtccaatcaccttcttagttcatagatgaagaaagtaaggcgcagacacagggagtggcttgtccaggggtcgtgcagctgcacggtggctgagtagggacttgaaccgatggcttctctgatatgcccccctcaaccctctccgactctggagctctgttgggaagctggcaggagccaggtctgtggtccaaaattgcctctcgatactggaaggagagaagcggtcgaatcgcagacggtggagatcagcgagagcaagcgagcagagttccgagggccgactgggttggggccccctccacagatggacgagcttggaggggaagaggtcacgtcatggttggagatctcacccccgccaacaccgaaggagggcggtgaccagggtcttagaccggtgtgaaaactgaattcccagctgaggcagaggcggctcacgggtgttgatgcgtcttcacttttgcccctagggtaaggtggcttttcgttgcctggacccggtaccgcaccctatcaacttcggaggccctcacaacttcgttcaagtgcccggtttcccacgccgtggccgcctggcagtctcatttcgcttccgcacctgggacctcaccgggcttctccttttctcccgtctgggggacgggctgggccacgtggagctgacgctcagcgaagggcaggtcaacgtgtccatcgcgcagagcggccgaaagaagcttcagttcgctgctggtgagggcgtttcgggggaggcacaagaagagaagagaagtgtagaggatcccaggaaagttgtaggcctggactgaggcctttacattctggagaggggagaggagtgaagggggcagggcaggaggccccagggtactgggaaaaggtgggagtggctagaggcagtgaggaggcgaatctgagggactaagtattcttggagaagggggaagcctgggagtcctggtattctccagttccagaaggcccctggagatttggttactcgtttataactgtgcagactctgtggcagttgctgggtcatggataatgaagagacattcagagaggtctccactgggtgtgagaagtgtaatcacgggggcacacaggctgctactgggacacagtggggctgctgcttccccactcagaacattcttctaccctagttgctgcttccctccacccacaccatcatccattcttgtccagggtaccgactgaatgacggcttttggcacgaggtgaattttgtggcacaggaaaaccatgcagttatcagcattgatgatgtggaaggggcagaggtcagggtctcatacccgttgctgatccggacagggacctcatatttctttgggggtaagtgggggccaacctgaccagacctctgtttctgggtgggaaggctccatctaagtccacccagatggggccacatggagaattttggagggatcaagccctcttcccctctggggcctcagggagtggaaggtcattgccatctacactttggttgtagtcccctttcttcctttatgtctggctccccctcagcatgtccctgggggagggtctttggggtctcccctggggaggatctcacaggggtggttgctccaggttgtcccaagccagccagtcgatgggactgccactccaaccagacggcattccatggctgcatggagctgctcaaggtggatggtcaactggtcaacctgactctggtggagggccggcggcttggattctatgctgaggtcctctttgatacatgtggcatcactgataggtacccagaagcccctaacaagagatgacccctccaaagccctcttccctggtctcccagtaggaatggcctctttcaataatctcccttctccttgtctctgctccttagtcccctgcttggggatgattcttctttgatctcttacctcctatttcttcccttaaggtgatacatgctcaggatggaaatgacacatttgcttttggtcctagaggggttttttgtgctagggggatgataggggtagaataaggcatgttttgatgttttcaggaagggggaatagtagtacttgagccctgaggactgcctagtgcccctgcatctgcgcttatacccggctggctaggcggggtgtgtctcaccgggttctggggcttcaaggagctgggagtgaagcttgcagggatcagaccccactctccagctcccagtaaggctccttcccttggtcctgaccccttccctaggtgcagccctaacatgtgtgagcatgatggacgctgctaccagtcttgggatgacttcatttgctactgcgaactgacgggctacaagggagagacctgccacacacgtaagccagatgtggtatggggggagtcagggacaagggaggtcaatagaaggttgctggggatcatcaggctgctagagatggcgaggatggcccttccagccctctcgctttttgtttttggtttttttgagacagagtcttgctctgttgcccaggctagagtgcagtggtgccatctgggctcgctgcaacctctgcctcctgggttcaagcgattctcctgcctcagcctcctaagtagctgggactacaggtgcacaccaccacgcctagctaatttttatatttttattagagatggggtttcaccatgttggccaggctggtctcgaactcctgacctcgggtgatgcaccaccccggcctcccaaagtgctgggattacaggcccgagccgccgcacctggccagccctctaactctctaactgcctttgtctcaacctattatctgccagctttgtataaggaatcctgtgaggcttatcggctcagtgggaaaacttctggaaacttcaccattgatcctgatggcagtggccccctgaagccatttgtagtgtactgtgatatccgaggtaagtgtctctgttgggtggtgagggggtgaggggagaaccaggaaggatagagtggtgggtgggggccagttagactaaacaagtgagggtaaaggaggacagaggggaagggcatactgaggaatttgtaacctagccttaaaagtaaatcccaagccaggcgtggtggctcacacctgtaatcccagcactttgggaagctactcgggaggttgaggcaggagaatcacttgaacctgggaggtggaggttgcagtgagctgagatagcgccattggactccagcctgggcaacagagcgagacttcatctcaaaaaaaaaaaaaaaaaaagtatatcccagcttccttacccattccagctatcttatttccagcctacaatgcagagtgcccagggcagaggtggaatggagctgctggccacgttcagtggctctgagttgcagcagcggtggtggaggtgggcagggagatgggtagagaatgcccaactccagccaaagctctgtcccctcttgtctgccagagaaccgagcgtggacagttgtgcggcatgacaggctgtggacaactcgagtgacaggttccagcatggagcggccattcctgggggctatccagtactggaatgcatcctgggaggaagtcagtgcccttgccaatgcttcccagcattgtgaacagtggatcgagttctcctgctacaattcccggctgctcaacactgcaggttagggctggggtcagggaggtggcggaactggaggagacaccaatgggggcctgggagaaggaagccagagagccagctggggccttgggttggaagattcaaggaggggtctgaactcgctggaagggcgagaggagcccagaggctaatggagggacagggcctggaagctgcctgcacctcttccccaggaggctacccctacagcttttggattggccgaaatgaggagcagcacttctactggggaggctcccagcctgggatccagcgctgtgcctgtggtctggaccggagctgtgtggaccctgccttgtactgcaactgtgacgctgaccagccccagtggtgagggggcaaagggacagggtttttaggactccgggagtgggaggagctgagtggctggggctgagccatgacatcctgcccccacaggagaactgacaagggactgctgacctttgtggaccatctgcctgtcactcaggtagtgataggggatacgaaccgctccacttctgaggcccagttcttcctgaggcctctgcgctgctatggcgatcgtgagtggcagtccccttttgtgtgccctcccagagctgactctccagtttccaaaatctaggccgcatgtcactggtggtctctagctggggtgcccgacccccagctcctgctgtgatgcgatctcattacccctctgcacctggctcctctttcattccactccttagtctcccctccgtcacctcaaaccctccccctttgcccaaccttccctgcccccaaccccaggttctcttcctcctccaccctccaatctccctgacaagaccttcctccatctgcttttctaggaaattcctggaacaccatttccttccacaccggggctgcactacgcttccccccaatccgtgccaaccacagcctggatgtctccttctacttcaggacctctgctccctcgggggtcttcctagagaatatggggggcccttactgccagtggcgccgaccttatgtgcgggtggaactcaacagtgagcaggcagactgtgggagggcctcggggtagatgaaagtgctgtctggggagacaggggtaggctggggttggagccaagggcagatgcccttttgacaggcagcatgggaagggttgagataccctatgttctagccctcactcggtcttgacggctggctgggtgaccctaggcattcacttggcctctcttggccttaagccccttccttgtgccataaggaagctggaccttcgagtccctctcaccgctgattagccatgcttctagcctgggaagtgaggctgggaagctggcttcagggatctgtgatgtggggagagacaaagggtatgaggtttttaagcagctggtaagtttcaatgcaagctacagacaaattggagggatattcaagaagctccaaagaggtgagggaaagaggttatattgggcaaggatctatgaagggtcttgtaggtctgagtccttttctcccctaccctgcatcacactgtccagcatcccgggatgtggtcttcgcctttgatgtggggaatggggatgagaacctcacagtacactcagacgactttgagttcaatgatgacgagtggcacctggtccgggctgaaatcaacgtgaagcaggcccggctccgagtggatcaccggccctgggttctgcggcctatgccactgcagacctacatctggatggagtatgaccagcccctctatgtgggtaagcagcaacccagaggcaagtctgaagcctcctttacctccccatcctccaaggccactcgccttgtttccaggagcctcctgtcttaggtgccagtcccctctgcttcagagcaagccagtgtctcacttgtccctccaggcctttgtatcctattctattcttccagccctcttggctccctttccaactacaatacttcatcctgtctctggctctgcctccatttcttttcttttttttttttttttgagacggagtctcgctctgtcacccaggctggagtgcagtggcacgatctcggcccactgcaagctccgcctcctgggttcaagcagttctcctgcctcagcctcccgagtagctgggactacaggcgcccgccaccgtgcccagctaattttttttgtattttttagtagagacggggtttcatcatgttagccaggatggtctcgatctcctgagctcgtgattcgcccgcctcggcctcccaaagtgctgggattacaggcgtgagccaccgcgcctggccctgcctccatttcttgacctgttgcctacccttcccaggatctgcagagcttaagagacgcccctttgtgggttgcttgagggccatgcgtctgaacggagtgactctgaacctggagggccgtgccaatgcctctgagggtacctcacccaactgcacaggccactgtgcccaccctcggctcccctgtttccatggaggccgctgcgtggagcgctatagctactacacgtgtgactgtgacctcacggcttttgatgggccatactgcaaccacggtaagtgctgctggttatggggcaacagggagccatagggtgtaatgggatgtagggagggcagggaaggaaatagcatgtaaagcccatatcatggaaaattagagttgtccctggattcctgagctctgagtttggggagttaggcagttgaagatcatgggtgactccaaaggcatggacaaggaagggatggtacagatagctggtgcaaaaatatctcaaaatcaccaagtatgctttgggagcccaaggtgggaggattgcttgaggctaggagttcgagaccagcctgggcaacatggcaagaccctgtctctacaaaaaaaattctttttttttttatgagatggaatctcgctctgtcacccaggctagagtacagtggcacgatctcggctcactgcaacctccgcctggttcaagcgattcttttgcctcagcctcccgagtagctaggactacagatgtgcaccaccatgcctggctaatttttgtatttttagtggagatggggtttcaccacattggccaggctggtctcgaactcttgaccttgtgatctgcctgcctcagactcccaaagtgctgggattacaggcgtgagccaccgcgtctggccaaaataatttcttttttcttttttttttttgagactgagtttcactcttgttgcccaggctggagtgtaatgactcgatctcggctcactgcaacctccacctcccgggctgaagcgattctcctgcctcagccttccaagtagctgggattacaagtgtgtgccaccacacccggctaatttttgtatttttagtagagatggggtttcaccacgcttgccaggctggtcacgaacttctgaccgcaggtgatccacccaccttggcctcccaaagtgctgggattacagacgtgagccacagcgcctggcaaaattttttaattagctgggcatggtggtgcacacttaggcctagctactcgggaggctgaggtgggaggatcacttgcacctaggaggtcaaggctgcagtgagctatgatcgtgccactgcactccagcctggggggcacagcaaggactgtgtctacaaaaaaaaaaaaagcaaatcgcttctccatcttcccaccatcaccatggtaacacaggcgaactcccttgtcaatgtatttgatgattgctgcattcctaaaacatgtcaatgacccagttcttggttaaggtgagccttgacattccccatagcaagatcctacacacatgccaagactgcatcctccctaaggctggcctatagggaggatatatgagcaggtgggttactggtgggaggagtgtgggggactagggagatacaagacttagggaccaacagatttctggtggtggtttggaagtgtgagtagtggcacatatactacatggggtcaggatatccccatcacccaggctggagtgcagtggcatgatcttggctcactgcaacctctgccccctgggctcaagcaattctcctgccttggctttcctatagtagctgggattacaggcatgcactaccacacctagctaagttttgtatttttagtagagatggggttttgccatgttggccaggctgttctcgaactcctgacctcaagatgatctgcctgccttggcctcccaaagtgctgggattacaggtgtgagccactgtgcctggccttactgcgtgtctttaagcaagtcactactcatctctgagcctgtttccccatttgtaaaatgcaggggttggatcagatgatctccagtttcccatggctgtaagattctgttctgtgacagaggggctcaaggggacctaaaagtaaatggagggttgtgagggtggcagaaactgaagtaagtctcaggatgtgtgtatggatgacataaaacctctgaattggggagtgagcagtgtgggggccctaacctccctgcttctacctgcagatattggtggtttctttgagccgggcacctggatgcgctataacctacagtcagcgctgcgctctgcagccagggagttctcccacatgctgagccggccagtgccaggctatgagcctggctacatcccgggctatgatactccgggctatgtgcctggctaccatggccccgggtaccgcctgcccgactacccccggcctggtcggcctgtgcccggttaccgtgggcctgtctacaacgttacgggagaggaggtctccttcagcttcagcaccagctccgcccctgctgtcctgctctacgtcagttcctttgttcgtgactacatggctgtgctcatcaaggatgatggtaagctctcccgggctctctcaccccactccagcttcaccccggtgcccctaattctcctattgattcacaaatacaaggacccacgtgctgttagaagataggagtcatggggcctgagaccccaaatttcttctccccaccccacagggacccttcagctgcgatatcagctgggcaccagtccctacgtgtaccagctaaccactcgaccagtgaccgatggccagccccatagcatcaatatcacccgtgtttaccggaacctcttcatccaggtatgcatagagggaggtgagccagttcagatcataacctcatggtctccgttgtggcatccaggaaaacatcaaatacttaatatttgtagatcacatttgaatgtatagaggattttcacgtgtgtcacctcatgtaaccctcacaatagctccaaggggacaataggcattttttttttttttttgagatggaattttgctcttgttgcccaggctggagtgcaatggtgtgatctcagctcactgcaacctctgcctcccgggttcaagcaattcttctgcctcagcctcccgagcagctgagattacaggcatgcgccacaacgcccagctaattttgtatttttagtagagacaggagttctccatgttggtcaggctggtctcgaacacccgacctcagatgatccgcctacctcagcctcccaaagtgctgggattataagcgtgagccaccgcgcccagccagacaataggcattcttatttaagtgttttacaaatcagtaaagagcctcagaaagtaaagcggtttcagctactcaggagggtgaggcaggaaaattgcttgaacctgggaggtggaggttgcagtgagctgagatcgtgccactgcactccaggcctaggcgacagagtgagactccatctcaaaaaaataaagtggtttacccaaagtcaaaccgctggtaagctgcaaagatggggcttaaaaccagggttttaaaaaatatataggccaggtgcagcggctcacacctgtaatcccaacactttgggaggctgaagtgggaggactgcttgaggccaggagtttgagaccaacctgagcaacatagcaagacctcatcttcacaaagaaaaatatttaataaataaaataaaataataataataaaaatatatataccaaatcctacatctaggcccaggggttgcccacttcagattgagctaggactcacccagctggcaccgccagtttggacagaatgagagatctccagtatctgcccccaagtatattcccagggtccacacagttccagtccagcccacaggcatctgcttctggtccccgctccctcatcgccctccccctccccctccccacctcaggtggactacttcccactgacagagcagaagttctcgctgttggtggacagccagttggactcacccaaggccttgtatttagggcgtgtgatgggtaagctgcgggtgcggacgcgttttaggcaaggagctgtggcatatgttcctggatcctcaaggaaagtgaaggccctgggaatggctgagggcagtgggaaggatgagtgggaaacctgtggacagtgagagtggcagggcctttgggtccaagtccctctttctgggcctgcctctctctcagagacaggagtcattgacccggagatccagcgctacaacaccccaggtttctcaggctgcctgtctggtgttcgattcaacaacgtggctcccctcaagacccacttccgaacccctcgacccatgactgctgagctagctgaggcccttcgagttcagggagaactgtccgaatctaattgcggagctatgccacgtcttgtttcagaggtgccacctgagcttgatccctggtatctgcccccaggtacattcccaggacacagaggacagaagggagggatgacacggaagggaatgattcttaacatggaggaggcatctcctaactggtgctttctcctctccagacttcccctactaccatgatgaaggatgggttgccatacttttaggctgtgagtagcactgatcactaagctgacctcccaagactaccctctgactgtcagcatcctttccctgcccctgatccccaaggctgcaggatggcaaaggcactagatgcacaatggcacaaaggatgatgagacaggctatgctgaaggtgccatataactgggcagagatttttagatgtttgaggatgagacaggctatgctgaaggtgacatataactgggcagagatttttagatgtttgaggatgctacagatttttaaataaaatattaaaatgagactatagctaagggatgggaaaatggggatacagtaacacctagagagataagaaattggggtaactaccagggatgtggaaagagaatattagaaacagaaggtggcagggggtaagccaggaattcaaacctgccagacacttgagatgagtttagtgtggtcgaacagctaactgagggtcatacattatgcttttggccaaaagtaaatgtgtgtgtgtgtgtgtgtgtgtgtgtgtgtgtgtgcaggtgaaatcccaaagtgtgtgtatgtatacaggtgaaatctcaaagagtgcgtgtgtgtgtgtgtgtgtgtgtgtgtgtgtgtgtatacaggtgagatcccaaagatctgaattgtcccttttcttcttttcagttttggtggcctttctgctgctggggctggtgggaatgttggtgctcttctatctgcaaaatcatcgctataagggctcctaccataccaatgagcccaaggctgcccacgagtaccatcctggcagcaaacctcccctacccacttcaggccctgcccaggtccccacccctacagcagctcccaaccaagctccagcctcagccccagccccagccccaactccagccccagcccctggcccccgggatcagaacctaccccagatcctggaggagtccaggtctgaatgagtcagaagggcttctgggaccaattccagctcctgacattcccccagtcctgcctctcccccatcctatcagggacatttggctcctcttagctggctctgctcatccagaggatattcccccatcccccccccatcaagtttggtgggcagagctacagatgggacccaagggagtggccgagcctcactgcctaaaccaatgcccttctcatccctgtttccccaggctcctggctgtttatctgccccaaaggagaagcctcatggggttgacataggtcctttctgccatctctgttccagctgctgtcagggattaacaacagagtgtaggggagattaactgcctcccttccaatagacactatcagcagggacagatgtgtgggagtgcagggctgcagagggtatggggggaggaggctgctaaaccctatcccccagcctcccccctgccctgaagatcttccatttgcttccactcagctggaggctcaagagggcttgatggctgtcccctgcccccctccttttgttttgtacacagagaccaagaggcctcagtttagcaccttagtacctccgctgcttcacttgctttagccaaagccataaaaaacctgcaacgtagagaaaataatgcagataccctgactagccagccctctactcctccaaccttttccaagatatgcaatggcctttgtgcctgcccaaaggcttcgccccctccagtgcatgaggaaccctctttcctccgctcagagatgctgcttcatttacccaggaggtcatattctttatatatattttttgttgcaaagtgtctctctagagaaactctatatattattcgaatttttaaattatttgtttatatataaaagaaaagctcaattggctgtgctgtcctgtgctgtgctgtgcccacagtctgtagtttgcctttcctgtgttggaagtgtcgtgagtcctgctggtcacccaccagcctggtccggcctccactctgaagcagggttgagcctgctgtgctctcagacgcggagccgttccaataaagcagagtggcagagcccca

SEQ ID NO: 25 - protein, 1384 aa, Mus musculus
MMSLRLFSILLATVVSGAWGWGYYGCNEELVGPLYARSLGASSYYGLFTTARFARLHGISGWSPRIGDPNPWLQIDLMKKHRIRAVATQGAFNSWDWVTRYMLLYGDRVDSWTPFYQKGHNATFFGNVNDSAVVRHDLHYHFTARYIRIVPLAWNPRGKIGLRLGIYGCPYTSSILYFDGDDAISYRFQRGASQSLWDVFAFSFKTEEKDGLLLHTEGSQGDYVTLELQGAHLLLHMSLGSSPIQPRPGHTTVSLGGVLNDLSWHYVRVDRYGRDANFTLDGYAHHFVLNGDFERLNLENEIFIGGLVGAARKNLAYRHNFRGCIENVIYNRINIAEMAVMRHSRITFEGNVAFRCLDPVPHPINFGGPHNFVQVPGFPRRGRLAVSFRFRTWDLTGLLLFSHLGDGLGHVELMLSEGQVNVSIAQTGRKKLQFAAGYRLNDGFWHEVNFVAQENHAVISIDDVEGAEVRVSYPLLIRTGTSYFFGGCPKPASRWGCHSNQTAFHGCMELLKVDGQLVNLTLVEFRKLGYFAEVLFDTCGITDRCSPNMCEHDGRCYQSWDDFICYCELTGYKGVTCHEPLYKESCEAYRLSGKYSGNYTIDPDGSGPLKPFVVYCDIRENRAWTVVRHDRLWTTRVTGSSMDRPFLGAIQYWNASWEEVSALANASQHCEQWIEFSCYNSRLLNTAGGYPYSFWIGRNEEQHFYWGGSQPGIQRCACGLDQSCVDPALHCNCDADQPQWRTDKGLLTFVDHLPVTQVVVGDTNRSNSEAQFFLRPLRCYGDRNSWNTISFHTGAALRFPPIRANHSLDVSFYFRTSAPSGVFLENMGGPFCRWRRPYVRVELNTSRDVVFAFDIGNGDENLTVHSDDFEFNDDEWHLVRAEINVKQARLRVDHRPWVLRPMPLQTYIWLVYDQPLYVGSAELKRRPFVGCLRAMRLNGVTLNLEGRANASEGTFPNCTGHCTHPRFPCFHGGRCVERYSYYTCDCDLTAFDGPYCNHDIGGFFETGTWMRYNLQSALRSAAREFSHMLSRPVPGYEPGYVPGYDTPGYVPGYHGPGYRLPEYPRPGRPVPGYRGPVYNVTGEEVSFSFSTNSAPAVLLYVSSFVRDYMAVLIKEDGTLQLRYQLGTSPYVYQLTTRPVTDGQPHSVNITRVYRNLFIQVDYFPLTQKFSLLVDSQLDSPKALYLGRVMETGVIDPEIQRYNTPGFSGCLSGVRFNNVAPLKTHFRTPRPMTAELAEAMRVQGELSESNCGAMPRLVSEVPPELDPWYLPPDFPYYHDDGWIAILLGFLVAFLLLGLVGMLVLFYLQNHRYKGSYHTNEPKATHDSHPGGKAPLPPSGPAQAPAPTPAPTQLPTPAPAPAPAPASGPGPRDQNLPQILEESRSE

SEQ ID NO: 26 - protein, 1385 aa, synthetic construct
MMSLRLFSILLATVVSGAWGWGYYGCNEELVGPLYARSLGASSYYGLFTTARFARLHGISGWSPRIGDPNPWLQIDLMKKHRIRAVATQGAFNSWDWVTRYMLLYGDRVDSWTPFYQKGHNATFFGNVNDSAVVRHDLHYHFTARYIRIVPLAWNPRGKIGLRLGIYGCPYTSSILYFDGDDAISYRFQRGASQSLWDVFAFSFKTEEKDGLLLHTEGSQGDYVTLELQGAHLLLHMSLGSSPIQPRPGHTTVSLGGVLNDLSWHYVRVDRYGRDANFTLDGYAHHFVLNGDFERLNLENEIFIGGLVGAARKNLAYRHNFRGRIENVIYNRINIAEMAVMRHSRITFEGNVAFRCLDPVPHPINFGGPHNFVQVPGFPRRGRLAVSFRFRTWDLTGLLLFSHLGDGLGHVELMLSEGQVNVSIAQTGRKKLQFAAGYRLNDGFWHEVNFVAQENHAVISIDDVEGAEVRVSYPLLIRTGTSYFFGGCPKPASRWGCHSNQTAFHGCMELLKVDGQLVNLTLVEFRKLGYFAEVLFDTCGITDRCSPNMCEHDGRCYQSWDDFICYCELTGYKGVTCHEPLYKESCEAYRLSGKYSGNYTIDPDGSGPLKPFVVYCDIRENRAWTVVRHDRLWTTRVTGSSMDRPFLGAIQYWNASWEEVSALANASQHCEQWIEFSCYNSRLLNTAGGYPYSFWIGRNEEQHFYWGGSQPGIQRCACGLDQSCVDPALHCNCDADQPQWRTDKGLLTFVDHLPVTQVVVGDTNRSNSEAQFFLRPLRCYGDRNSWNTISFHTGAALRFPPIRANHSLDVSFYFRTSAPSGVFLENMGGPFCRWRRPYVRVELNTSRDVVFAFDIGNGDENLTVHSDDFEFNDDEWHLVRAEINVKQARLRVDHRPWVLRPMPLQTYIWLVYDQPLYVGSAELKRRPFVGCLRAMRLNGVTLNLEGRANASEGTFPNCTGHCTHPRFPCFHGGRCVERYSYYTCDCDLTAFDGPYCNHDIGGFFETGTWMRYNLQSALRSAAREFSHMLSRPVPGYEPGYVPGYDTPGYVPGYHGPGYRLPEYPRPGRPVPGYRGPVYNVTGEEVSFSFSTNSAPAVLLYVSSFVRDYMAVLIKEDGTLQLRYQLGTSPYVYQLTTRPVTDGQPHSVNITRVYRNLFIQVDYFPLTEQKFSLLVDSQLDSPKALYLGRVMETGVIDPEIQRYNTPGFSGCLSGVRFNNVAPLKTHFRTPRPMTAELAEAMRVQGELSESNCGAMPRLVSEVPPELDPWYLPPDFPYYHDDGWIAILLGFLVAFLLLGLVGMLVLFYLQNHRYKGSYHTNEPKATHDSHPGGKAPLPPSGPAQAPAPTPAPTQLPTPAPAPAPAPASGPGPRDQNLPQILEESRSE

SEQ ID NO: 27 - protein, 1385 aa, synthetic construct
MMSLRLFSILLATVVSGAWGWGYYGCNEELVGPLYARSLGASSYYGLFTTARFARLHGISGWSPRIGDPNPWLQIDLMKKHRIRAVATQGAFNSWDWVTRYMLLYGDRVDSWTPFYQKGHNATFFGNVNDSAVVRHDLHYHFTARYIRIVPLAWNPRGKIGLRLGIYGCPYTSSILYFDGDDAISYRFQRGASQSLWDVFAFSFKTEEKDGLLLHTEGSQGDYVTLELQGAHLLLHMSLGSSPIQPRPGHTTVSLGGVLNDLSWHYVRVDRYGRDANFTLDGYAHHFVLNGDFERLNLENEIFIGGLVGAARKNLAYRHNFRGCIENVIYNRINIAEMAVMRHSRITFEVNVAFRCLDPVPHPINFGGPHNFVQVPGFPRRGRLAVSFRFRTWDLTGLLLFSHLGDGLGHVELMLSEGQVNVSIAQTGRKKLQFAAGYRLNDGFWHEVNFVAQENHAVISIDDVEGAEVRVSYPLLIRTGTSYFFGGCPKPASRWGCHSNQTAFHGCMELLKVDGQLVNLTLVEFRKLGYFAEVLFDTCGITDRCSPNMCEHDGRCYQSWDDFICYCELTGYKGVTCHEPLYKESCEAYRLSGKYSGNYTIDPDGSGPLKPFVVYCDIRENRAWTVVRHDRLWTTRVTGSSMDRPFLGAIQYWNASWEEVSALANASQHCEQWIEFSCYNSRLLNTAGGYPYSFWIGRNEEQHFYWGGSQPGIQRCACGLDQSCVDPALHCNCDADQPQWRTDKGLLTFVDHLPVTQVVVGDTNRSNSEAQFFLRPLRCYGDRNSWNTISFHTGAALRFPPIRANHSLDVSFYFRTSAPSGVFLENMGGPFCRWRRPYVRVELNTSRDVVFAFDIGNGDENLTVHSDDFEFNDDEWHLVRAEINVKQARLRVDHRPWVLRPMPLQTYIWLVYDQPLYVGSAELKRRPFVGCLRAMRLNGVTLNLEGRANASEGTFPNCTGHCTHPRFPCFHGGRCVERYSYYTCDCDLTAFDGPYCNHDIGGFFETGTWMRYNLQSALRSAAREFSHMLSRPVPGYEPGYVPGYDTPGYVPGYHGPGYRLPEYPRPGRPVPGYRGPVYNVTGEEVSFSFSTNSAPAVLLYVSSFVRDYMAVLIKEDGTLQLRYQLGTSPYVYQLTTRPVTDGQPHSVNITRVYRNLFIQVDYFPLTEQKFSLLVDSQLDSPKALYLGRVMETGVIDPEIQRYNTPGFSGCLSGVRFNNVAPLKTHFRTPRPMTAELAEAMRVQGELSESNCGAMPRLVSEVPPELDPWYLPPDFPYYHDDGWIAILLGFLVAFLLLGLVGMLVLFYLQNHRYKGSYHTNEPKATHDSHPGGKAPLPPSGPAQAPAPTPAPTQLPTPAPAPAPAPASGPGPRDQNLPQILEESRSE

SEQ ID NO: 28 - protein, 1385 aa, synthetic construct
MMSLRLFSILLATVVSGAWGWGYYGCNEELVGPLYARSLGASSYYGLFTTARFARLHGISGWSPRIGDPNPWLQIDLMKKHRIRAVATQGAFNSWDWVTRYMLLYGDRVDSWTPFYQKGHNATFFGNVNDSAVVRHDLHYHFTARYIRIVPLAWNPRGKIGLRLGIYGCPYTSSILYFDGDDAISYRFQRGASQSLWDVFAFSFKTEEKDGLLLHTEGSQGDYVTLELQGAHLLLHMSLGSSPIQPRPGHTTVSLGGVLNDLSWHYVRVDRYGRDANFTLDGYAHHFVLNGDFERLNLENEIFIGGLVGAARKNLAYRHNFRGCIENVIYNRINIAEMAVMRHSRITFEGNVAFRCLDPVPHPINFGGPHNFVQVPGFPRRGRLAVSFRFRTWDLTGLLLFSHLGDGLGHVELMLSEGQVNVSIAQTGRKKLQFAAGYRLNDGFWHEVNFVAQENHAVISIDDVEGAEVRVSYPLLIRTGTSYFFGGCPKPASRWGCHSNQTAFHGCMELLKVDGQLVNLTLVEFRKLGYFAEVLFDTCGITDRCSPNMCEHDGRCYQSWDDFICYCELTGYKGVTCHEPLYKESCEAYRLSGKYSGNYTIDPDGSGPLKPFVVYCDIRENRAWTVVRHDRLWTTRVTGSSMDRPFLGAIQYWNASWEEVSALANASQHCEQWIEFSCYNSRLLNTAGGYPYSFWIGRNEEQHFYWGGSQPGIQRCACGLDQSCVDPALHCNCDADQPQWRTDKGLLTFVDHLPVTQVVVGDTNCSNSEAQFFLRPLRCYGDRNSWNTISFHTGAALRFPPIRANHSLDVSFYFRTSAPSGVFLENMGGPFCRWRRPYVRVELNTSRDVVFAFDIGNGDENLTVHSDDFEFNDDEWHLVRAEINVKQARLRVDHRPWVLRPMPLQTYIWLVYDQPLYVGSAELKRRPFVGCLRAMRLNGVTLNLEGRANASEGTFPNCTGHCTHPRFPCFHGGRCVERYSYYTCDCDLTAFDGPYCNHDIGGFFETGTWMRYNLQSALRSAAREFSHMLSRPVPGYEPGYVPGYDTPGYVPGYHGPGYRLPEYPRPGRPVPGYRGPVYNVTGEEVSFSFSTNSAPAVLLYVSSFVRDYMAVLIKEDGTLQLRYQLGTSPYVYQLTTRPVTDGQPHSVNITRVYRNLFIQVDYFPLTEQKFSLLVDSQLDSPKALYLGRVMETGVIDPEIQRYNTPGFSGCLSGVRFNNVAPLKTHFRTPRPMTAELAEAMRVQGELSESNCGAMPRLVSEVPPELDPWYLPPDFPYYHDDGWIAILLGFLVAFLLLGLVGMLVLFYLQNHRYKGSYHTNEPKATHDSHPGGKAPLPPSGPAQAPAPTPAPTQLPTPAPAPAPAPASGPGPRDQNLPQILEESRSE

SEQ ID NO: 29 - DNA, 334 bp, synthetic construct
  source 1..334: mol_type = other DNA
aagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcg

SEQ ID NO: 30 - DNA, 69 bp, synthetic construct
  source 1..69: mol_type = other DNA
gagcagaaactcatctcagaagaggatctgcgtacgcggccgctcgattacaaggatgacgacgataag

SEQ ID NO: 31 - protein, 1384 aa, Homo sapiens
MMHLRLFCILLAAVSGAEGWGYYGCDEELVGPLYARSLGASSYYSLLTAPRFARLHGISGWSPRIGDPNPWLQIDLMKKHRIRAVATQGSFNSWDWVTRYMLLYGDRVDSWTPFYQRGHNSTFFGNVNESAVVRHDLHFHFTARYIRIVPLAWNPRGKIGLRLGLYGCPYKADILYFDGDDAISYRFPRGVSRSLWDVFAFSFKTEEKDGLLLHAEGAQGDYVTLELEGAHLLLHMSLGSSPIQPRPGHTTVSAGGVLNDQHWHYVRVDRFGRDVNFTLDGYVQRFILNGDFERLNLDTEMFIGGLVGAARKNLAYRHNFRGCIENVIFNRVNIADLAVRRHSRITFEGKVAFRCLDPVPHPINFGGPHNFVQVPGFPRRGRLAVSFRFRTWDLTGLLLFSRLGDGLGHVELTLSEGQVNVSIAQSGRKKLQFAAGYRLNDGFWHEVNFVAQENHAVISIDDVEGAEVRVSYPLLIRTGTSYFFGGCPKPASRWDCHSNQTAFHGCMELLKVDGQLVNLTLVEGRRLGFYAEVLFDTCGITDRCSPNMCEHDGRCYQSWDDFICYCELTGYKGETCHTPLYKESCEAYRLSGKTSGNFTIDPDGSGPLKPFVVYCDIRENRAWTVVRHDRLWTTRVTGSSMERPFLGAIQYWNASWEEVSALANASQHCEQWIEFSCYNSRLLNTAGGYPYSFWIGRNEEQHFYWGGSQPGIQRCACGLDRSCVDPALYCNCDADQPQWRTDKGLLTFVDHLPVTQVVIGDTNRSTSEAQFFLRPLRCYGDRNSWNTISFHTGAALRFPPIRANHSLDVSFYFRTSAPSGVFLENMGGPYCQWRRPYVRVELNTSRDVVFAFDVGNGDENLTVHSDDFEFNDDEWHLVRAEINVKQARLRVDHRPWVLRPMPLQTYIWMEYDQPLYVGSAELKRRPFVGCLRAMRLNGVTLNLEGRANASEGTSPNCTGHCAHPRLPCFHGGRCVERYSYYTCDCDLTAFDGPYCNHDIGGFFEPGTWMRYNLQSALRSAAREFSHMLSRPVPGYEPGYIPGYDTPGYVPGYHGPGYRLPDYPRPGRPVPGYRGPVYNVTGEEVSFSFSTSSAPAVLLYVSSFVRDYMAVLIKDDGTLQLRYQLGTSPYVYQLTTRPVTDGQPHSINITRVYRNLFIQVDYFPLTEQKFSLLVDSQLDSPKALYLGRVMETGVIDPEIQRYNTPGFSGCLSGVRFNNVAPLKTHFRTPRPMTAELAEALRVQGELSESNCGAMPRLVSEVPPELDPWYLPPDFPYYHDEGWVAILLGFLVAFLLLGLVGMLVLFYLQNHRYKGSYHTNEPKAAHEYHPGSKPPLPTSGPAQVPTPTAAPNQAPASAPAPAPTPAPAPGPRDQNLPQILEESRSE

SEQ ID NO: 32 - DNA, 5537 bp, synthetic construct
  source 1..5537: mol_type = other DNA
gaaaccggcgcgtgccaggagacagaggctggggaaggggggaggtgagaggaaagagggtggaaaggagaggatagagagagaagagcggaggaccaggaaccagagagagagagagagaaaagagagaggagagacagagcgcttgggggcgaaaggagagagggagggaagggtgggtaaggaggagagagcggtctgctgcaaaccccaggaggagagcttggagcccaagccagaactcgagccctagccggagccgttcacagggaggcggctgccgggaccgtcagccctgcatgatgcatctccggctcttctgcatcctgctcgccgcggtctcaggagccgagggctggggctactacggctgcgacgaggagctggtgggtcccctgtatgcacgctccctgggcgcctcctcctactacagtctccttactgcgccgagattcgccaggctgcacggcataagcgggtggtcaccacggattggggatccgaatccctggctccagatagacttaatgaagaagcaccggatccgggccgtggccacacagggctcctttaattcttgggactgggtcacacgttacatgctactctacggcgaccgagtggacagctggacaccgttctaccagcgagggcacaactcgaccttctttggtaacgtgaacgagtcggcggtggtgcgccatgacctgcacttccacttcactgcgcgctacatccgcatcgtgcccctggcctggaacccacgcggcaagatcggcctgaggctcggcctctatggctgcccatacaaggccgacatactctatttcgacggcgacgatgccatctcctaccgcttcccgcgaggggtcagccgaagcctgtgggacgtgttcgccttcagcttcaagaccgaggagaaggacggtcttctgctgcacgccgagggcgcccagggcgactacgtgacgctcgagctggagggggcacacctgctgctgcacatgagcctgggcagcagccctatccagccaagaccaggtcacaccaccgtgagcgcaggcggagtcctcaatgaccagcactggcactatgtgcgggtggaccgatttggccgcgatgtaaatttcaccctggacggctatgtgcagcgctttattctcaatggagacttcgagaggctgaacctggacactgagatgttcatcggaggtctggtgggcgccgcgcggaagaacctggcctatcggcataacttccgcggctgcatagaaaacgtaatcttcaaccgcgtcaacatcgcagacctggccgtgcggcgccattcccggatcaccttcgagggtaaggtggcttttcgttgcctggacccggtaccgcaccctatcaacttcggaggccctcacaacttcgttcaagtgcccggtttcccacgccgtggccgcctggcagtctcatttcgcttccgcacctgggacctcaccgggcttctccttttctcccgtctgggggacgggctgggccacgtggagctgacgctcagcgaagggcaggtcaacgtgtccatcgcgcagagcggccgaaagaagcttcagttcgctgctgggtaccgactgaatgacggcttttggcacgaggtgaattttgtggcacaggaaaaccatgcagttatcagcattgatgatgtggaaggggcagaggtcagggtctcatacccgttgctgatccggacagggacctcatatttctttgggggttgtcccaagccagccagtcgatgggactgccactccaaccagacggcattccatggctgcatggagctgctcaaggtggatggtcaactggtcaacctgactctggtggagggccggcggcttggattctatgctgaggtcctctttgatacatgtggcatcactgataggtgcagccctaacatgtgtgagcatgatggacgctgctaccagtcttgggatgacttcatttgctactgcgaactgacgggctacaagggagagacctgccacacacctttgtataaggaatcctgtgaggcttatcggctcagtgggaaaacttctggaaacttcaccattgatcctgatggcagtggccccctgaagccatttgtagtgtactgtgatatccgagagaaccgagcgtggacagttgtgcggcatgacaggctgtggacaactcgagtgacaggttccagcatggagcggccattcctgggggctatccagtactggaatgcatcctgggaggaagtcagtgcccttgccaatgcttcccagcattgtgaacagtggatcgagttctcctgctacaattcccggctgctcaacactgcaggaggctacccctacagcttttggattggccgaaatgaggagcagcacttctactggggaggctcccagcctgggatccagcgctgtgcctgtggtctggaccggagctgtgtggaccctgccttgtactgcaactgtgacgctgaccagccccagtggagaactgacaagggactgctgacctttgtggaccatctgcctgtcactcaggtagtgataggggatacgaaccgctccacttctgaggcccagttcttcctgaggcctctgcgctgctatggcgatcgaaattcctggaacaccatttccttccacaccggggctgcactacgcttccccccaatccgtgccaaccacagcctggatgtctccttctacttcaggacctctgctccctcgggggtcttcctagagaatatggggggcccttactgccagtggcgccgaccttatgtgcgggtggaactcaacacatcccgggatgtggtcttcgcctttgatgtggggaatggggatgagaacctcacagtacactcagacgactttgagttcaatgatgacgagtggcacctggtccgggctgaaatcaacgtgaagcaggcccggctccgagtggatcaccggccctgggttctgcggcctatgccactgcagacctacatctggatggagtatgaccagcccctctatgtgggatctgcagagcttaagagacgcccctttgtgggttgcttgagggccatgcgtctgaacggagtgactctgaacctggagggccgtgccaatgcctctgagggtacctcacccaactgcacaggccactgtgcccaccctcggctcccctgtttccatggaggccgctgcgtggagcgctatagctactacacgtgtgactgtgacctcacggcttttgatgggccatactgcaaccacgatattggtggtttctttgagccgggcacctggatgcgctataacctacagtcagcgctgcgctctgcagccagggagttctcccacatgctgagccggccagtgccaggctatgagcctggctacatcccgggctatgatactccgggctatgtgcctggctaccatggccccgggtaccgcctgcccgactacccccggcctggtcggcctgtgcccggttaccgtgggcctgtctacaacgttacgggagaggaggtctccttcagcttcagcaccagctccgcccctgctgtcctgctctacgtcagttcctttgttcgtgactacatggctgtgctcatcaaggatgatgggacccttcagctgcgatatcagctgggcaccagtccctacgtgtaccagctaaccactcgaccagtgaccgatggccagccccatagcatcaatatcacccgtgtttaccggaacctcttcatccaggtggactacttcccactgacagagcagaagttctcgctgttggtggacagccagttggactcacccaaggccttgtatttagggcgtgtgatggagacaggagtcattgacccggagatccagcgctacaacaccccaggtttctcaggctgcctgtctggtgttcgattcaacaacgtggctcccctcaagacccacttccgaacccctcgacccatgactgctgagctagctgaggcccttcgagttcagggagaactgtccgaatctaattgcggagctatgccacgtcttgtttcagaggtgccacctgagcttgatccctggtatctgcccccagacttcccctactaccatgatgaaggatgggttgccatacttttaggctttttggtggcctttctgctgctggggctggtgggaatgttggtgctcttctatctgcaaaatcatcgctataagggctcctaccataccaatgagcccaaggctgcccacgagtaccatcctggcagcaaacctcccctacccacttcaggccctgcccaggtccccacccctacagcagctcccaaccaagctccagcctcagccccagccccagccccaactccagccccagcccctggcccccgggatcagaacctaccccagatcctggaggagtccaggtctgaatgagtcagaagggcttctgggaccaattccagctcctgacattcccccagtcctgcctctcccccatcctatcagggacatttggctcctcttagctggctctgctcatccagaggatattcccccatcccccccccatcaagtttggtgggcagagctacagatgggacccaagggagtggccgagcctcactgcctaaaccaatgcccttctcatccctgtttccccaggctcctggctgtttatctgccccaaaggagaagcctcatggggttgacataggtcctttctgccatctctgttccagctgctgtcagggattaacaacagagtgtaggggagattaactgcctcccttccaatagacactatcagcagggacagatgtgtgggagtgcagggctgcagagggtatggggggaggaggctgctaaaccctatcccccagcctcccccctgccctgaagatcttccatttgcttccactcagctggaggctcaagagggcttgatggctgtcccctgcccccctccttttgttttgtacacagagaccaagaggcctcagtttagcaccttagtacctccgctgcttcacttgctttagccaaagccataaaaaacctgcaacgtagagaaaataatgcagataccctgactagccagccctctactcctccaaccttttccaagatatgcaatggcctttgtgcctgcccaaaggcttcgccccctccagtgcatgaggaaccctctttcctccgctcagagatgctgcttcatttacccaggaggtcatattctttatatatattttttgttgcaaagtgtctctctagagaaactctatatattattcgaatttttaaattatttgtttatatataaaagaaaagctcaattggctgtgctgtcctgtgctgtgctgtgcccacagtctgtagtttgcctttcctgtgttggaagtgtcgtgagtcctgctggtcacccaccagcctggtccggcctccactctgaagcagggttgagcctgctgtgctctcagacgcggagccgttccaataaagcagagtggcagagcccca

SEQ ID NO: 33 - DNA, 16122 bp, Mus musculus
  source 1..16122: mol_type = genomic DNA
ccttgtaacagagctctggcagcggcccaaccgaataggcctcctcatcgtacccggagtaaagtccccaggagacctggtggcatcaaaatgagaagagggaccgtgtaagacattcccacacccttccatctcacataggtgcctcattcaaagcctcaataccagatgactctctctctctctctctctctctctctctctctctctctctctctccagattcttgctagtgtgccctgaacaggcagttaagcttcgcttgcagtctctctaactctgcttccagcttcctggatgaccttgagcagccaccggctttttctggacgccacttcctaaatgtgttttacccggtgtagaaaagccccgttcagcccacctggcaagctagcagtgagcactgttgaaaccgtgcaagataatagacaacagaaacatctaactcgccctagagcaagcatttgtattattaataaatctcagcgcttctgttcgctttgcagtgccctctcctggaaagtaagtcagatgcagggctttgcttcagccctggaccctagacttcaagcttaggctaaaggggcttctgtttcctttcccttctctcctccccaccctactgttagagcacagatgtattgaaaacattcacaccaaaggggtcttaggtaggatttgtaggctcctcacaggtgctagatgacagaggcctgctgtcaggcagggaaccccatccctttccccttccccaccctctctgcaacccttctccccctccctgccccgactcccacctgctctgtgggcttggtccccatctctggctacaagggtttctgaggtacagccacagaggctaaagttaaataactagcgggggcaggaaggaaggggtggggaggagggccaaggaggagggttcacgtttccatcccaccttgcccgatttctatctagtcaaaggaaaaggaatgcttgcatggtcttgcacaagttttgttttcctctgagccttcgctgtgtaaagtacagtaagatgaaagcacggagttgacagttacaaaggtgtccagcactgagccaggagcacgattcaccttcagaaaacatttttatttttatttattttatttattttttttttagattttaaatttgcccttcattccatctctaggtgtccttcttcaaagctttttccttttcatgaaaatgctaggctcacctcgcctcctctgacttctcttttttaccaccctgtagccccagtccttgtcagccttgggttaattgggggtatttcccaacggcaggttgtctaccccgccctttcgtaatagggctgaggcatgcgcttgcgcggggtccaggaaaaaacgtgcgtgccaggagaaaggctgggggaagggggcagagagagggagggagagggagggagagaaggagagagagagagagagagagagagagagagagagagagagagagagagagagagagagagagattgggggtaggagagagggaagggtggataaggacggaaaaaagctttggtcagccgtgaacccaggagaaaagctgggggcctgagccagaacgggagccctagcggcgcaacaaggctgacacccagcgttggtcagctccgcatgatgagtctccggctcttcagcatcctgctcgccacggtggtctctggagcttggggctggggctactgtgagtgctggtctcagaggcaggtgggatttggtgcaggaatctggagcttgtagggcgagtcctacaccgcattgcggctgggtggtcgccggtccttccctgtacgcgccagctcgctcccgtctttttcctacctctccccagcgctccgcaatgcagctttgaggctgctgagcgcgcaccactgctgtctggtgctgatctctgatctgaggaaccagtcagcccaggggatttggagctgttaatccaagcgggagccggtgcctaggtttgaggttaaggttggctctaagtgtggtactggtgacccaggctagagttgaggcttttccctaaggggctggggctagaattgggagctgacccaggaactaggtagggtttgtggttgaggctgggttttatggctcgaattcaaataggagttattgaccctgactaaggccaaggatgacgctaagagtgggattgggggtgggaagaagcggggaggggtagattgggagtaaagtctgatttggggattggtgcatgtttcagtgacaggggtgctgatgatcggaagtgatggtaaagctggggttaatttcatgttctgagacccatttaggttttaaatacgctgaagtttggggactatatttaagggggtactgagccagggcaggcactgggctagcaagaatctggctttacacctatagatggaggcatagcgttaccctaagaggggaaggtaagctcccgcggaagcccaggttgagtgcacagctttgaggtagggctgagtcagccacaggggggcagtgaagaggtgctttaactactaagtgggcacagcctttggaagaggggggtcacactaacaagctcatttcctcgccccaccccccactccagacggttgcaatgaggagctggtggggcctctgtatgcacggtctctgggcgcttcttcctactatggactctttaccacagcccgctttgcccggctacatggtgagcttacccaaccaaggaggggaggaggactccagagagctgagagagggtagtgggtggtaaaagcccttgccccttctgtaggcatcagtggatggtcgccccggattggggacccgaatccctggctgcagatcgacttaatgaagaagcatcggatccgggctgtggccacacagggagcttttaattcttgggattgggtcacacgttacatgctgctctacggggaccgtgtggacagctggacaccattctaccaaaaagggcacaatgcggtactctcagtgcccaggcacacatacttggggagccttccccagctctgggccctgggctgggcactagcctgaagggaaggggaggggaggggactctactcttggaatttagtcagctcggactgtttttccaccaatcataaggattaacgtactttagggtaaaacctaggagagagcttcctgagttcagtgacattagactttcactttgaagagataattggatagtctagaccaggaaaactagtcctgtgtgtaatcatggagacatggggatggagtaaacgcctgtttctgccccagtcaaggttgggggcattgctgccagagaagctaagggtggggacatggttactactgttccaaatccccacagaccttcttcgggaatgtcaatgactcggcagtggtccgccatgaccttcactaccactttacggctcgctacatccgcatcgtgccactggcctggaacccacgcggcaagattggcctgaggctgggcatctatggttgtccctacagtaaggctgtggaggccagggggtggaggtgaggatcctgagagggaccatcttcctggtcccctgcccacccacggtcctttacacagcatccagcatcctgtattttgacggcgacgatgccatctcataccgcttccagcgaggcgccagccaaagtctttgggacgtgttcgcttttagtttcaagacagaggagaaggatgggctgttgctgcacaccgagggctcccagggggattatgtgacgcttgaactgcaaggggcgcacctgctgctgcacatgagcctgggtgagcttcagaaccccacagggggtgccctgactcaggttgctctttcccattaactgtctgaaccgaaactatcctttcgtagcgcttggggctagcaaagcagggggcgtggagtcaggagaactggcttcggtttagcaaccatgtggtaaccttgaacatggcatcataaacacatgcagacacacaaaatgcttttttctatctcatttgtctttcataacactcgtgtaatgagatgcaagcagcggcctcctctccgctccaggctttgactgggtcctgggtagcctctactctgtgttgagagttgctgctggcatgggagctagggcctgctagagtcctcaccctgtcccgcattgccctcaggcagcagtcccatccagccaagacctggtcacaccacggtgagccttggtggcgttcttaacgacctaagctggcactatgtgcgggtggatcgatatggccgagatgcaaatttcaccctggatggttacgcccatcactttgtgctcaacggcgactttgaaaggctgaatcttgaaaatgaggtgaccaagggcggggcaccattttggggatgtgacaaaggataaatgtgtgtggggagggatccactccctatcctaccacacgtgccagagagtcagttcttattgtcattgttctggtcctcactggagatctcctaagatgggtctagaaattcagatttggggtacgggggggggggagtagaaaattgttccctagtctcctcccacaattctgtacggttgataaagaccatgtctccatcttcattttaagtgtctaagacagacaatttgacctgctgaaggcctacagccaactagtgctgatgtttaaggcttaaacctgaacgcaagctttattagcactcgtcctggggaaatgtgttccaagactttctgaagccaggcctagtgcctgtcggaggaaagggtacttactgcgcatgcgcacagcaaaggcaggtgttcccctaagcccatggaaatgtctcttcatctgcttcctacagatattcatcgggggtctagtgggcgcagcccgtaagaacctggcctaccgccataacttccgtggctgcatagaaaacgtgatctacaaccggatcaacattgcagaaatggcagtgatgcgccattcgcggatcacctttgaggccagtgggcagggagatctgggaggacagggacactaaagccacttggaaagccaagaagcagagaggaaagaggtcttgttgggaatgagactcactctgaaggccagattctcagatggggggggggggggagtaccacgaactcagagacctaagtaccgttcaccccgttgttaagctgtgaatgggtccagttcatagacctcgaggaaaatgaggtgcagaggctgagaacagtttactaggagcatgtgactccgaagtgacagggtagtcactgaactgtctgtcactcgaagtcttttgataagctttcctctttggtcgactcagaaacttgtgtgtatgctccacagcctttgggctaaattaggcttgtgctatatgatactgctcttagcactctgagagggatttttttttttttttttttttttttttttttttttttttgcttttgcttttgctttatggacattgggattagcaacaaaaagcaaacacggggtgggggggtcggaggtctgaaagcaggcttgtttgagtctcttccacaaacatgggcatatttgcacagggaatggaaggaccacagttggaattcttagggagtgagaatattgatggcgagtcctgatgcatccatccattctttttttttttttttagggtaatgtggctttccgttgcttggatcccgttccacaccccatcaacttcggaggccctcacaacttcgtccaagtgcctggctttccacgccgaggacgcttagccgtctcttttcgtttccgcacctgggacctcacagggctgctccttttctcccacttgggggacgggctgggtcatgtggagctgatgcttagcgaagggcaagtgaatgtatccatcgcgcagactggccgcaagaagcttcagtttgctgctggtgaggggcccaaggggaggcagaagcaggctggaaaagggggggggatctggaaggaacaggtgtgttccgcttctgggggtcctgaagatgcttgccagttataaccagctttgcacactatatcaatgtctagggctgcggatagtgaagggagccgacagaggtgccactacgaagggagtgaagtgtaataatggggtatattagactgctgttgggattacagagtgaggctgctgcttccccactcgagtccatcttttctctggtggcttgtgataatttacgcccataatcccaacatctgagaggacaggacaggaaaaccacaagtttgaggccagtcaggactacatagtgagttctaggccaggctggaatacagtgagaccttgtctcaaaagcacagcaaggggctgtgctgtgcaagaacagtaccattagctactaaactgctctccacgccccctgtgattccatgaggatccagcgcctccttctggcttcccagggtactgcactcatgcacacagttccaaaaaaaaaatttttttttttttaaaaagacgggtggggatgggggggaaaaacaaaccaatcttctcatattctggttactggctctacccacttagcccatcctcctttctcattcagggtaccgcctgaatgatggcttctggcacgaggtgaactttgtggcacaggaaaaccatgcagtcatcagtattgatgatgtggaaggggcagaggtcagggtttcatacccactgctgatccgcacagggacttcatacttctttggtggtaagtgaggagcaactttgtcctgtctctgggtgggaaggtcctatttcacttctgatggggccacatagagagtattggagagaccaatcctcctttcacctctgggaccttgggctgtaggtccttgccattatttgcactttgattcttgcatctttttacttatgtatggctctttcttggcatttccccagtgggggcggggcatctgggagctctcctgggaaggaccttacaggtgtggttgctccaggttgtcccaaaccagccagtcgatggggctgccactccaaccagacagcattccatggctgcatggagctgctcaaggtggacggtcaactggtcaacctcactctggtagagtttcggaagctcggttattttgctgaggtcctctttgacacatgtggcatcacagacaggtatctagaagtgacccctagttgcctagcaagtgacctcacttccatttttctttttaaagtaatctcccttctcctgatgtccatgcccccacctacacacacacacacacacacagtcttccttcacctctttcctatttctttctgtaaagttgcaggtacccaggattgaaagaaaatttctattcagtcctaaggtttttgtttggcagggtctgtgtactttcctttcttttggcacgaagaggaccgcacttgatccctgtagactacctagtgtccgtgtctggggttgtacgtagtcagttaggtgggaagtgagccggggtcctggtcctttgggaagctagtcccaagcttccttcccttggtgcttgaccccttccacagatgcagccctaacatgtgtgagcatgacggacgatgctaccagtcttgggatgacttcatctgctactgcgaacttaccggctacaagggagttacctgccacgaacgtaagccaaatggtatagggagtgaggggaaggttgttggggatcaggaagctttctagaggtggtagggatggccattcttctagccttctagcttcacaattgtcttgccgcccccactagcattgtacaaggagtcctgtgaggcctatcggctcagtgggaaatattctggaaactacaccattgatcctgatggcagtggacccctgaagccgtttgtggtgtattgtgacatccgaggtaagtgacttttgatgggcgagggagagggttgactaggcaacatatgagatgtgggggtcagagaagctgtaggaggggtcaagagcaaaccatagtggggggtaaaggatggtgtggaaaagggacatctaaaaatgtgtagtgtagccttacagctaacttcccatttctggtcattccagcagcctcatctccatcctctggtgtggagggcccaaggctggactgaagggaagtgcttagccacttttataggctctagctaggttggtaaggagggagacagaatgttcatgtccagccaaagctctgtcccttctctttcagagaaccgagcgtggacagttgtgcggcatgacaggctgtggaccactcgagtgactggttccagcatggaccggccctttctgggggccatccaatactggaatgcctcctgggaggaagtcagcgctctggccaatgcttcccaacactgtgagcagtggatcgagttttcctgctacaattcccggctgctcaacactgcaggttgatgctggtgacagggagaagagtcttgggtgggggtaaagtcagggctgcttgaacccaggtatgaggaactcagaggctgatgaagaggcagggactggaagctgcttggtgtcttccccaggaggctacccctacagcttttggattggccgcaatgaggaacagcatttctactggggaggctcccagcctgggatccagcgctgtgcctgtgggctggaccagagctgtgtggaccctgcactgcactgcaattgtgatgccgaccagccacagtggtgaggacaggagggccggcttttggagacttgcaggggcagctgagaggcacggtagagtcaccgaatcctatattcccacaggagaacagacaaggggctcctgacctttgtagaccatctgcctgtcactcaggtagtggtaggtgatacaaaccgctcaaattctgaagctcagttcttcctgaggcctctgcgctgctatggtgaccgtgagtggcagactcctttggtgtgcctgtccagtactgtttccctgctccactgggaatctctaagccacgtggccagatgctcctgctgtgacctgttccctgctttccttgtacctctgcaccttattcacccacatcttaatctctcttcaaagagtctcccaagctctccctgctctaccctcccttcctcctgcccactgaggctccttgctcctccaccccccagctcccttgacaaggccttcctcaccctgtttttgcaggcaattcctggaacaccatctccttccacactggagctgcactgcgtttccctccgatccgagccaaccacagcctcgatgtctcattctacttcaggacctcggctccctcgggtgtcttcctagagaacatggggggtcctttctgccggtggcgccgaccttacgtgagagtggagctcaacagtaagtgggcagggctaggaggaatgtgggtagagatggctgagggggctacgttagggagcagggcgctcacccttctgagaggtggcagggagaagattggagatcactctggtcctagacttattgtcttggtgtctggctgtgtggccagagacccttggacacatgctccgcccctgtgggccctttacccctttacttgtgaaaaaggagctaggttcttgaggtctcaaccatctctaattagctgtgcttctagcctgggcatttaaactggcaaattgattgcaaggaatctgtaacacaggggaacagattgacatattgtggttttaagccttttgtagattttgtcttaggctgtgaacaaagtggagggccatagggggacccccatcaggtgaggagattgtattgggcaagaatactgtgagggatctgggggatccgagtccccctttacccccttaactactgtcatgctgtgcagcatcccgggatgtggtctttgcctttgatattggcaatggggatgagaacctgacagtgcactcggatgactttgagtttaacgatgatgagtggcatttggtccgagctgaaatcaacgtgaagcaggcccggctgcgagtggatcaccggccctgggtgctaaggcccatgcccctgcagacctacatctggctggtgtatgaccaacccctctatgtgggtaagcaggaacccaggaggagggagaaagcctccatgacattcccacactctcatgggccatttttccaggaagcccatgttctaatcctctatgcccatgtgagccaaggggccatgtatctccttcgctccctcatatcctagtccatcctcctagcccctcctgggctcccttctcgctcacaatactgagtcctgtctctggctctatagtcattttaggggagctcttgggatgttctacctttgacctacacccccagcctcagcctctctacttactcctgaccaggatctgcagagcttaagaggcgcccttttgtgggctgcttgagggccatgcgtttgaatggagtgactctgaacttggagggccgtgccaatgcctctgagggcaccttccccaactgcaccggccactgcacccacccccggttcccctgtttccacggaggacgctgtgtggagcgatacagctactacacatgtgactgtgacctcacagcttttgatgggccatactgcaatcacggtcagtgctgctagttactagggacgggacgggcgtggggaggtgggggtgacaaggagccttagggcataatgggagtttagaggggagattacaagtcagcagtcacggaaaactagaattgtcagtagacttctgagttcgtaaacggggcaatcaggccaccagaatatgctgggtaactccaaagtcataatgaaaaacagagggacagtagagctagctgacacaagagccccaacattagtcaaataactccctttttcccactgttgtcaaggtcacacagactatatgctcccagaccccatgcacatctactttgtgtacacgcagatgtatgtgcatgtatgtatggaggtgcatgcacgtgtgtacgaaggtcaggggtcaacctcaggtgttgtttctcaggtgcatccaccttgtgttttcagacaggtctctcactgggacctggaactcagtaattatgtaggctagctggacagtgagccacagagatcctcttgccttggcctccccagcgtgggattccaactgtgtgccatcacaactaacctgtgtgtgggtgctaaggattgaattcgtggcctcacactggtacagcaagcactttatcagctgaacaagtgtcctagctctggtagtgactttggcttcacaatacatttctcccctggccatttcctgtctgactatcactgaattcctaaaacacatctacaagctgactctgggttggggatgaagcttgaaattcaacatatccagactctattttcttaaggctggcccaagtaggttactgtgggtggagtctggtaatttagggagacacaggaagtaggaataacaacacataattggatggagtcagggtatcttaattctcatctcggccatccacatacaccacattcttgggggaagttacgacccatctttagggttcagtgtcctcacctgtaaatgcagggataagaccagacgacctttcaagtttcttataccattattattattacttagtgctagatgagttggtgtgggcctgtaatgcgagcacttttcaggctggctgtaaatttgagctacaaagacagtgtccccaaaattgaagtggggaaaaagagcaaatagtggcagatacagatgtgtgcgtgggtcggtgacataaaaaccctttgaacttgtgactgagaagttatctctgcttcagatattggtggattctttgagaccggcacatggatgcgctataacctccagtcagcactgcgctctgcagcccgggagttctcacacatgctgagccggccagtgccgggctatgagcctggctatgttccaggctacgacactcctggttacgtgcctggataccacggccctgggtaccgactgcccgagtacccgaggcccggccggccagtgcccgggtatagggggcctgtctacaatgttactggagaggaggtctccttcagcttcagcaccaactctgctcctgccgtcctgctctatgtcagctcctttgtacgtgactacatggctgtgctcatcaaggaagacggtaagtgctcctcggctctctccctgcaggtgcccaggctcccacagagattgactaatgcaaagatccagtcctttagacgatgagggactgtggaagccgagacaagagcccatgtccctcctcccaccccacagggaccctacagcttcggtatcagttgggcaccagtccctatgtgtaccagctaaccacccggccagtgactgatggtcagccccatagtgtcaacatcacccgggtctaccgaaacctctttatccaggtacaggtggaagggatgggccagttaagtcatagcctcaccaccctggctatggcaccaagaaaacgtctaatgattacatatctgaaacacagctcagtatacgagggattttcacatgtatcacctctcatgaccctcacatcagctctgtgaagtaggtgtggctagaaattacactgaggccgggcagtggtggtgcatgcctttaatcccagcactcgggaggcagaggcaggcggatttctgagttcgaggccagcctggtctacagagtgagttctaggacagccagggctacacagagaaaccctgtctcgaaaacaaacaaacaaacaaacaagaaattactttgagcttcactttacaaaccagtaaacaggctggtaagcagaaggtaaagtgtcttccgcagtcagtctcgtaggggatgctgacgtagcttagtcgacacagggcttgtctactaggcatgaagccctgggtcccatccccagcactgcacagacatgtcatctcagtatgtgggaggctggggcaggaggaacagaagtttaagatcatccttgactacatggcaagtctgaggccagcctgagctacatgaaaccctgtatgaaaagggggtgctggtgagatactcagtggggagagcctgatgacagagttttatccctcgatcacattggaaaaggggagaaatgacagtcacgttgttacctgaactccagacaaactgtggcactccccataagaaataaagtgtagttgggtacagtagctcaagcctttaatcctaagccttgggaggcagagggagacatggacctctgactctgaggccagcctggtctacactgaattccacgacagccagggctatgtagagagaccctgccctcccccaaccccacccccacccccaccaaatcaaaccagaaatgaaagtcaacctaccagcaaactgcaaagccaagggtcaaaaccagattgagtcaggtgtcaaatcctagctctgggcccaggggtttgttcgtttttgagcaaggctagtttgggcagagtgaatcccttccttggtcttggctgtgtccatccagggttttcctccctccctccctcacttctaccctaggtggactacttcccgctgacagaacagaagttctctctcctggtggacagccagctggactcacccaaggccttgtatctagggcgtgtgatgggtaagctgcttgtgaggatggatctggggagaggagcaagcagagagtggggaagcagaaagagcagcgagggtagtgggaggggaatggaagaggggcaaggcttctctgggtgaaccctgatacctctttcttggccgcctctccctcagagacaggagtcattgacccagagattcagcggtacaacaccccaggtttctcaggctgcctgtctggtgtccggttcaacaatgtggctcctctcaagacccatttccgaacccctcggcccatgactgctgagctggcggaggccatgcgggttcaaggagaactgtcagaatctaactgtggagctatgccacgccttgtctctgaggtgccaccagaacttgatccctggtacctgcctccaggtgaatccctagacacaagtgaagatgggggtggggtggggtaacagtgatgggaaatggctctgaatacagaggcagcggctcttaatcagcctttcctcctctttagatttcccatactaccatgacgacggatggattgccatactcttaggttgtaagtagcaccgaccatgaacccgacttcctaagaccacctcgctagcatcctccctggctcctgtcctcagaagtggggtggggggaggcattagctgtgagatggcaagaaggcggcagggacaggttatgctgacatcagtatttagctgggggaggactaggcagctataaatttttaattgagatgaaatattaaaatgcagccccagttacaggatgaggaaaaatagaaatgtagtaacatctagagaaataagaaattggagaagccatcagagatagggaaagggtaaagtggacagtggataagccaagaactcagacattctagacagagccatgcctgacacagttatgtttgctatacccgggtgaacatgatgctttggccgagcgtgtatgtgtgtaccccgaaggccttccctaagctgttctttttcttccattcagttttggtggccttcctgctgctggggcttgtgggaatgctggtgctgttctatctgcaaaatcatcgatacaagggctcctaccacaccaacgagcccaaggccacccacgattcccatcctggcggcaaagctcccctccctccctcaggccctgcccaggcccctgcccctactccagctcccacacagcttccaaccccagccccagccccagccccagccccagcctctggcccaggccccagagaccagaacctcccccagatcttggaggagtccaggtctgaatgagtcacaagggcttcagggaccaaggccaactcctctaagtcccctcagctcctgccttccccctcccttgccagggacatttggctcttctaagcaggctctgttcaccaggaggattcccctcttgccaagtttggtgcgcagagctacagatgggaccaaagggagtggccgagcctcactgcctaaaccaatgccctgctcatccctctacccccccccccacccgcccccagctccagactgtttgcctgccctacaggagaaagcccatgatgctgagacaggcctttcctgccatctctgtcccagctgctggcaaggattaacaacgaagggcaggggagatgaactgcctcccttcctgtgtacactatcagcagggacagatgtgggggatcgagaggctgctaaaacacaccctggagcctcccctcctcctcctgccctgctgaccggctgtcttccatctgcttcctctcagctggggctgagggaagaacttcaccacccccacccccacctcactcaaccctttttgttcttacagagacaagaggcctgcctcagtttagcactttagtacctccactgcttcacttgcttcagccaaagccataaaaagcctgcctgagtagaaaagataatgcagatgccctgcctagccagccctctactcttccaccccttttcaaaatatgcaatagccttggtgcctgtgcgcaggcctggccctctccaatgcatgaggagccctcccccctctgctcagaaatgctgctgcgtttacccaggaggtcatattctttatatatatttcttgttgcagagtctctctccagagaactatatattactctaattttttaattatctgtttatatataaaagaactcagtgggctgtcctgtgctatgcccagtttgtagcgagctccttctgtgtttgatgtctcatgagtcctgccagccactcacccacgtgctgggcctccactctagtgcagggcagagcctgctgtgccctcaactgagcaggttcaataaagcagcgtggcagagcccc

(19) United States
(12) Patent Application Publication
(10) Pub. No.: US 2025/0248376 A1
(43) Pub. Date: Aug. 7, 2025

(54) METHODS AND COMPOSITIONS FOR TREATING NEUROPATHIES CAUSED BY A CNTNAP1 MUTATION

(71) Applicant: BOARD OF REGENTS, THE UNIVERSITY OF TEXAS SYSTEM, Austin, TX (US)

(72) Inventors: Manzoor Bhat, San Antonio, TX (US); Qian Shi, San Antonio, TX (US)

(21) Appl. No.: 18/856,269

(22) PCT Filed: Apr. 14, 2023
(86) PCT No.: PCT/US2023/065827

§ 371 Date: Oct. 11, 2024

Related U.S. Application Data
(60) Provisional application No. 63/331,557, filed on Apr. 15, 2022.

(51) Int. Cl. A01K 67/0275; A61K 38/00; A61K 48/00; C07K 14/705; C12N 15/86

(52) U.S. Cl. CPC A01K 67/0275; A61K 48/005; C07K 14/705; C12N 15/86; A01K 2207/15; A01K 2217/075; A01K 2217/203; A01K 2227/105; A01K 2267/0318; A61K 38/00; C12N 2740/15043

(57) ABSTRACT

Disclosed herein are methods and compositions useful in treating a neuropathy caused by a CNTNAP1 mutation. Transgenic animal models and cell lines are disclosed for the study of neuropathies caused by a CNTNAP1 mutation. Methods of screening and identifying active agents for the treatment of neuropathies caused by a CNTNAP1 mutation are also provided.

### CROSS REFERENCE TO RELATED APPLICATIONS

This application claims the benefit of the filing date of U.S. Provisional Application No. 63/331,557, filed on Apr. 15, 2022. The content of this earlier filed application is hereby incorporated by reference in its entirety.

### STATEMENT REGARDING FEDERALLY FUNDED RESEARCH

This invention was made with government support under grant number GM063074 awarded by the National Institutes of Health. The government has certain rights in the invention.

### INCORPORATION OF THE SEQUENCE LISTING

The present application contains a sequence listing that is submitted concurrent with the filing of this application, containing the file name “21105_0088P1_SL” which is 77,824 bytes in size, created on Apr. 14, 2023, and is herein incorporated by reference in its entirety pursuant to 37 C.F.R. § 1.52(e)(5).

### BACKGROUND

CNTNAP1 encodes the transmembrane Contactin associated protein 1 also known as Caspr. Caspr is located in the paranode region of myelinated axons, where it flanks either side of the node of Ranvier. Caspr is required for axonal domain organization and participates in the propagation of action potentials and signal transductions along nerve fibers. This organization allows for saltatory action potentials to occur, which increases the speed and effectiveness at which neurons can communicate with one another. Therefore, proper domain organization of the nodes, paranodes and juxtaparanodes is important for neurons to relay information efficiently. Mutations in human CNTNAP1 have been associated with dysregulation and disorganization of these domains resulting in various forms of congenital hypomyelinating neuropathies. Although these mutations have not been fully characterized, it is known that they cause grave consequences to the children with mutations, including slowed nerve conduction, intellectual disability, muscle atrophy, respiratory issues and a high rate of infant mortality. Currently, no treatments exist for the neuropathies caused by these CNTNAP1 mutations, thus, therapeutics are needed.

### BRIEF DESCRIPTION OF THE DRAWINGS

FIGS. 1A-C show the location of the Caspr protein and phenotype of Caspr mutations in children. FIG. 1A shows the location of Caspr protein in myelinated axon. FIG. 1B is a schematic of axonal domains. FIG. 1C shows the phenotype of children who carry mutations in Caspr protein.

FIGS. 2A-B show the generation of Cntnap1 R765C/null transgenic mice using CRISPR/Cas9. FIG. 2A shows a working model for CRISPR/Cas9. FIG. 2B shows the sequences of Arg765Cys mutation in transgenic mice. ACCGCTC is the wild type sequence and ACTGCTC is the mutated sequence.

FIGS. 3A-D show that Cntnap1 mutation leads to loss of nerve conduction and paranodal junction disorganization. FIG. 3A shows reduction in nerve conduction velocity and amplitude in Arg765Cys mutation mice. FIG. 3B shows the loss of fine motor coordination and balance in Arg765Cys mutation mice. FIG. 3C shows the reduction in Caspr protein levels in spinal cord of Arg765Cys mutation mice, with no difference in NF-155 and Contactin protein levels. FIG. 3D shows domain disorganization, reduction of Caspr protein and enlarged nodes mice with the Arg765Cys mutation.

FIGS. 4A-G show that overexpression of wild type Cntnap1 in neurons rescue the phenotypes of Arg765Cys mutant mice. FIG. 4A shows the generation of the LSL-Cntnap1 mice. FIG. 4B shows the confirmation of Cntnap1-Flag in LSL-Cntanp1+ Cre mice. FIGS. 4C-D show that nerve conduction velocity and amplitude improves over time in Arg765Cys rescue mice. FIG. 4E shows that Caspr protein levels in the spinal cord are increased to WT levels in Arg765Cys rescue mice. FIG. 4F shows that domain organization is restored in nodes, paranodes and juxtaparanodes in spinal cord and sciatic nerve in Arg765Cys Rescue mice. FIG. 4G shows that over time, motor coordination and balance improves with an increased length of beam walk in Arg765Cys rescue mice.

FIG. 5 shows an example of a lentivirus containing Caspr.

FIG. 6 shows CNTNAP1 is an important constituent of axonal paranodal junctions in myelinated axons. The juxtaparanodal region is high in potassium channels (K+, red), the paranodal region contains CNTNAP1 (blue), and the nodal region is rich in sodium channels (Na+, green) while lacking a myelin sheath (white).

FIG. 7 shows three Cntnap1 gene mutations were created in mouse lines based on known human CNTNAP1 mutations (C232R, R388P, R764C).

FIG. 8 shows the disruption of the paranodal structure in mutant mice. CNTNAP1 protein (Caspr, red) is disrupted from paranodal junctions in mutant mice lines. The paranodal junction proteins Contactin and Neufofascin155 were also mis-localized in mutant mice (green in left and right panels, respectively). Natch (blue): sodium channels indicating nodal region.

FIG. 9 shows a Lox-Stop-Lox (LSL) CRE rescue mouse line developed to express an inducible wild-type copy of the CNTNAP1 protein. Left: Flag CNTNAP1 tag (green), CNTNAP1 (red), βIV-spectrin (sodium channel regulator marker, blue), overlay. Middle: LSL design. Right: CNTNAP1 protein is detected after rescue.

FIGS. TOA-S show generation of Cntnap1 mouse mutants carrying human CNTNAP1 mutations and their phenotypic analysis. FIG. TA shows a schematic of the Cntnap1 protein and the approximate locations of amino acid changes. Functional domains based on the primary amino acid sequence and homology are highlighted. FIG. B shows the sequence alignment of two segments of the Cntnap1 protein showing high conservation of Cys324 and Arg765 cross multiple species. SEQ ID NO: 3 is LAYRHNFRGCIENVIF; SEQ ID NO: 4 is LAYRHNFRGCIENVIY; SEQ ID NO: 5 is LPGKVNFRGCMENVFI; SEQ ID NO: 6 is LSHKQNFRGCLENILF; SEQ ID NO: 7 is TQVVIGDTNRSTSEAQFFL; SEQ ID NO: 8 is TQVVVGDTNRSNSEAQFFL; SEQ ID NO: 9 is TQVVIGDTNRSSSEAQFFL; SEQ ID NO: 10 is TQVVVGDTNRSSSEAQFFL; SEQ ID NO: 11 is RRLVIGDTNRTGSEAQYSV; and SEQ ID NO: 12 is TRVVVGDTNRTGSEAWFSV. FIG. 10C shows the genomic DNA sequences from wild-type controls [Cntnap1^{C324} (TGC), and Cntnap1^{R765} (CGC) and Cntnap1 mutants Cntnap1^{R324} (CGC) and Cntnapc⁷⁶⁵ (TGC). FIG. 10D and FIG. 10E show the physical appearance (FIG. 10D) and body weights (FIG. 10E) of control C324R/−, R765C/−, KO mice at postnatal day 40. FIG. 10F shows the representative immunoblots showing the protein expression of Cntnap1 in the CNS (spinal cords) or the PNS (sciatic nerves) from Cntnap1^{+/-}, C324R/−, R765C/− and KO mice. P-actin was used here as the loading control.

FIG. 10G shows the quantification of relative protein band intensities for Cntnap1 in the CNS or PNS of +/−, C324R/−, R765C/− mice. FIG. 10H to FIG. 10K show the immunostaining of sciatic nerves from +/− (FIG. 10H), C324R/− (FIG. 10I), R765C/− (FIG. 10J) and KO (FIG. 10K) mice using antibodies against Kᵥ1.2 (juxtaparanodes, green), Cntnap1 (paranodes, red) and PIV Spectrin (nodes, blue). Scale Bar: 5 mm. FIGS. 10N-10Q show the immunostaining of spinal cord from +/− (FIG. 10N), C324R/− (FIG. 10O), R765C/−(FIG. 10P) and KO (FIG. 10Q) mice using antibodies against Kᵥ1.2 (juxtaparanodes, green), Cntnap1 (paranodes, red) and PIV Spectrin (nodes, blue). Scale Bar: 5 mm. FIG. 10L and FIG. 10M show the measurement of relative fluorescence intensity of Cntnap1 at the paranodes in the sciatic nerves and the distribution of relative fluorescence levels of Cntnap1 across paranodes in +/−, C324R/−, R765C/− mice. FIG. 10R and FIG. 10S show the measurement of relative fluorescence intensity of Cntnap1 at the paranodes in the spinal cords and the distribution of relative fluorescence levels of Cntnap1 across paranodes in +/−, C324R/− and R765C/− mice.

FIGS. 11A-Z show that important paranodal proteins fail to localize properly in CntnapC^{324R/-} and Cntnap1^{R765C/-} mouse mutants. FIGS. 11A-11F show immunostaining of sciatic nerves from +/−, C324R/− and R765C/− mice using juxtaparanodal Kᵥ1.2 (green), paranodal NF155 (FIGS. 11A-C) or Contactin (FIGS. 11D-F) (red) and nodal Naᵥ channels (blue). Scale Bar: 5 mm. FIGS. 11G-L show the quantification of the relative fluorescence intensity of nodal Naᵥ (FIG. 11G), paranodal NF155 (FIG. 11I) and Contactin (K) from +/−, C324R/− and R765C/− mice. The distribution of relative fluorescence intensities of the respective proteins across paranodes (FIGS. 11H, 11J, and 11L) from +/−, C324R/− and R765C/− mice. FIGS. 11M-R show immunostaining of spinal cords from +/−, C324R/− and R765C/− mice using juxtaparanodal Kᵥ1.2 (green), paranodal NF155 (FIGS. 11M-O) or Contactin (FIGS. 11P-R) (red) and nodal Naᵥ channels (blue). Scale Bar: 5 mm. FIGS. 11S-X show quantification of the relative fluorescence intensity of nodal Naᵥ (FIG. 11S), paranodal NF155 (FIG. 11U) and Contactin (FIG. 11W) from +/−, C324R/− and R765C/− mice. The distribution of relative fluorescence intensities of the respective proteins across paranodes (FIGS. 11T, 11V, and 11X) from +/−, C324R/− and R765C/− mice. FIG. 11Y shows representative immunoblots showing protein expression of total Neurofascin (NF186 and NF155) (using NF-CT antibody), Contactin, 4.1B in CNS (spinal cords) from +/−, C324R/−, R765C/− mice. FIG. 11Z shows quantification of relative band intensities of the proteins immunoblotted in FIG. 11Y.

FIGS. 12A-L show that Cntnap1^{C324R/-} and Cntnap1^{R765C/-} mutants display severe motor disability and decline in nerve conduction properties. FIG. 12A and FIG. 12B show beam walking motor coordination performance of +/− (control), C324R/−, R765C/− and KO mice. Walking distances traveled by each mouse (the full length of the beam is 50 cm). FIG. 12C and FIG. 12D show motor learning ability of +/− (control), C324R/−, R765C/− and KO mice as measured by Rotarod test (FIG. 12C). The total time spent on rotarod for each trial is shown in FIG. 12D. FIGS. 12E-L show representative electrical impulse traces from P20 sciatic nerves (E) and ankle (FIG. 12I) from +/− (control), C324R/−, R765C/− and KO mice. Measurement and quantification of amplitude (FIG. 12F and FIG. 12J), nerve conduction velocity (NCV) (FIG. 12G and FIG. 12K) and latency (FIG. 12H and FIG. 12L).

FIGS. 13A-P show that Cntnap1^{c324R/-} and Cntnap1^{R765C/-} mutants display hypomyelination and loss of the paranodal axo-glial junctions. FIGS. 13A-D show transmission electron microscopy (TEM) of cross sections from P21 spinal cords of Cntnap1^{+/-} (control) (FIG. 13 A), C324R/− (FIG. 13B), R765C/−(FIG. 13C) and KO (FIG. 13D) mice. FIGS. 13E-H show transmission electron microscopy (TEM) of cross sections from P21 sciatic nerves of Cntnap1^{+/-} (control) (FIG. 13E), C324R/− (FIG. 13F), R765C/− (FIG. 13C) and KO (FIG. 13D) nice. FIG. 13I and FIG. 13J show morphometric analysis measuring the g-ratios of myelinated axons (FIG. 13I, spinal cords and FIG. 13J, sciatic nerves). FIGS. 13K-M show TEM images of longitudinal sections of P21 spinal cords at the level of the paranodal region showing axo-glial junctions in Cntnap1^{+/-} (control) (FIG. 13K), C324R/− (FIG. 13L), R765C/−(FIG. 13M)mice. FIGS. 13N-P show TEM images of longitudinal sections of P21 sciatic nerves at the level of the paranodal region showing axo-glial junctions in Cntnap1^{+/-} (control) (FIG. 13N), C324R/− (FIG. 13O), R765C/−(FIG. 13P) mice. White arrowheads in FIG. 13K and FIG. 13N indicate distinct ladder-like septic junction between myelin loops. White arrows in FIG. 13L, FIG. 13M, FIG. 13O, and FIG. 13P indicate lack of axo-glial septate junctions in CNS and PNS myelinated axons. Scale bars: (A-H)=2 m; (K-P)=0.2 μm.

FIGS. 14A-W show neuronal expression of the wild-type Cntnap1 gene progressively restores the paranodes and axonal domain organization in Cntnap1^{C324R/-} and Cntnap1^{R765C/-} mutant myelinated axons. FIG. 14A shows a schematic representation of the generation of LoxP-Stop-LoxP (LSL) Cntnap1^{Flag} transgenic mice. FIG. 14B shows representative immunoblots showing the expression of Flag-tagged Cntnap1 (Cntnap1^{Flag}) in the spinal cord tissue from control (LSL-Cntnap1) or LSL-Cntnap1^{Flag};Actin-Cre mice. β-actin was used as a loading control. FIG. 14C shows immunostaining of sciatic nerves using antibodies against Flag (a, d, green) and Cntnap1 (b, e, red) and merged image showing paranodal localization of the transgenic Cntnap1^{Flag} with the endogenous Cntnap1 protein (c, f, yellow). Scale Bar: 10 mm. FIG. 14D and FIG. 14E show representative immunoblots showing the expression of Cntnap1^{Flag} in the spinal cord tissues from Cntnap1^{-/-} (control) or LSL-Cntnap1;Slick-H-Cre; C324R/− (TgEx;C324R/−) mice at 2 weeks or 7 weeks after tamoxifen injection. Spinal cord protein samples from C324R/− and KO mice are also included. β-actin was used here as loading control. The relative protein intensities of Cntnap1 from all these genotypes is quantified in FIG. 14E. FIGS. 14F-K show immunostaining of sciatic nerves (FIGS. 14F-H) or spinal cords (FIGS. 14I-K) from C324R/− (FIG. 14F and FIG. 14I), and LSL-Cntnap1;Sick-H-Cre;C324R/− (TgEx;C324R/−) mice at 2 weeks (FIG. 14G and FIG. 14J) or weeks (FIG. 14H and FIG. 14K) after tamoxifen injection using antibodies against Kᵥ1.2 (juxtaparanodes, green), Cntnap1 or Flag (paranodes, red) and Naᵥ channels (nodes, blue). Scale Bar: 5 mm. FIG. 14L and FIG. 14M show the relative fluorescence quantification of Cntnap1 in both the CNS and PNS is shown (FIG. 14L. The overlap of Kᵥ1.2 with the paranodal region is quantified in the PNS (FIG. 14M). (100% overlap means no paranodal junction or separation is observed). FIG. 14N and FIG. 14O show representative immunoblots showing the expression of Cntnap1^{Flag} in the spinal cord tissues from Cntnap1^{+/-} (control) or LSL-Cntnap1;Slick-H-Cre;R765C/− (TgEx;R765C/−) mice at 2 weeks or 7 weeks after tamoxifen injection. Spinal cord protein samples from R765C/− and KO mice are also included. β-actin was used here as loading control. The relative protein intensities of Cntnap1 from all these genotypes is quantified in FIG. 14E. FIGS. 14P-U show immunostaining of sciatic nerves (FIGS. 14P-R) or spinal cords (FIGS. 14S-U) from R765C/− (FIG. 14P and FIG. 14S), and TgEx;R765C/−) mice at 2 weeks (FIG. 14Q and FIG. 14T) or 7 weeks (FIG. 14R and FIG. 14U) after tamoxifen injection using antibodies against Kᵥ1.2 (juxtaparanodes, green), Cntnap1 or Flag (paranodes, red) and Naᵥ channels (nodes, blue). Scale Bar: 5 mm. FIG. 14V and FIG. 14W show the relative fluorescence quantification of Cntnap1 in both the CNS and PNS is shown FIG. 14V. The overlap of Kᵥ1.2 with the paranodal region is quantified in the PNS (FIG. 14W. (100% overlap means no paranodal junction or separation is observed).

FIGS. 15A-H show neuronal overexpression of wild-type Cntnap1 restoration of body weight and motor functions. FIG. 15A show the body weight of age-matched +/− (control), C324R/−, and TgEx;C324R/− at various stages after tamoxifen injection. FIG. 15B show the results of the rotarod test showing motor performance by +/− (control), C324R/−, and TgEx;C324R/− at various stages after tamoxifen injection. FIG. 15C and FIG. 15D show motor coordination performance in beam walking tests measured as walking distances (FIG. 15C) and walking speed (FIG. 15D) by +/− (control), C324R/−, and TgEx;C324R/− at various stages after tamoxifen injection. FIG. 15E show the body weight of age-matched +/−, R765C/−, and TgEx;R765C/−) at various stages after tamoxifen injection. FIG. 15F show the results of the rotarod test showing motor performance by +/−, R765C/−, and TgEx;R765C/− mutants at various stages after tamoxifen injection. FIG. 15G and FIG. 15H show motor coordination performance in beam walking tests measured as walking distances (FIG. 15G) and walking speed (FIG. 15H) by +/−, R765C/−, and TgEx;R765C/− mutants at various stages after tamoxifen injection.

FIGS. 16A-P show that neuronal expression of the wild-type Cntnap1 restores myelination and paranodal axo-glial junctions in C324R/− and R765C/− mutants. FIGS. 16A-C show 7 weeks after tamoxifen injection, TEM of cross sections from the spinal cords of 2 months old +/−(control) (FIG. 17A), TgEx;C324R/− (FIG. 17B), and TgEx;R765C/− (FIG. 17C) mice. FIG. 17D show morphometric analysis showing g-ratios of spinal cord myelinated axons +/−(control), C324R/−, TgEx;C324R/−, R765C/−, and TgEx;R765C/− mice.

FIGS. 17E-G) 7 weeks after tamoxifen injection, TEM of cross sections from the sciatic nerves of 2 months old +/−(control) (FIG. 17E), TgEx;C324R/− (FIG. 17F), and TgEx;R765C/− (FIG. 17G). FIG. 17H show morphometric analysis showing g-ratios of sciatic nerve myelinated axons Cntnap1^{-/-} (control), C324R/−, TgEx;C324R/−, R765C/− and TgEx;R765C/− mice. FIG. 17I and FIG. 17J show TEM images of longitudinal sections of spinal cords at the level of the paranodes from 7-week post-tamoxifen injection TgEx;C324R/− (FIG. 17I), and TgEx;R765C/− (FIG. 17J). Black arrowheads point to paranodal septa. FIG. 17K and FIG. 17L show TEM images of longitudinal sections of sciatic nerves at the level of the paranodes from 7-week post-tamoxifen injection from TgEx;C324R/− (FIG. 17K), and TgEx;R765C/− (FIG. 17L). Black arrowheads point to paranodal septa. FIG. 17M and FIG. 17N show TEM images of longitudinal sections of spinal cords at the level of the paranodes from 7-week post-tamoxifen injection TgEx;C324R/− (FIG. 17M), and TgEx;R765C/− (FIG. 17N). Black arrowheads point to paranodal septa and arrows point to everted loops. FIG. 17O and FIG. 17P show TEM images of longitudinal sections of sciatic nerves at the level of the paranodes from 7-week post-tamoxifen injection from TgEx;C324R/− (FIG. 17O), and TgEx;R765C/− (FIG. 17P). Black arrowheads point to paranodal septa and arrows point to everted loops. Scale bars: (A-G)=2 m; (I-L)=0.2 μm; (M-P)=0.4 μm.

FIGS. 17A-N show Cntnap1^{C324R} and Cntnap1^{R765C} mutant proteins remain in the neuronal soma and shows reduced binding with contactin. FIGS. 17A-D show immunostaining of spinal cord cross sections from +/− (Control, FIG. 17A), KO (FIG. 17B), C324R/− (FIG. 17C) and R765C/− (FIG. 17D) using antibodies against Cntnap1 (Aa-Da, green), Contactin (Ab-Db, red) and NeuN to label neuronal soma (Ac-Dc, blue) and the merged image (Ad-Dd). Scale bar: 50 mm. FIG. 17E is a schematic showing the known protein domains in Cntnap1 and Contactin and their alignment in cis in the cell membrane.

FIG. 17F show co-immunoprecipitation from spinal cord lysates using anti-Contactin antibodies and immunoblotted with anti-Cntnap1 antibodies from +/− (control), C324R/− and R765C/− mutants and TgEx;C324R/− and TgEx;R765/− rescue animals. Total input of Cntnap1 and Contactin protein levels in these animals also are shown here. FIGS. 17G-I) are representative immunostaining images of Cntnap1 and Contactin co-staining in live non-permeable HEK cells co-transfected with Cntnap1 (WT) (FIG. 17G), Cntnap1^{C324R/-} (FIG. 17H) and Cntnap1^{R765C} (FIG. 17I) mutant cDNAs (green) together with Contactin (red). HEK cells were firstly stained with Contactin antibody without the Triton permeabilization; after removing the Contactin antibody, cells were permeabilized with Triton and stained with anti-Cntnap1 antibody. Scale bar: 5 mm. FIG. 17J shows co-immunoprecipitation of Cntnap1 protein and Contactin proteins in HEK cells co-transfected with both Contactin and Cntnap1 (WT), C324R or R765C mutant cDNAs. FIG. 17K shows the cell surface/membrane expression of Cntnap1 and Contactin in HEK cells analyzed by Sulfo-NHS-SS-Biotin assay. HEK cells were transfected with either Cntnap1 (WT), Cntnap1^{C324R} or Cntnap1^{R765C} mutant cDNAs or only Contactin. FIGS. 17L-N show the cell surface/membrane expression of Cntnap1 (WT), C324R or R765C mutant in HEK cells co-transfected with Cntnap1 wt or mutant cDNA and Contactin cDNA) (FIG. 17L). Quantification of relative levels of cell surface expression of Cntnap1 (FIG. 17M) or Contactin (FIG. 17N) in these HEK cells. EGFR was used as the marker for cell surface proteins and GAPDH was used as negative control for cell surface protein expression.

FIG. 18 shows a lentivirus construct map. A human neuron promoter (hSyn) was used to drive the gene expression. A small Flag tag was added to distinguish this wild type Cntnap1 protein from endogenous version of the Cntnap1 protein, and this tag did not affect the overall function of Cntnap1.

FIG. 19 shows a detailed lentivirus construct of the map shown in FIG. 18.

FIGS. 20A-D show the generation of Cntnap1 mouse mutant carrying human CNTNAP1^{G3501V} mutation and their phenotypic analysis. FIG. 20A shows the schematic of the Cntnap1 protein and the approximate locations of amino acid change. Functional domains based on the primary amino acid sequence and homology are indicated. FIG. 20B shows the genomic DNA sequences from wild-type controls Cntnap1^{G350} (GGT) and Cntnap1 mutants Cntnap1^{V350} (GTT). TTAGGGTAATG is SEQ ID NO: 22; and TTAGGTTAATG is SEQ ID NO: 23. FIG. 20C shows the representative immunoblots showing the protein expression of Cntnap1 in the CNS (spinal cords) from Cntnap1^{+/-}, V350/− and KO mice. β-actin was used here as the loading control. FIG. 20D shows immunostaining of sciatic nerves from +/−, G350V/−, and KO mice using antibodies against Kᵥ1.2 (juxtaparanodes, green), Cntnap1 (paranodes, red) and PIV Spectrin (nodes, blue).

FIGS. 21A-C show Cntnap1^{G350/-} mutants display severe motor disability and decline in nerve conduction properties. FIG. 21A shows the body weights of control, G350V- and KO mice from postnatal day 2 weeks to 16 weeks. FIG. 21B shows the beam walking motor coordination performance of +/− (control), G350V/− and KO mice. Walking distances traveled by each mouse (the full length of the beam is 50 cm). FIG. 21C shows representative electrical impulse traces from P20 sciatic nerves and ankle from +/− (control), G350V/− and KO mice.

### SUMMARY

Disclosed herein are transgenic mice comprising a CNTNAP1 mutation.

Disclosed herein are methods of screening for a biologically active agent effective for the treatment of a neuropathy caused by a CNTNAP1 mutation.

Disclosed herein are methods of treating a patient with a neuropathy caused by a CNTNAP1 mutation, the method comprising: a) identifying a patient in need of treatment; and b) administering to the patient a therapeutically effective amount of a lentivirus comprising a nucleic acid construct disclosed herein.

Disclosed herein are transgenic mice comprising a genome, wherein the genome comprises a null mutation in a first copy of the mouse Contactin-associated protein 1 (Cntnap1) gene, and a mutation in a second copy of the mouse Cntnap1 gene, wherein the mutation in the second copy of the mouse Cntanp1 gene is T>C (for C324R), C>T (for R765C) or G>T (for G350V), and is expressed in the transgenic mice.

Disclosed herein are transgenic mice comprising a genome comprising a modified mouse Contactin-associated protein 1 (Cntnap1) gene, wherein the modified mouse Cntnap1 gene comprises a nucleotide modification compared to a wild-type mouse Cntnap1 gene, wherein the nucleic acid modification is a substitution of a thymine for a cytosine at nucleic acid position 4985 (for C324R) of a wild-type Cntnap1 gene of SEQ ID NO: 33, a substitution of a cytosine for thymine (for R765C) at nucleic acid position 8926 of a wild-type Cntnap1 gene of SEQ ID NO: 33, or substitution of a guanine for a thymine (for G350V) at nucleic acid position 5709 of a wild-type CNTNAP1 gene of SEQ ID NO: 33.

Disclosed herein are transgenic mice comprising a genome comprising a modified Contactin-associated protein 1 (CNTNAP1) polypeptide, wherein the modified CNTNAP1 polypeptide comprises an amino acid modification compared to a wild-type CNTNAP1 polypeptide, wherein the amino acid modification is a substitution of a cysteine for a arginine at amino acid position 324 (for C324R) of a wild-type CNTNAP1 polypeptide of Accession ID NP_058062.2, a substitution of a arginine for a cysteine at amino acid position 765 (for R765C) of a wild-type CNTNAP1 polypeptide of Accession ID NP_058062.2, or a substitution of a glycine for a valine at amino acid position 350 (for G350V) of a wild-type CNTNAP1 polypeptide of Accession ID NP_058062.2.

Disclosed herein are polynucleotides comprising an expression cassette, wherein the expression cassette comprises a transcriptional regulatory region comprising a promoter operatively linked to a nucleotide sequence as set forth in SEQ ID NO: 24 encoding the human Contactin-associated protein 1 (hCNTNAP1) protein.

Disclosed herein are methods of treating and/or preventing Contactin-associated protein 1 (hCNTNAP1) protein deficiency in a subject in need thereof, the methods comprising administering to the subject, a pharmaceutical composition comprising a therapeutically effective amount of the vector comprising a polynucleotide comprising an expression cassette, wherein the expression cassette comprises a transcriptional regulatory region comprising a promoter operatively linked to a nucleotide sequence as set forth in SEQ ID NO: 24 encoding the human Contactin-associated protein 1 (hCNTNAP1) protein, and a pharmaceutically acceptable carrier and/or adjuvant.

Disclosed herein are methods of preventing or reducing severe respiratory distress in a subject in need thereof, the methods comprising administering to the subject, a pharmaceutical composition comprising a therapeutically effective amount of the vector comprising a polynucleotide comprising an expression cassette, wherein the expression cassette comprises a transcriptional regulatory region comprising a promoter operatively linked to a nucleotide sequence as set forth in SEQ ID NO: 24 encoding the human Contactin-associated protein 1 (hCNTNAP1) protein, and a pharmaceutically acceptable carrier and/or adjuvant.

Disclosed herein are methods of increasing nerve conduction in a subject in need thereof, the methods comprising administering to the subject, a pharmaceutical composition comprising a therapeutically effective amount of the vector comprising a polynucleotide comprising an expression cassette, wherein the expression cassette comprises a transcriptional regulatory region comprising a promoter operatively linked to a nucleotide sequence as set forth in SEQ ID NO: 24 encoding the human Contactin-associated protein 1 (hCNTNAP1) protein, and a pharmaceutically acceptable carrier and/or adjuvant.

Disclosed herein are methods of reducing ataxia in a subject in need thereof, the methods comprising administering to the subject, a pharmaceutical composition comprising a therapeutically effective amount of the vector comprising a polynucleotide comprising an expression cassette, wherein the expression cassette comprises a transcriptional regulatory region comprising a promoter operatively linked to a nucleotide sequence as set forth in SEQ ID NO: 24 encoding the human Contactin-associated protein 1 (hCNTNAP1) protein, and a pharmaceutically acceptable carrier and/or adjuvant.

Disclosed herein are methods of reducing motor dysfunction in a subject in need thereof, the methods comprising administering to the subject, a pharmaceutical composition comprising a therapeutically effective amount of the vector comprising a polynucleotide comprising an expression cassette, wherein the expression cassette comprises a transcriptional regulatory region comprising a promoter operatively linked to a nucleotide sequence as set forth in SEQ ID NO: 24 encoding the human Contactin-associated protein 1 (hCNTNAP1) protein, and a pharmaceutically acceptable carrier and/or adjuvant.

Disclosed herein are methods of reducing or reversing paralysis in a subject in need thereof, the methods comprising administering to the subject, a pharmaceutical composition comprising a therapeutically effective amount of the vector comprising a polynucleotide comprising an expression cassette, wherein the expression cassette comprises a transcriptional regulatory region comprising a promoter operatively linked to a nucleotide sequence as set forth in SEQ ID NO: 24 encoding the human Contactin-associated protein 1 (hCNTNAP1) protein, and a pharmaceutically acceptable carrier and/or adjuvant.

Disclosed herein are methods of ameliorating a symptom of a CNTNAP1 mutation in subject in need thereof, the methods comprising administering to the subject, a pharmaceutical composition comprising a therapeutically effective amount of the vector comprising a polynucleotide comprising an expression cassette, wherein the expression cassette comprises a transcriptional regulatory region comprising a promoter operatively linked to a nucleotide sequence as set forth in SEQ ID NO: 24 encoding the human Contactin-associated protein 1 (hCNTNAP1) protein, and a pharmaceutically acceptable carrier and/or adjuvant.

Disclosed herein are methods of treating a subject with a neuropathy caused by a CNTNAP1 mutation, the methods comprising administering to the subject, a pharmaceutical composition comprising a therapeutically effective amount of the vector comprising a polynucleotide comprising an expression cassette, wherein the expression cassette comprises a transcriptional regulatory region comprising a promoter operatively linked to a nucleotide sequence as set forth in SEQ ID NO: 24 encoding the human Contactin-associated protein 1 (hCNTNAP1) protein, and a pharmaceutically acceptable carrier and/or adjuvant.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step. The term “patient” can refer to a subject having a CNTNAP1 gene mutation (e.g. a subject having T>C (for C324R), C>T (for R765C) or G>T (for G350V)) described herein, including a subject diagnosed to suffer from a neuropathy caused by a CNTNAP1 gene mutation, but also includes a subject, for example, during or after therapy.

As used herein, the term “comprising” can include the aspects “consisting of” and “consisting essentially of.” “Comprising” can also mean “including but not limited to.” “Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level or animal disease model. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels or animal disease model.

“Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100%, or more, or any amount of promotion in between compared to native or control levels. In an aspect, the increase or promotion is 10-20, 20-30, 30-40, 40-50, 50-60, 60-70, 70-80, 80-90, or 90-100% as compared to native or control levels. In an aspect, the increase or promotion is 0-25, 25-50, 50-75, or 75-100%, or more, such as 200, 300, 500, or 1000% more as compared to native or control levels. In an aspect, the increase or promotion can be greater than 100 percent as compared to native or control levels, such as 100, 150, 200, 250, 300, 350, 400, 450, 500% or more as compared to the native or control levels or animal disease model.

As used herein, the term “determining” can refer to measuring or ascertaining a quantity or an amount or a change in activity. For example, determining the amount of a disclosed polypeptide in a sample as used herein can refer to the steps that the skilled person would take to measure or ascertain some quantifiable value of the polypeptide in the sample. The art is familiar with the ways to measure an amount of the disclosed polypeptides and disclosed nucleotides in a sample.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, affection.

As used herein, the term “normal” refers to an individual, a sample or a subject that does not have a CNTNAP1 gene mutation (e.g., T>C (for C324R), C>T (for R765C) or G>T (for G350V) or does not have a neuropathy caused by a Cntnap1 mutation (e.g., T>C (for C324R), C>T (for R765C) or G>T (for G350V)).

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “expression vector” is herein to refer to vectors that are capable of directing the expression of genes to which they are operatively-linked. Common expression vectors of utility in recombinant DNA techniques are often in the form of plasmids. Recombinant expression vectors can comprise a nucleic acid as disclosed herein in a form suitable for expression of the acid in a host cell. In other words, the recombinant expression vectors can include one or more regulatory elements or promoters, which can be selected based on the host cells used for expression that is operatively linked to the nucleic acid sequence to be expressed.

The term “sequence of interest” or “gene of interest” can mean a nucleic acid sequence (e.g., a therapeutic gene), that is partly or entirely heterologous, i.e., foreign, to a cell into which it is introduced.

The term “sequence of interest” or “gene of interest” can also mean a nucleic acid sequence, that is partly or entirely homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). For example, a sequence of interest can be cDNA, DNA, or mRNA.

The term “sequence of interest” or “gene of interest” can also mean a nucleic acid sequence that is partly or entirely complementary to an endogenous gene of the cell into which it is introduced.

A “sequence of interest” or “gene of interest” can also include one or more transcriptional regulatory sequences and any other nucleic acid, such as introns, that may be necessary for optimal expression of a selected nucleic acid. A “protein of interest” means a peptide or polypeptide sequence that is expressed from a sequence of interest or gene of interest.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the terms “promoter,” “promoter element,” or “promoter sequence” are equivalents and as used herein, refers to a DNA sequence which when operatively linked to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription.

Suitable promoters can be derived from genes of the host cells where expression should occur or from pathogens for this host cells (e.g., tissue promoters or pathogens like viruses). If a promoter is an inducible promoter, then the rate of transcription increases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s) such as leaves, roots or meristem. The term “tissue specific” as it applies to a promoter refers to a promoter that is capable of directing selective expression of a nucleotide sequence or gene of interest to a specific type of tissue in the relative absence of expression of the same nucleotide sequence or gene of interest in a different type of tissue.

The phrase “at least” preceding a series of elements is to be understood to refer to every element in the series. For example, “at least one” includes one, two, three, four or more.

As used herein, the term “transgene” describes genetic material that has been or will be or is about to be inserted into the genome of a cell (e.g., a mammalian cells for implantation into a living animal).

As used herein, the term “transformation” refers to a permanent or transient genetic change induced in a cell following incorporation of exogenous DNA to the cell.

As used herein, the phrase “transgenic animal” refers to a non-human animal, generally, a mammal (e.g., mouse, rat, rabbit, etc.) having a non-endogenous (e.g., heterologous) nucleic acid sequence present as an extrachromosomal element in a portion of its cell or stably integrated into its germ line DNA (e.g., in the genomic sequence of most or all of its cells). The phrase “transgenic animal” also includes the founder transgenic non-human animal and progeny of the founders as well as cells, cell lines and tissues from such animals in which one or more of the cells of the animal includes one or more transgenes.

As used herein, “knock-out” of a gene means an alteration in the sequence of the gene or sequence associated with the gene that results in a decrease of function of the target gene.

For example, the knock-out or ablation of gene can lead the expression of the target gene below detectable levels or where with expression level is present at insignificant levels.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, certain changes and modifications may be practiced within the scope of the appended claims.

Congenital hypomyelination neuropathy is a neurological disorder present at birth.

Symptoms include delayed motor development, muscle weakness, poor muscle tone, impaired coordination, areflexia, impaired somatosensory and somatomotor function, and slowed nerve conduction. Current treatment of congenital hypomyelination neuropathy is symptomatic and supportive (“Neuropathy, Congenital Hypomvelination.” National Organization for Rare Disorders. Accessed: Nov. 27, 2021. [Online]. Available: rarediseases.org/rare-diseases/neuropathy-congenital-hypomyelination/).

Contactin-associated protein 1 (CNTNAP1, also known as CASPR1 or NCP1 (Neurexin IV/Caspr/Paranodin)) is an important constituent of the axo-glial paranodal junctions in myelinated axons. In humans with congenital hypomyelination neuropathy, compound heterozygous mutations in the CNTNAP1 gene have been identified.

Three genetically modified mice have been created that carry specific mutations in the Cntnap1 gene, modeled after human CNTNAP1 gene mutations, which cause congenital hypomyelination neuropathy and generalized paralysis in children. Tools for studying human CNTNAP1 mutation-related disease is limited to the available transgenic mouse lines which can be used for discovery and pre-clinical research, and rescuing the human disease condition. Further, and as described herein, a transgenic mouse line was developed that can overcome the deficits in mutant Cntnap1 mice and provide the proof-of-concept for designing further clinical intervention in humans.

Bidirectional cellular and molecular interactions between neuronal axons and myelin-producing glial cells emerged as the underlying basis for myelination to promote saltatory nerve conduction. A major feature of myelinated axons is their organization into highly specialized anatomical and molecular domains, named the nodes of Ranvier, the paranodes, the juxtaparanodes, and the internodes (Bhat M A. Curr Opin Neurobiol. 2003; 13(5):552-559; Buttermore E D, et al. J Neurosci Res. 2013; 91(5):603-622; Bhat M A, et al. Neuron. 2001; 30(2):369-383; Boyle M E, et al. Neuron. 2001; 30(2):385-397; Sherman D L, et al. Neuron. 2005; 48(5):737-742; Pillai A M, et al. J Neurosci Res. 2009; 87(8):1773-1793; Thaxton C, and Bhat M A. Results Probl Cell Differ. 2009; 48:1-28; Thaxton C, et al. Neuron. 2011; 69(2):244-257; Thaxton C, et al. J Neurosci. 2010; 30(14):4868-4876; Saifetiarova J, et al. J Neurosci. 2018; 38(28):6267-6282; Saifetiarova J, et al. J Neurosci. 2017; 37(10):2524-2538; Dupree J L, et al. J Neurosci. 1998; 18(5):1642-1649; Dupree J L, et al. J Neurocytol. 1998; 27(9):649-659; and Dupree J L, et al. Galactolipids in the formation and function of the myelin sheath. Microsc Res Tech. 1998; 41(5):431-440. Each of these structures is assembled with distinct molecular complexes: the paranodes flanking the nodes of Ranvier are the main interaction regions between the myelinating glial membrane and the axons to form the paranodal axo-glial septate junctions (Bhat M A, et al. Neuron. 2001; 30(2):369-383; Boyle M E, et al. Neuron. 2001; 30(2):385-397; Sherman D L, et al. Neuron. 2005; 48(5):737-742; and Pillai A M, et al. J Neurosci Res. 2009; 87(8):1773-1793). The paranodes also serve as a molecular barrier between the nodal domain and the juxtaparanodal domain as this barrier function is compromised in the Contactin-associated Protein 1 (Cntnap1), Contactin and Neurofascin (NfascNF155) mutants (Bhat M A, et al. Neuron. 2001; 30(2):369-383; Boyle M E, et al. Neuron. 2001; 30(2):385-397; Pillai A M, et al. J Neurosci Res. 2009; 87(8):1773-1793; and Gollan L, et al. J Cell Biol. 2003; 163(6):1213-1218). The disruption of axon-glial interactions at the node/paranodal domains results in severe pathological conditions (Bhat M A, et al. Neuron. 2001; 30(2):369-383; Boyle M E, et al. Neuron. 2001; 30(2):385-397; and Pillai A M, et al. J Neurosci Res. 2009; 87(8):1773-1793). Abnormal paranodal domains have also been reported in human hypomyelination disorders such as Charcot-Marie-Tooth disease (CMT) (Tooth H H. The peroneal type of progressive muscular atrophy. University of Cambridge. 1886; Charcot J M. Rev Med Fr. 1886; 6:97-138; Sahenk Z. Ann N Y Acad Sci. 1999; 883:415-426; and Sahenk Z, and Mendell J R. Alterations in nodes of Ranvier and Schmidt-Lanterman incisures in Charcot-Marie-Tooth neuropathies. Ann N Y Acad Sci. 1999; 883:508-512) and multiple sclerosis (Wolswijk G, and Balesar R. Brain. 2003; 126(Pt 7):1638-1649; and Coman I, et al. Brain. 2006; 129(Pt 12):3186-3195).

Cntnap1 is a transmembrane cell-adhesion protein, which forms a cis interacting complex with axonal paranodal Contactin, a neural cell adhesion molecule, and the 155 kDa isoform of glial paranodal Neurofascin (NF155), an isoform of Neurofascin protein generated in the glial junction (Bonnon C, et al. J Biol Chem. 2003; 278(48):48339-48347; Bonnon C, et al. Mol Biol Cell. 2007; 18(1):229-241; Charles P, et al. Curr Biol. 2002; 12(3):217-220; and Peles E, et al. EMBO J. 1997; 16(5):978-988). Mutant mice that lack the paranodal junction structure display ataxia, motor dysfunction, and severely reduced nerve conduction properties (Bhat M A, et al. Neuron. 2001; 30(2):369-383; Boyle M E, et al. Neuron. 2001; 30(2):385-397; and Pillai A M, et al. J Neurosci Res. 2009; 87(8):1773-1793). Homozygous Cntnap1 null mice display progressive neurologic defects starting in the second week of life, including lack of mobility, tremors, wide-based gait, and generalized motor paresis. Most Cntnap1 null mice die around three weeks of their postnatal life with severely compromised motor functions properties (Bhat M A, et al. Neuron. 2001; 30(2):369-383). Structure-function analyses of Cntnap1, Contactin, and NF155 have revealed distinct domains that are involved in the protein-protein interactions between Cntnap1, Contactin, and NF155 both in in vitro biochemical studies as well as in vivo functional characterization (Thaxton C, et al. J Neurosci. 2010; 30(14):4868-4876; Gollan L, et al. J Cell Biol. 2003; 163(6):1213-1218; Bonnon C, et al. J Biol Chem. 2003; 278(48):48339-48347; and Bonnon C, et al. Mol Biol Cell. 2007; 18(1):229-241). These earlier studies highlighted specific domains that are involved in interactions between these proteins, their association into biochemical complexes as well as their role in paranodal domain organization.

Mutations in human CNTNAP1 were implicated in human neurological diseases that are characterized by polyhydramnios, severe neonatal hypotonia, arthrogryposis, and severe motor paralysis (Laquerriere A, et al. Hum Mol Genet. 2014; 23(9):2279-2289). In subsequent years a large number of CNTNAP1 mutations were identified in over 40 families with subjects carrying frameshift, nonsense and missense mutations (Sabbagh S. et al. Case Rep Med. 2020; 2020:8795607; and Vallat J M, et al. J Neuropathol Exp Neurol. 2016; 75(12):1155-1159). The mutations resulted in a wide range of phenotypes and variable survival rates, ranging from infancy up to early childhood with support (Vallat J M, et al. J Neuropathol Exp Neurol. 2016; 75(12):1155-1159; Mehta P, et al. Muscle Nerve. 2017; 55(5):761-765; Conant A, et al. J Child Neurol. 2018; 33(10):642-650; and Nizon M, et al. Eur J Hum Genet. 2017; 25(1):150-152). The most severe phenotypes resulted in acute respiratory distress and muscle atrophy (Sabbagh S, et al. Case Rep Med. 2020; 2020:8795607). Despite the mounting evidence of CNTNAP1 mutations in human disease, so far there are no mouse models available for studying the impact of Cntnap1 mutations observed in human subjects and the potential mechanisms through which the Cntnap1 mutant proteins alter their functions and lead to neurological deficits.

Described herein are three human CNTNAP1 mutations recapitulated in a mouse model. For this, three single nucleotide substitutions in the Cntnap1 gene (Cntnap1^{C324R}, Cntnap1^{R765C} and Cntnap1^{G350}) were generated. Compared to the control Cntnap1 mice, the Cntnap1^{C324R/-} and Cntnap1^{R765C/-} mutant mice suffered severe weight loss from birth and showed progressive motor and neurological deficits, disorganization of the axo-glial paranodal domains and diminished nerve conduction properties. The Cntnap1^{C324R} and Cntnap1^{R765C/-} proteins were nearly undetectable in the paranodal domains and severely disrupted the paranodal structure and function, as revealed by the mislocalization of the juxtaparanodal proteins to the paranodal areas flanking the node, as was previously reported in the Cntnap1 null mice (Bhat M A, et al. Neuron. 2001; 30(2):369-383). Using in vivo and in vitro biochemical studies, the results show that the Cntnap1^{C324R} and Cntnap1R765C mutant proteins are less stable than the wild-type Cntnap1 and are retained in the cell soma with dramatically reduced cell surface expression as well as binding with Contactin. An inducible Cntnap1 mouse strain that expresses the wild-type Cntnap1 protein in a spatio-temporal manner was also generated. When this wild-type Cntnap1 protein was expressed in Cntnap1^{C324R} and Cntnap1^{R765C} mice, it allowed progressive restoration of the paranodal domains, nerve conduction electrophysiological properties and motor functions. Together, the results described herein demonstrate that Cntnap1^{C324R} and Cntnap1^{R765C} mutations are loss of function mutations that are rescued by the wild-type Cntnap1 protein and expressing human CNTNAP1 using gene therapy methodologies to restore neurological and motor functions in human subjects carrying CNTNAP1 mutations can be useful.

### Transgenic Mice

Disclosed herein is a transgenic mouse and methods of producing a transgenic mouse. Disclosed herein are non-human transgenic animal models useful for screening drugs or candidate drugs. In some aspects, the transgenic mouse comprises a genome wherein one allele of the Cntnap1 gene is dysfunctional or contains a null mutation, and the other allele of the Cntnap1 gene is a transgene expressing one or more mutations. In some aspects, the null mutation can be a deletion of mouse exon 7 and mouse exon 8.

Disclosed herein is a transgenic mouse comprising a genome, wherein the genome comprises a null mutation in a first copy of the mouse Contactin-associated protein 1 (Cntnap1) gene, and a mutation in a second copy of the mouse Cntnap1 gene. In some aspects, the mutation in the second copy of the mouse Cntanp1 gene is T>C (for C324R), C>T (for R765C) or G>T (for G350V), and is expressed in the transgenic mouse.

Disclosed herein is a transgenic mouse comprising a genome comprising a modified mouse Contactin-associated protein 1 (Cntnap1) gene. In some aspects, wherein the modified mouse Cntnap1 gene comprises a nucleotide modification compared to a wild-type mouse Cntnap1 gene, wherein the nucleic acid modification is a substitution of a thymine for a cytosine at nucleic acid position 4958 (for C324R) of a wild-type Cntnap1 gene of SEQ ID NO: 33, a substitution of a cytosine for a thymine (for R765C) at nucleic acid position 8926 of a wild-type Cntnap1 gene of SEQ ID NO: 33, or substitution of a guanine for a thymine (for G350V) at nucleic acid position 5709 of a wild-type CNTNAP1 gene of SEQ ID NO: 33.

Disclosed herein is a transgenic mouse comprising a genome capable of expressing a modified Contactin-associated protein 1 (CNTNAP1) polypeptide. In some aspects, the modified CNTNAP1 polypeptide comprises an amino acid modification compared to a wild-type CNTNAP1 polypeptide, wherein the amino acid modification is a substitution of a cysteine for a arginine at amino acid position 324 (for C324R) of a wild-type CNTNAP1 polypeptide of Accession ID NP_058062.2, a substitution of a arginine for a cysteine at amino acid position 765 (for R765C) of a wild-type CNTNAP1 polypeptide of Accession ID NP_058062.2, or a substitution of a glycine for a valine at amino acid position 350 (for G350V) of a wild-type CNTNAP1 polypeptide of Accession ID NP_058062.2.

In some aspects, the transgenic mouse further comprises a wild-type inducible or regulatable Contactin-associated protein 1 (Cntnap1) gene of gene ID 533211 Accession Number NC_000077.7:101065429-101081550 (SEQ ID NO: 33).

The protein sequence of wild type mouse Cntnap1 gene is:

(SEQ ID NO: 25; Accession ID NP_058062.2)
MMSLRLFSILLATVVSGAWGWGYYGCNEELVGPLYARSLGASSYYGLFTTARFARL
HGISGWSPRIGDPNPWLQIDLMKKHRIRAVATQGAFNSWDWVTRYMLLYGDRVDS
WTPFYQKGHNATFFGNVNDSAVVRHDLHYHFTARYIRIVPLAWNPRGKIGLRLGIY
GCPYTSSILYFDGDDAISYRFQRGASQSLWDVFAFSFKTEEKDGLLLHTEGSQGDYV
TLELQGAHLLLHMSLGSSPIQPRPGHTTVSLGGVLNDLSWHYVRVDRYGRDANFTL
DGYAHHFVLNGDFERLNLENEIFIGGLVGAARKNLAYRHNFRGCIENVIYNRINIAE
MAVMRHSRITFEG
NVAFRCLDPVPHPINFGGPHNFVQVPGFPRRGRLAVSFRFRTWDLTGLLLFSHLGDG
LGHVELMLSEGQVNVSIAQTGRKKLQFAAGYRLNDGFWHEVNFVAQENHAVISIDD
VEGAEVRVSYPLLIRTGTSYFFGGCPKPASRWGCHSNQTAFHGCMELLKVDGQLVN
LTLVEFRKLGYFAEVLFDTCGITDRCSPNMCEHDGRCYQSWDDFICYCELTGYKGVT
CHEPLYKESCEAYRLSGKYSGNYTIDPDGSGPLKPFVVYCDIRENRAWTVVRHDRL
WTTRVTGSSMDRPFLGAIQYWNASWEEVSALANASQHCEQWIEFSCYNSRLLNTAG
GYPYSFWIGRNE
EQHFYWGGSQPGIQRCACGLDQSCVDPALHCNCDADQPQWRTDKGLLTFVDHLPV
TQVVVGDTNRSNSEAQFFLRPLRCYGDRNSWNTISFHTGAALRFPPIRANHSLDVSF
YFRTSAPSGVFLENMGGPFCRWRRPYVRVELNTSRDVVFAFDIGNGDENLTVHSDD
FEFNDDEWHLVRAEINVKQARLRVDHRPWVLRPMPLQTYIWLVYDQPLYVGSAEL
KRRPFVGCLRAMRLNGVTLNLEGRANASEGTFPNCTGHCTHPRFPCFHGGRCVERY
SYYTCDCDLTAFDGPYCNHDIGGFFETGTWMRYNLQSALRSAAREFSHMLSRPVPG
YEPGYVPGYDTPGYVPGYHGPGYRLPEYPRPGRPVPGYRGPVYNVTGEEVSFSFSTN
SAPAVLLYVSSFVRDYMAVLIKEDGTLQLRYQLGTSPYVYQLTTRPVTDGQPHSVNI
TRVYRNLFIQVDYFPLTQKFSLLVDSQLDSPKALYLGRVMETGVIDPEIQRYNTPGFS
GCLSGVRFNNVAPLKTHFRTPRPMTAELAEAMRVQGELSESNCGAMPRLVSEVPPE
LDPWYLPPDFPYYHDDGWIAILLGFLVAFLLLGLVGMLVLFYLQNHRYKGSYHTNE
PKATHDSHPGGKAPLPPSGPAQAPAPTPAPTQLPTPAPAPAPAPASGPGPRDQNLPQI
LEESRSE. Cys324, Gly350 and Arg765 are in bold and underline.

An example of an amino acid sequence with a C324R mutation can be the sequence of

(SEQ ID NO: 26)
MMSLRLFSILLATVVSGAWGWGYYGCNEELVGPLYARSLGASSYYGLFTTARFARL
HGISGWSPRIGDPNPWLQIDLMKKHRIRAVATQGAFNSWDWVTRYMLLYGDRVDS
WTPFYQKGHNATFFGNVNDSAVVRHDLHYHFTARYIRIVPLAWNPRGKIGLRLGIY
GCPYTSSILYFDGDDAISYRFQRGASQSLWDVFAFSFKTEEKDGLLLHTEGSQGDYV
TLELQGAHLLLHMSLGSSPIQPRPGHTTVSLGGVLNDLSWHYVRVDRYGRDANFTL
DGYAHHFVLNGDFERLNLENEIFIGGLVGAARKNLAYRHNFRGRIENVIYNRINIAE
MAVMRHSRITFEG
NVAFRCLDPVPHPINFGGPHNFVQVPGFPRRGRLAVSFRFRTWDLTGLLLFSHLGDG
LGHVELMLSEGQVNVSIAQTGRKKLQFAAGYRLNDGFWHEVNFVAQENHAVISIDD
VEGAEVRVSYPLLIRTGTSYFFGGCPKPASRWGCHSNQTAFHGCMELLKVDGQLVN
LTLVEFRKLGYFAEVLFDTCGITDRCSPNMCEHDGRCYQSWDDFICYCELTGYKGVT
CHEPLYKESCEAYRLSGKYSGNYTIDPDGSGPLKPFVVYCDIRENRAWTVVRHDRL
WTTRVTGSSMDRPFLGAIQYWNASWEEVSALANASQHCEQWIEFSCYNSRLLNTAG
GYPYSFWIGRNE
EQHFYWGGSQPGIQRCACGLDQSCVDPALHCNCDADQPQWRTDKGLLTFVDHLPV
TQVVVGDTNRSNSEAQFFLRPLRCYGDRNSWNTISFHTGAALRFPPIRANHSLDVSF
YFRTSAPSGVFLENMGGPFCRWRRPYVRVELNTSRDVVFAFDIGNGDENLTVHSDD
FEFNDDEWHLVRAEINVKQARLRVDHRPWVLRPMPLQTYIWLVYDQPLYVGSAEL
KRRPFVGCLRAMRLNGVTLNLEGRANASEGTFPNCTGHCTHPRFPCFHGGRCVERY
SYYTCDCDLTAFDGPYCNHDIGGFFETGTWMRYNLQSALRSAAREFSHMLSRPVPG
YEPGYVPGYDTPGYVPGYHGPGYRLPEYPRPGRPVPGYRGPVYNVTGEEVSFSFSTN
SAPAVLLYVSSFVRDYMAVLIKEDGTLQLRYQLGTSPYVYQLTTRPVTDGQPHSVNI
TRVYRNLFIQVDYFPLTEQKFSLLVDSQLDSPKALYLGRVMETGVIDPEIQRYNTPGF
SGCLSGVRFNNVAPLKTHFRTPRPMTAELAEAMRVQGELSESNCGAMPRLVSEVPPE
LDPWYLPPDFPYYHDDGWIAILLGFLVAFLLLGLVGMLVLFYLQNHRYKGSYHTNE
PKATHDSHPGGKAPLPPSGPAQAPAPTPAPTQLPTPAPAPAPAPASGPGPRDQNLPQI
LEESRSE.

An example of an amino acid sequence with a G350V mutation can be the sequence of

(SEQ ID NO: 27)
MMSLRLFSILLATVVSGAWGWGYYGCNEELVGPLYARSLGASSYYGLFTTARFARL
HGISGWSPRIGDPNPWLQIDLMKKHRIRAVATQGAFNSWDWVTRYMLLYGDRVDS
WTPFYQKGHNATFFGNVNDSAVVRHDLHYHFTARYIRIVPLAWNPRGKIGLRLGIY
GCPYTSSILYFDGDDAISYRFQRGASQSLWDVFAFSFKTEEKDGLLLHTEGSQGDYV
TLELQGAHLLLHMSLGSSPIQPRPGHTTVSLGGVLNDLSWHYVRVDRYGRDANFTL
DGYAHHFVLNGDFERLNLENEIFIGGLVGAARKNLAYRHNFRGCIENVIYNRINIAE
MAVMRHSRITFEV
NVAFRCLDPVPHPINFGGPHNFVQVPGFPRRGRLAVSFRFRTWDLTGLLLFSHLGDG
LGHVELMLSEGQVNVSIAQTGRKKLQFAAGYRLNDGFWHEVNFVAQENHAVISIDD
VEGAEVRVSYPLLIRTGTSYFFGGCPKPASRWGCHSNQTAFHGCMELLKVDGQLVN
LTLVEFRKLGYFAEVLFDTCGITDRCSPNMCEHDGRCYQSWDDFICYCELTGYKGVT
CHEPLYKESCEAYRLSGKYSGNYTIDPDGSGPLKPFVVYCDIRENRAWTVVRHDRL
WTTRVTGSSMDRPFLGAIQYWNASWEEVSALANASQHCEQWIEFSCYNSRLLNTAG
GYPYSFWIGRNE
EQHFYWGGSQPGIQRCACGLDQSCVDPALHCNCDADQPQWRTDKGLLTFVDHLPV
TQVVVGDTNRSNSEAQFFLRPLRCYGDRNSWNTISFHTGAALRFPPIRANHSLDVSF
YFRTSAPSGVFLENMGGPFCRWRRPYVRVELNTSRDVVFAFDIGNGDENLTVHSDD
FEFNDDEWHLVRAEINVKQARLRVDHRPWVLRPMPLQTYIWLVYDQPLYVGSAEL
KRRPFVGCLRAMRLNGVTLNLEGRANASEGTFPNCTGHCTHPRFPCFHGGRCVERY
SYYTCDCDLTAFDGPYCNHDIGGFFETGTWMRYNLQSALRSAAREFSHMLSRPVPG
YEPGYVPGYDTPGYVPGYHGPGYRLPEYPRPGRPVPGYRGPVYNVTGEEVSFSFSTN
SAPAVLLYVSSFVRDYMAVLIKEDGTLQLRYQLGTSPYVYQLTTRPVTDGQPHSVNI
TRVYRNLFIQVDYFPLTEQKFSLLVDSQLDSPKALYLGRVMETGVIDPEIQRYNTPGF
SGCLSGVRFNNVAPLKTHFRTPRPMTAELAEAMRVQGELSESNCGAMPRLVSEVPPE
LDPWYLPPDFPYYHDDGWIAILLGFLVAFLLLGLVGMLVLFYLQNHRYKGSYHTNE
PKATHDSHPGGKAPLPPSGPAQAPAPTPAPTQLPTPAPAPAPAPASGPGPRDQNLPQI
LEESRSE.

An example of an amino acid sequence with a R765C mutation can be the sequence of

(SEQ ID NO: 28)
MMSLRLFSILLATVVSGAWGWGYYGCNEELVGPLYARSLGASSYYGLFTTARFARL
HGISGWSPRIGDPNPWLQIDLMKKHRIRAVATQGAFNSWDWVTRYMLLYGDRVDS
WTPFYQKGHNATFFGNVNDSAVVRHDLHYHFTARYIRIVPLAWNPRGKIGLRLGIY
GCPYTSSILYFDGDDAISYRFQRGASQSLWDVFAFSFKTEEKDGLLLHTEGSQGDYV
TLELQGAHLLLHMSLGSSPIQPRPGHTTVSLGGVLNDLSWHYVRVDRYGRDANFTL
DGYAHHFVLNGDFERLNLENEIFIGGLVGAARKNLAYRHNFRGCIENVIYNRINIAE
MAVMRHSRITFEG
NVAFRCLDPVPHPINFGGPHNFVQVPGFPRRGRLAVSFRFRTWDLTGLLLFSHLGDG
LGHVELMLSEGQVNVSIAQTGRKKLQFAAGYRLNDGFWHEVNFVAQENHAVISIDD
VEGAEVRVSYPLLIRTGTSYFFGGCPKPASRWGCHSNQTAFHGCMELLKVDGQLVN
LTLVEFRKLGYFAEVLFDTCGITDRCSPNMCEHDGRCYQSWDDFICYCELTGYKGVT
CHEPLYKESCEAYRLSGKYSGNYTIDPDGSGPLKPFVVYCDIRENRAWTVVRHDRL
WTTRVTGSSMDRPFLGAIQYWNASWEEVSALANASQHCEQWIEFSCYNSRLLNTAG
GYPYSFWIGRNE
EQHFYWGGSQPGIQRCACGLDQSCVDPALHCNCDADQPQWRTDKGLLTFVDHLPV
TQVVVGDTNCSNSEAQFFLRPLRCYGDRNSWNTISFHTGAALRFPPIRANHSLDVSF
YFRTSAPSGVFLENMGGPFCRWRRPYVRVELNTSRDVVFAFDIGNGDENLTVHSDD
FEFNDDEWHLVRAEINVKQARLRVDHRPWVLRPMPLQTYIWLVYDQPLYVGSAEL
KRRPFVGCLRAMRLNGVTLNLEGRANASEGTFPNCTGHCTHPRFPCFHGGRCVERY
SYYTCDCDLTAFDGPYCNHDIGGFFETGTWMRYNLQSALRSAAREFSHMLSRPVPG
YEPGYVPGYDTPGYVPGYHGPGYRLPEYPRPGRPVPGYRGPVYNVTGEEVSFSFSTN
SAPAVLLYVSSFVRDYMAVLIKEDGTLQLRYQLGTSPYVYQLTTRPVTDGQPHSVNI
TRVYRNLFIQVDYFPLTEQKFSLLVDSQLDSPKALYLGRVMETGVIDPEIQRYNTPGF
SGCLSGVRFNNVAPLKTHFRTPRPMTAELAEAMRVQGELSESNCGAMPRLVSEVPPE
LDPWYLPPDFPYYHDDGWIAILLGFLVAFLLLGLVGMLVLFYLQNHRYKGSYHTNE
PKATHDSHPGGKAPLPPSGPAQAPAPTPAPTQLPTPAPAPAPAPASGPGPRDQNLPQI
LEESRSE.

In some aspects, the transgenic mice disclosed herein can be heterozygous or homozygous (having one or two mutated alleles) for a modified mouse Contactin-associated protein 1 (Cntnap1) gene.

In some aspects, the transgenic mouse displays hypomyelination, an increased g-ratio, or a combination thereof. In some aspects, the transgenic mouse displays weight loss, reduced nerve conduction, progressive motor dysfunction, severe ataxia, paralysis or a combination thereof associated with paranodal axonal domain disorganization, CNTNAP1-associated congenital hypomyelinating neuropathy or a combination thereof.

A transgene can be used to transform a cell so that a genetic change can be present in the induced cell following incorporation of exogenous DNA. A permanent genetic change can be induced in a cell following incorporation of exogenous DNA, for example, into the genome of the cell. Vectors for stable integration include but are not limited to plasmids, retroviruses, other animal viruses, etc.

In some aspects, the modified CNTNAP1 gene can be a mouse gene. In some aspects, the modified CNTNAP1 gene can be a human gene. In some aspects, the modified CNTNAP1 gene can also be a wild-type gene or a genetically manipulated sequence, for example, having deletions, substitutions or insertions in the coding or non-coding regions. The sequence introduced can encode a CNTNAP1 protein or can utilize a human neuron promoter operably linked to a reporter gene. When the introduced gene is a coding sequence, it can be operably linked to promoter that can be constitutive or inducible, and other regulatory sequences required for expression into the host animal. In some aspects, the human neuron promoter can by hSnv. In some aspects, the human promoter can have the nucleic acid sequence of

(SEQ ID NO: 29)
aagtgggttttaggaccaggatgaggcggggtgggggtgcctacctgac
gaccgaccccgacccactggacaagcacccaacccccattccccaaatt
gcgcatcccctatcagagagggggaggggaaacaggatgcggcgaggcg
cgtgcgcactgccagcttcagcaccgcggacagtgccttcgcccccgcc
tggcggcgcgcgccaccgccgcctcagcactgaaggcgcgctgacgtca
ctcgccggtcccccgcaaactccccttcccggccaccttggtcgcgtcc
gcgccgccgccggcccagccggaccgcaccacgcgaggcg.

In some aspects, the Cntnap1gene can be a mutant Cntnap1 gene. In some aspects, the mutant Cntnap1 gene is capable of encoding a modified Contactin-associated protein 1 (CNTNAP1) polypeptide wherein a cysteine to arginine substitution is at amino acid 324 (SEQ ID NO: 26). In some aspects, the mutant Cntnap1 gene is capable of encoding a modified Contactin-associated protein 1 (CNTNAP1) polypeptide wherein a glycine to valine substitution is at amino acid 350 (SEQ ID NO: 27). In some aspects, the mutant Cntnap1 gene is capable of encoding a modified Contactin-associated protein 1 (CNTNAP1) polypeptide wherein an arginine to cysteine substitution is at amino acid 765 (SEQ ID NO: 28). In some aspects, cre-recombinase can regulate Cntnap1A76C5 allele, Cntnap1C323A allele or Cntnap1G350V allele.

DNA constructs for homologous recombination can comprise at least a portion of the Cntnap1 gene with the desired genetic modification and can include regions of homology to the target locus. DNA constructs for random integration do not need to include regions of homology to mediate recombination. Methods for generating cells having targeted gene modifications through homologous recombination are known in the art.

In general, a transgenic animal is produced by the integration of a given transgene into the genome in a manner that permits the expression of the transgene. Methods for producing transgenic animals are known in the art.

In some aspects, the transgenic mouse disclosed herein can serve as a model of human human CNTNAP1 mutation-related disease. In some aspect, the transgenic mouse model disclosed herein can be used as a model of congenital hypomyelinating neuropathy type 3 (CHN3), characterized by severe neonatal hypotonia, polyhydramnios, arthrogryposis, facial diplegia, and severe motor paralysis, leading to death in early infancy.

Disclosed herein are cells, cell lines or primary cell cultures. In some aspects, the cells, cell lines, or primary cell cultures can be derived from the transgenic mouse described herein. In some aspects, cells or cell lines can be isolated from the transgenic animal. In some aspects, the cells or primary cell cultures can be mammalian cells (e.g., mouse cell lines) that harbor an exogenous CNTNAP1 mutated gene. In some aspects, the cell can be a neuron. In some aspects, the CNTNAP1 gene can be a mutant or modified CNTNAP1 gene. In some aspects, the modified CNTNAP1 gene comprises a T>C (for C324R) mutation, C>T (for R765C) mutation, or G>T (for G350V) mutation. In some aspects, the CNTNAP1 gene can be a wild-type gene that is inducible or regulatable. In some aspects, the CNTNAP1 gene can be a null or dysfunctional gene, wherein the null mutation is a deletion of mouse exon 7 and exon 8. In some aspects, the cells or cell lines derived from the transgenic mice disclosed herein can be used for any in vitro characterization for treating the human disease.

The cells lines described herein can be used for a variety of purposes including, but not limited to surveying human tissue and the like.

Also disclosed herein are embryos that are offspring of any of the transgenic mice disclosed herein. In some aspects, the embryo can be heterozygous for the modified Contactin-associated protein 1 (CNTNAP1) gene.

### Compositions

Nucleic acids and polynucleotides. Disclosed herein are nucleic acids comprising at least one transgene operably linked to a promoter, wherein the transgene encodes CNTNAP1 (NC 000017.11:42682531-42699993 CNTNAP1 [GeneID=8506]; SEQ ID NO: 24). Also disclosed herein are polynucleotides comprising a transcriptional regulatory region comprising a promoter operatively linked to a nucleotide sequence encoding the human (hCNTNAP1) protein (SEQ ID NO: 31; NP_003623.1 CNTNAP1 [GeneID=8506]).

In some aspects, the amino acid sequence of the human CNTNAP1 protein is

(SEQ ID NO: 31)
MMHLRLFCILLAAVSGAEGWGYYGCDEELVGPLYARSLGASSYYSLLTAPRFARLH
GISGWSPRIGDPNPWLQIDLMKKHRIRAVATQGSFNSWDWVTRYMLLYGDRVDSW
TPFYQRGHNSTFFGNVNESAVVRHDLHFHFTARYIRIVPLAWNPRGKIGLRLGLYGC
PYKADILYFDGDDAISYRFPRGVSRSLWDVFAFSFKTEEKDGLLLHAEGAQGDYVTL
ELEGAHLLLHMSLGSSPIQPRPGHTTVSAGGVLNDQHWHYVRVDRFGRDVNFTLDG
YVQRFILNGDFERLNLDTEMFIGGLVGAARKNLAYRHNFRGCIENVIFNRVNIADLA
VRRHSRITFEGK
VAFRCLDPVPHPINFGGPHNFVQVPGFPRRGRLAVSFRFRTWDLTGLLLFSRLGDGL
GHVELTLSEGQVNVSIAQSGRKKLQFAAGYRLNDGFWHEVNFVAQENHAVISIDDV
EGAEVRVSYPLLIRTGTSYFFGGCPKPASRWDCHSNQTAFHGCMELLKVDGQLVNL
TLVEGRRLGFYAEVLFDTCGITDRCSPNMCEHDGRCYQSWDDFICYCELTGYKGET
CHTPLYKESCEAYRLSGKTSGNFTIDPDGSGPLKPFVVYCDIRENRAWTVVRHDRLW
TTRVTGSSMERPFLGAIQYWNASWEEVSALANASQHCEQWIEFSCYNSRLLNTAGG
YPYSFWIGRNEE
QHFYWGGSQPGIQRCACGLDRSCVDPALYCNCDADQPQWRTDKGLLTFVDHLPVT
QVVIGDTNRSTSEAQFFLRPLRCYGDRNSWNTISFHTGAALRFPPIRANHSLDVSFYF
RTSAPSGVFLENMGGPYCQWRRPYVRVELNTSRDVVFAFDVGNGDENLTVHSDDFE
FNDDEWHLVRAEINVKQARLRVDHRPWVLRPMPLQTYIWMEYDQPLYVGSAELKR
RPFVGCLRAMRLNGVTLNLEGRANASEGTSPNCTGHCAHPRLPCFHGGRCVERYSY
YTCDCDLTAFDGPYCNHDIGGFFEPGTWMRYNLQSALRSAAREFSHMLSRPVPGYE
PGYIPGYDTPGYVPGYHGPGYRLPDYPRPGRPVPGYRGPVYNVTGEEVSFSFSTSSAP
AVLLYVSSFVRDYMAVLIKDDGTLQLRYQLGTSPYVYQLTTRPVTDGQPHSINITRV
YRNLFIQVDYFPLTEQKFSLLVDSQLDSPKALYLGRVMETGVIDPEIQRYNTPGFSGC
LSGVRFNNVAPLKTHFRTPRPMTAELAEALRVQGELSESNCGAMPRLVSEVPPELDP
WYLPPDFPYYHDEGWVAILLGFLVAFLLLGLVGMLVLFYLQNHRYKGSYHTNEPKA
AHEYHPGSKPPLPTSGPAQVPTPTAAPNQAPASAPAPAPTPAPAPGPRDQNLPQILEES
RSE.

As disclosed herein, the CNTNAP1 gene can encode an mRNA or cDNA having the nucleotide sequence of SEQ ID NO: 32. The CNTNAP1 gene can encode a protein having the amino acid sequence SEQ ID NO: 31. In some aspects, the CNTNAP1 gene is codon-optimized, for example, for expression in a mammal, such as a human. Sequences corresponding to all GenBank accession numbers described in the disclosure are incorporated herein by reference in their entirety. Note that DNA sequences provided herein may also include the reverse complement to form the double stranded DNA sequence or may be a reverse complement of the sequences disclosed herein.

In some aspects, the mRNA or cDNA sequence of the human CNTNAP1 gene is NM_003632.3 CNTNAP1 [GeneID=8506](SEQ ID NO: 32).

In some aspects, the nucleic acid sequence encoding a CNTNAP1 polypeptide can be SEQ ID NO: 24. In some aspects, the nucleic acid sequence encoding a CNTNAP1 polypeptide comprises at least 80%, at least 85%, at least 90%, at least 95%, at least 96%, at least 97%, at least 98%, or at least 99% identity to SEQ ID NO: 24. In some aspects, the nucleic acid sequence encoding CNTNAP1 gene comprises up to 20 nucleotides that are different from the CNTNAP1 gene set forth in SEQ ID NO: 24. In some aspects, the CNTNAP1 gene comprises 1, 2, 3, 4, 5, 6, 7, 8, 9, 10, 11, 12, 13, 14, 15, 16, 17, 18, 19, or 20 nucleotides that are different from the CNTNAP1 gene set forth in SEQ ID NO: 24. In some aspects, the nucleic acid sequence encoding CNTNAP1 gene comprises more than 20 nucleotides that are different from the CNTNAP1 gene set forth in SEQ ID NO: 24.

In some aspects, the nucleic acid sequence encoding CNTNAP1 comprises insertions relative to SEQ ID NO: 24. In some aspects, the nucleic acid sequences encoding CNTNAP1 comprises insertions relative to SEQ ID NO: 24 that do not introduce a frameshift mutation. In some aspects, an insertion in the nucleic acid sequence relative to SEQ ID NO: 24 involves the insertion of multiples of 3 nucleotides (e.g., 3, 6, 9, 12, 15, 18, etc.). In some aspects, an insertion in the nucleic acid sequence relative to SEQ ID NO: 24 leads to an increase in the total number of amino acid residues in the resultant CNTNAP1 protein (e.g., an increase of 1-3, 15, 3-10, 5-10, 5-15, or 10-20 amino acid residues).

In some aspects, the nucleic acid sequence encoding CNTNAP1 comprises deletions relative to SEQ ID NO: 24. In some aspects, the nucleic acid sequences encoding CNTNAP1 comprises deletions relative to SEQ ID NO: 24 that do not introduce a frameshift mutation. In some aspects, a deletion in the nucleic acid sequence relative to SEQ ID NO: 24 involves the deletion of multiples of 3 nucleotides (e.g., 3, 6, 9, 12, 15, 18, etc.). In some aspects, a deletion in the nucleic acid sequence relative to SEQ ID NO: 24 leads to an decrease in the total number of amino acid residues in the resultant PGM1 protein (e.g., a decrease of 1-3, 1-5, 3-10, 5-10, 5-15, or 10-20 amino acid residues). In some aspects, the nucleic acid sequence encoding SEQ ID NO: 24 can be a codon-optimized sequence (e.g., codon optimized for expression in mammalian cells). In some aspects, a codon-optimized sequence encoding CNTNAP1 comprises reduced GC content relative to a wild-type sequence that has not been codon-optimized. In some aspects, a codon-optimized sequence encoding CNTNAP1 comprises a 1-5%, 3-5%, 3-10%, 5-10%, 5-15%, 10-20%, 15-30%, 20-40%, 25-50%, or 30-60% reduction in GC content relative to a wild-type sequence that has not been codon-optimized. In some aspects, a codon-optimized sequence encoding CNTNAP1 comprises fewer guanine and/or cytosine nucleobases relative to a wild-type sequence that has not been codon-optimized. In some aspects, a codon-optimized sequence encoding CNTNAP1 comprises 1-5, 3-5, 3-10, 5-10, 5-15, 10-20, 15-30, 20-40, 25-50, or 30-60 fewer guanine and/or cytosine nucleobases relative to a wild-type sequence that has not been codon-optimized. In some aspects, a codon-optimized sequence encoding CNTNAP1 comprises fewer CpG dinucleotide islands relative to a wild-type sequence that has not been codon-optimized. In some aspects, a codon-optimized sequence encoding CNTNAP1 comprises 1-3, 3-5, 3-10, 5-10, 5-15, 10-20, 15-30, 20-40, 25-50, or 30-60 fewer CpG dinucleotide islands relative to a wild-type sequence that has not been codon-optimized. In some aspects, the nucleotide sequence encoding CNTNAP1 is SEQ ID NO: 24.

Promoters. In the constructs disclosed herein nucleic acid encoding the CNTNAP1 polypeptide, including, the nucleotide sequence of SEQ ID NO: 24, can be operably linked to a promoter to direct expression of the PGM1 coding sequence, particularly in cardiac muscle cells. In some aspects, the promoter can be a constitutive promoter. In some aspects, the promoter can be a constitutive promoter, for example a CAG promoter, a chicken beta-actin (CBA) promoter, a retroviral Rous sarcoma virus (RSV) LTR promoter (optionally with the RSV enhancer), a cytomegalovirus (CMV) promoter (optionally with the CMV enhancer) [see, e.g., Boshart et al., Cell, 41:521-530 (1985)], a CMV enhanced chicken β-actin promoter (CB), a SV40 promoter, a dihydrofolate reductase promoter, a (3-actin promoter, a phosphoglycerol kinase (PGK) promoter, or an EFla promoter [Invitrogen]. In some aspects, a promoter can be a CAG promoter. In some aspects, a promoter can be an enhanced chicken β-actin promoter. In some aspects, a promoter can be a U6 promoter. In some aspects, the promoter can be a CB6 promoter. In some aspects, the promoter can be a JeT promoter. In some aspects, a promoter can be a CB promoter. In some aspects, the promoter can be a human neuron promoter. In some aspects, the human neuron promoter has the sequence of SEQ ID NO: 29.

In some aspects, the sequence of the human neuron promoter is

(SEQ ID NO: 29)
aagtgggttttaggaccaggatgaggcggggtgggggtgcctacctgac
gaccgaccccgacccactggacaagcacccaacccccattccccaaatt
gcgcatcccctatcagagagggggaggggaaacaggatgcggcgaggcg
cgtgcgcactgccagcttcagcaccgcggacagtgccttcgcccccgcc
tggcggcgcgcgccaccgccgcctcagcactgaaggcgcgctgacgtca
ctcgccggtcccccgcaaactccccttcccggccaccttggtcgcgtcc
gcgccgccgccggcccagccggaccgcaccacgcgaggcg

In some aspects, the nucleic acid constructs disclosed herein can further comprise a promoter. The promoter can be any promoter. The promoter can be ubiquitous or cell type specific as the splicing regulation is independent of the promoter. For example, a ubiquitous promoter with a neuron-specific exon sequence can be used to drive gene expression only in neurons. In some aspects, the promoter can be operatively linked to 5′UTR. In some aspects, the promoter can be operatively linked to a start codon. In some aspects, the promoter can be regulatable. In some aspects, the promoter can be constitutively active.

As used herein, the term “promoter” refers to regulatory elements, promoters, promoter enhancers, internal ribosomal entry sites (IRES) and other elements that are capable of controlling expression (e.g., transcription termination signals, including but not limited to polyadenylation signals and poly-U sequences). Promoters can direct constitutive expression.

Promoters can also direct expression in a temporal-dependent manner including but not limited to cell-cycle dependent or developmental stage-dependent. Examples of promoters include but are not limited to WPRE, CMV enhancers, and SV40 enhancers. Specific gene specific promoters can be used. Such promoters allow cell specific expression or expression tied to specific pathways. Any promoter that is active in mammalian cells can be used. In some aspects, the promoter is an inducible promoter including, but not limited to, Tet-on and Tet-off systems. Such inducible promoters can be used to control the timing of the desired expression. In some aspects, the promoter can be an inducible promoter. Examples of inducible promoters include but are not limited to tetracycline inducible system (tet); heat shock promoters and IPTG activated promoters. In some aspects, promoters are bidirectional.

In some aspects, a promoter can be an inducible promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which can be useful include those which are regulated by a specific physiological state, e.g., temperature, acute phase, a particular differentiation state of the cell, or in replicating cells.

In some aspects, the native promoter for the transgene (e.g., CNTNAP1) can be used. In some aspects, the native promoter can be used when it is desired that expression of the transgene should mimic the expression of a native wild-type CNTNAP1 gene (e.g., a non-mutated CNTNAP1 gene). The native promoter can be used when expression of the transgene must be regulated temporally or developmentally, or in a tissue-specific manner, or in response to specific transcriptional stimuli. In some aspects, other native expression control elements, such as enhancer elements or polyadenylation sites can also be used to mimic the native expression. In some aspects, the promoter can drive transgene expression in a non-tissue specific manner. In some aspects, the promoter can drives transgene expression in a specific tissue. In some aspects, the promoter drives transgene expression in brain and/or spinal cord tissues (e.g., neurons).

Cells. Disclosed herein are cells comprising any of the nucleic acid constructs described herein. In some aspects, the nucleic acid constructs as described herein can be delivered to a cell of a subject.

Disclosed herein are transfected host cells. The term “transfection” is used to refer to the uptake of foreign DNA by a cell, and a cell has been “transfected” when exogenous DNA has been introduced through the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the term “host cell” refers to any cell that harbors, or is capable of harboring, a substance of interest. Often a host cell can be a mammalian cell (e.g., a non-human primate, rodent, or human cell). In some aspects, the host cell can be a mammalian cell, a yeast cell, a bacterial cell, an insect cell, a plant cell, or a fungal cell. A host cell can be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein can refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the term “recombinant cell” refers to a cell into which an exogenous DNA segment, such as DNA segment that leads to the transcription of a biologically-active polypeptide or production of a biologically active nucleic acid such as an RNA, has been introduced.

Vectors. Disclosed herein are vectors comprising any of the nucleic acid constructs described herein. Vectors comprising nucleic acids or polynucleotides as described herein are also provided. As used herein, a “vector” refers a carrier molecule into which another DNA segment can be inserted to initiate replication of the inserted segment. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, and viruses (e.g., bacteriophage, animal viruses, and plant viruses), and artificial chromosomes (e.g., YACs). Vectors can comprise targeting molecules. A targeting molecule is one that directs the desired nucleic acid to a particular organ, tissue, cell, or other location in a subject's body. A vector, generally, brings about replication when it is associated with the proper control elements (e.g., a promoter, a stop codon, and a polvadenylation signal). Examples of vectors that are routinely used in the art include plasmids and viruses. The term “vector” includes expression vectors and refers to a vector containing a nucleic acid sequence coding for at least part of a gene product capable of being transcribed. A variety of ways can be used to introduce an expression vector into cells. In some aspects, the expression vector comprises a virus or an engineered vector derived from a viral genome. As used herein, “expression vector” is a vector that includes a regulatory region. A variety of host/expression vector combinations can be used to express the nucleic acid sequences disclosed herein. Examples of expression vectors include but are not limited to plasmids and viral vectors derived from, for example, bacteriophages, retroviruses (e.g., lentiviruses), and other viruses (e.g., adenoviruses, poxviruses, herpesviruses and adeno-associated viruses). Vectors and expression systems are commercially available and known to one skilled in the art.

Vectors for stable integration include but are not limited to plasmids, retroviruses, other animal viruses, etc.

The vectors disclosed herein can also include detectable label or selectable marker or label. A detectable marker or label can be introduced into the locus, where upregulation of expression can result in a detected change in the phenotype. Any of the vectors disclosed herein can also include a detectable marker or label. Such detectable labels can include a tag sequence designed for detection (e.g., purification or localization) of an expressed polypeptide. Tag sequences include, for example, green fluorescent protein, glutathione S-transferase, polyhistidine, c-myc, hemagglutinin, or Flag™ tag, and can be fused with the encoded polypeptide and inserted anywhere within the polypeptide, including at either the carboxyl or amino terminus. The label can comprise any detectable moiety, including, for example, fluorescent labels, radioactive labels, and electronic labels.

In some aspects, the vectors disclosed herein can further comprise Flag and Myc dual-tag tail sequence. The Flag and Myc dual-ta can distinguish the expression of the exogenous Cntnap1 from endogenous Cntap1. An example of a Flag and Myc dual-tag tail sequence is:

(SEQ ID NO: 30)
gagcagaaactcatctcagaagaggatctgcgtacgcggccgctcgatt
acaaggatgacgacgataag.

In some aspects, the vector can further comprise conventional control elements which are operably linked with elements of the transgene in a manner that permits its transcription, translation and/or expression in a cell transfected with the vector or infected with the virus produced by the disclosure. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

The term “expression cassette” as used herein refers to a nucleic acid construct. The expression cassette can be produced either through recombinant techniques or synthetically that will result in the transcription of a certain polynucleotide sequence in a host cell. The expression cassette can be part of a plasmid, viral genome or nucleic acid fragment. Generally, the expression cassette includes a polynucleotide operably linked to a promoter. In some aspects, the expression cassette can be a plasmid. The expression cassette can be adapted for expression in a specific type of host cell (e.g., using a cell specific exon sequence). The expression cassette can also comprise other components such as polyadenylation signals, enhancer elements or any other component that results in the expression of the nucleic acid constructs disclosed herein in a specific type of host cell.

Vectors include, for example, viral vectors (such as adenoviruses (“Ad”), adeno-associated viruses (AAV), and retroviruses, including lentiviruses), liposomes and other lipid-containing complexes, and other macromolecular complexes capable of mediating delivery of a polynucleotide to a host cell. Vectors can also comprise other components to further modulate the delivery and/or expression of the gene of interest, for example, or that otherwise provides beneficial properties to the targeted cells. A wide variety of vectors is known to those skilled in the art and is generally available. Other suitable complexes capable of mediating delivery of any of the nucleic acid constructs described herein include retroviruses (e.g., lentivirus), vaults, cell penetrating peptides and biolistic particle guns. Cell penetrating peptides are capable of transporting or translocating proteins across a plasma membrane; thus, cell penetrating peptides act as delivery vehicles. Examples include but are not limited to labels (e.g., GFP, MRI contrast agents, quantum dots).

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of Translational Medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic.

Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56.degree. C. to 65.degree. C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2: 619-623 (2000) and Chao et al., Mol Ther, 4: 217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-(1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-(1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector; as such a vector is contained within an AAV vector particle.

Recombinant AAV genomes of the invention comprise nucleic acid molecule of the invention and one or more AAV ITRs flanking a nucleic acid molecule. AAV DNA in the rAAV genomes may be from any AAV serotype for which a recombinant virus can be derived including, but not limited to, AAV serotypes AAVrh.74, AAV-1, AAV-2, AAV-3, AAV-4, AAV-5, AAV-6, AAV-7, AAV-8, AAV-9, AAV-10, AAV-11, AAV-12 and AAV-13. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. To promote skeletal muscle-specific expression, AAV1, AAV6, AAV8 or AAVrh.74 may be used.

DNA plasmids of the invention comprise rAAV genomes of the invention. The DNA plasmids are transferred to cells permissible for infection with a helper virus of AAV (e.g., adenovirus, El-deleted adenovirus or herpesvirus) for assembly of the rAAV genome into infectious viral particles. Techniques to produce rAAV particles, in which an AAV genome to be packaged, rep and cap genes, and helper virus functions are provided to a cell, are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13. Production of pseudotyped rAAV is disclosed in, for example, WO 01/83692 which is incorporated by reference herein in its entirety.

Methods of generating a packaging cell comprise creating a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79: 2077-2081), addition of synthetic linkers containing restriction endonuclease cleavage sites (Laughlin et al., 1983, Gene, 23: 65-73) or by direct, blunt-end ligation (Senapathy & Carter, 1984, J. Biol. Chem., 259: 4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158: 97-129). Various approaches are described in Ratschin et al., Mol. Cell. Biol. 4: 2072 (1984); Hermonat et al., Proc. Natl. Acad. Sci. USA, 81: 6466 (1984); Tratschin et al., Mol. Cell. Biol. 5: 3251 (1985); McLaughlin et al., J. Virol., 62: 1963 (1988); and Lebkowski et al., Mol. Cell. Biol., 7: 349 (1988). Samulski et al., J. Virol., 63: 3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13: 1244-1250 (1995); Paul et al. Human Gene Therapy 4: 609-615 (1993); Clark et al. Gene Therapy 3: 1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

Also disclosed herein are packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with El of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the invention comprise a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the invention are set out in International Patent Application No. PCT/US2012/047999 (WO 2013/016352) incorporated by reference herein in its entirety.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the invention contemplates compositions comprising rAAV of the present invention. Compositions of the invention comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers and surfactants such as pluronics.

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1.times.10.sup.6, about 1.times.10.sup.7, about 1.times.10.sup.8, about 1.times.10.sup.9, about 1.times.10.sup.10, about 1.times.10.sup.11, about 1. times.10.sup.12, about 1.times.10.sup.13 to about 1.times.10.sup.14 or more DNase resistant particles (DRP) per ml. Dosages may also be expressed in units of viral genomes (vg).

In some aspects, an isolated nucleic acid as described herein comprises a region (e.g., a first region) comprising a first adeno-associated virus (AAV) inverted terminal repeat (ITR), or a variant thereof and a second region comprising a transgene encoding PGM1. The isolated nucleic acid (e.g., the recombinant AAV vector) can be packaged into a capsid protein and administered to a subject and/or delivered to a selected target cell. The transgene can also comprise a region encoding, for example, a protein and/or an expression control sequence (e.g., a poly-A tail).

Also disclosed herein are vectors comprising a single, cis-acting wild-type ITR. In some aspects, the ITR can be a 5′ ITR. In some aspects, the ITR can be a 3′ ITR. ITR sequences are about 145 bp in length. In some aspects, the entire sequences encoding the ITR(s) can be used in the molecule, although some degree of minor modification of these sequences is permissible. In some aspects, an ITR can be mutated at its terminal resolution site (TR), which inhibits replication at the vector terminus where the TR has been mutated and results in the formation of a self-complementary AAV. In some aspects, a “cis-acting” plasmid containing the transgene, in which the selected transgene sequence and associated regulatory elements can be flanked by the 5′ AAV ITR sequence and a 3′ hairpin-forming RNA sequence, can be used. AAV ITR sequences can be obtained from any known AAV, including presently identified mammalian AAV types. In some aspects, an ITR sequence can be an AAV2, AAV3, AAV4, AAV5, AAV6, AAV7, AAV8, AAVrh8, AAV9, AAV10, and/or AAVrh10 ITR sequence. In some aspects, the AAV ITR sequences can be AAV9.

Disclosed herein are pharmaceutical compositions comprising a therapeutically effective amount of any of the vectors disclosed herein, and a pharmaceutically acceptable carrier and/or adjuvant.

In some aspects, the composition (e.g., a pharmaceutical composition) can comprise an AAV or lentiviral vector comprising a nucleic acid encoding CNTNAP1.

In some aspects, one suitable carrier includes saline, which may be formulated with a variety of buffering solutions (e.g., phosphate buffered saline). Examples of other suitable carriers include but are not limited to sterile saline, lactose, sucrose, calcium phosphate, gelatin, dextran, agar, pectin, peanut oil, sesame oil, and water. Optionally, the compositions disclosed herein can also further include other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The formulation may be frozen until ready for use and then thawed and administered.

The compositions disclosed herein can be administered in sufficient amounts to transfect the cells of a desired tissue and to provide sufficient levels of gene transfer and expression without undue adverse effects. In some aspects, acceptable routes of administration include, but are not limited to, direct delivery to the selected organ (e.g., injection into the cerebral spinal fluid), oral, intravenous, intramuscular, subcutaneous, intradermal, intratumoral, and other parental routes of administration. In some aspects, the route of administration can be by direct injection. In some aspects, the route of administration can be by intravenous delivery. Routes of administration can be combined, if desired.

The dose of virions required to achieve a particular “therapeutic effect,” e.g., the units of dose in genome copies/per kilogram of body weight (GC/kg), the units of dose in genome copies per heart volume, will vary based on several factors including, but not limited to: the route of virion administration, the level of gene or RNA expression required to achieve a therapeutic effect, the specific disease or disorder being treated, and the stability of the gene or RNA product. One of skill in the art can readily determine a virion dose range to treat a patient having a particular disease or disorder based on the aforementioned factors, as well as other factors that are well known in the art.

An effective amount of the viral vector is an amount sufficient to target infect an animal, target a desired tissue. The effective amount will depend primarily on factors such as the species, age, weight, health of the subject, and the tissue to be targeted, and may thus vary among animal and tissue.

Formulation of pharmaceutically-acceptable excipients and carrier solutions are well-known to those of skill in the art, as is the development of suitable dosing and treatment regimens for using the particular compositions described herein in a variety of treatment regimens.

In some aspects, these formulations can contain at least about 0.10% of the active compound or more, although the percentage of the active ingredient(s) may, of course, be varied and can be conveniently be between about 1 or 2% and about 70% or 80% or more of the weight or volume of the total formulation. Naturally, the amount of active compound in each therapeutically-useful composition can be prepared in such a way that a suitable dosage can be obtained in any given unit dose of the compound. Factors such as solubility, bioavailability, biological half-life, route of administration, product shelf life, as well as other pharmacological considerations can be contemplated by one skilled in the art of preparing such pharmaceutical formulations, and as such, a variety of dosages and treatment regimens can be desirable.

In some aspects, it will be desirable to deliver the AAV-based therapeutic constructs in suitably formulated pharmaceutical compositions as disclosed herein either subcutaneously, intrapancreatically, intranasally, intracardiacally, parenterally, intravenously, intramuscularly, or orally, intraperitoneally, or by inhalation. In some aspects, the administration modalities as described in U.S. Pat. Nos. 5,543,158; 5,641,515 and 5,399,363 (each specifically incorporated herein by reference in its entirety) can be used to deliver AAVs. In some aspects, a preferred mode of administration can be intravenous delivery.

The pharmaceutical forms suitable for injectable use include sterile aqueous solutions or dispersions and sterile powders for the extemporaneous preparation of sterile injectable solutions or dispersions. Dispersions can also be prepared in glycerol, liquid polyethylene glycols, and mixtures thereof and in oils. Under ordinary conditions of storage and use, these preparations contain a preservative to prevent the growth of microorganisms. In many cases the form can be sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents, for example, sugars or sodium chloride can be included. Prolonged absorption of the injectable compositions can be brought about by the use in the compositions of agents delaying absorption, for example, aluminum monostearate and gelatin.

For administration of an injectable aqueous solution, for example, the solution can be suitably buffered, if necessary, and the liquid diluent first rendered isotonic with sufficient saline or glucose. These particular aqueous solutions can be suitable for intravenous, intramuscular, subcutaneous, and intraperitoneal administration. In this connection, a sterile aqueous medium that can be employed will be known to those of skill in the art. For example, one dosage can be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions can be prepared by incorporating the active AAV or lentivirus in the required amount in the appropriate solvent with various of the other ingredients enumerated herein, as required, followed by filtered sterilization. Generally, dispersions can be prepared by incorporating the various sterilized active ingredients into a sterile vehicle which contains the basic dispersion medium and the required other ingredients from those enumerated above. In the case of sterile powders for the preparation of sterile injectable solutions, the methods of preparation can be vacuum-drying and freeze-drying techniques which yield a powder of the active ingredient plus any additional desired ingredient from a previously sterile-filtered solution thereof.

The compositions disclosed herein can be also be formulated in a neutral or salt form. Pharmaceutically-acceptable salts, include the acid addition salts (formed with the free amino groups of the protein) and which can be formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations can be easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the AAV or lentiviral vector delivered transgenes can be formulated for delivery either encapsulated in a lipid particle, a liposome, a vesicle, a nanosphere, or a nanoparticle or the like.

Such formulations can be used for the introduction of pharmaceutically acceptable formulations of the nucleic acids constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes can be formed from phospholipids that can be dispersed in an aqueous medium and spontaneously form multilamellar concentric bilayer vesicles (also termed multilamellar vesicles (MLVs). MLVs generally have diameters of from 25 nm to 4 μm.

Sonication of MLVs results in the formation of small unilamellar vesicles (SUVs) with diameters in the range of 200 to 500 Angstroms, containing an aqueous solution in the core.

Alternatively, nanocapsule formulations of the AAV or lentivirus can be used. Nanocapsules can generally entrap substances in a stable and reproducible way. To avoid side effects due to intracellular polymeric overloading, such ultrafine particles (sized around 0.1 p.m.) should be designed using polymers able to be degraded in vivo. Biodegradable polyalkyl-cyanoacrylate nanoparticles that meet these requirements are contemplated for use.

In some aspects, the methods can include administering one or more additional therapeutic agents to a subject who has been administered any of pharmaceutical composition as described herein.

### Methods of Screening

Disclosed herein are transgenic mice and transgenic mouse lines, methods of producing a transgenic mouse and cell lines, and using the same. Disclosed herein are non-human transgenic animal models useful for screening drugs or candidate drugs and treating patient populations.

Disclosed herein are methods for screening a test substance for treating respiratory distress. In some aspects, the method can comprise: a) administering a test substance to any of the transgenic mice disclosed herein, and b) determining the effect of the test substance on respiratory distress. In some aspects, a decrease in the at least one symptom associated with respiratory distress as compared to a control indicates the test substance treats respiratory distress.

Disclosed herein are methods for screening a test substance for increasing nerve conduction. In some aspects, the methods can comprise: a) administering a test substance to any of the transgenic mice disclosed herein, and b) determining the effect of the test substance on nerve conduction. In some aspects, the effect on nerve conduction as compared to a control indicates the test substance increases nerve conduction.

Disclosed herein are methods for screening a test substance for reducing ataxia. In some aspects, the methods can comprise: a) administering a test substance to any of the transgenic mice disclosed herein, and b) determining the effect of the test substance on ataxia. In some aspects, the effect on ataxia as compared to a control indicates the test substance reduces ataxia.

Disclosed herein are methods for screening a test substance for reducing motor dysfunction. In some aspects, the methods can comprise: a) administering a test substance to any of the transgenic mice disclosed herein, and b) determining the effect of the test substance on motor dysfunction, wherein the effect on motor dysfunction as compared to a control indicates the test substance reduces motor dysfunction.

Disclosed herein are methods for screening a test substance for reducing or reversing paralysis. In some aspects, the methods can comprise: a) administering a test substance to any of the transgenic mice disclosed herein, and b) determining the effect of the test substance on paralysis. In some aspects, the effect on paralysis as compared to a control indicates the test substance reduces or reverses paralysis.

Disclosed herein are methods of screening for a biologically active agent for the treatment of neuropathies caused by CNTNAP1 mutations. Also, disclosed herein are methods for identifying biologically active agents or compounds (e.g., peptides, cyclic peptides, peptidomimetics, small molecules, small organic molecules, or other drugs) which effect (e.g., modulate, inhibit, reduce, prevent or reduce or increase) the outcome of neuropathies caused by CNTNAP1 mutations or one or more of the signs and symptoms of neuropathies caused by CNTNAP1 mutations. Agents or compounds identified as described herein can be used in an animal model to determine the mechanism of action, efficacy, toxicity or side effects of treatment with said agents or compounds.

Test compounds can be formulated into pharmaceutical compositions by admixture with pharmaceutically acceptable non-toxic excipients or carriers and administered to transgenic non-human animals described herein by any route of administration. For example, parenteral routes such as subcutaneous, intramuscular, intravascular, intradermal, intranasal, inhalation, intrathecal, or intraperitoneal administration, and enteral routes such as sublingual, oral, or rectal administration can be used.

Disclosed herein are methods of screening for a biologically active agent effective for the treatment of neuropathies caused by a CNTNAP1 mutation. In some aspects, the method comprises using the transgenic mouse or cell lines disclosed herein Also described herein, are in vitro screening methods. Disclosed herein are methods of screening for a biologically active agent effective for the treatment of neuropathies caused by a CNTNAP1 mutation. In some aspects, the cells can be contacted with the candidate agent for about between 72 to 96 hours. In some aspects, the cells can be contacted with the candidate agent for 1, 2, 3, 4, 5, 10, 15, 20, 25, 30, 40, 50, 60, 70, 80, 90, 100, 200 hours or any time period in between. Agents or compounds identified as described herein can be used in an animal model to determine the mechanism of action, efficacy, toxicity or side effects of treatment with said agents or compounds.

### Methods of Treating

Disclosed herein are methods of treating a subject or patient. In some aspects, the subject or patient is a human. In some aspects, the human subject is an infant or a child.

Disclosed herein are methods of treating and/or preventing Contactin-associated protein 1 (hCNTNAP1) protein deficiency in a subject in need thereof. In some aspects, the method can comprise administering to the subject the pharmaceutical composition comprising a therapeutically effective amount of any of the vectors of disclosed herein, and a pharmaceutically acceptable carrier and/or adjuvant.

Disclosed herein are methods of preventing or reducing severe respiratory distress in a subject in need thereof. In some aspects, the method can comprise administering to the subject the pharmaceutical composition comprising a therapeutically effective amount of any of the vectors of disclosed herein, and a pharmaceutically acceptable carrier and/or adjuvant.

Disclosed herein are methods of increasing nerve conduction in a subject in need thereof. In some aspects, the method can comprise administering to the subject the pharmaceutical composition comprising a therapeutically effective amount of any of the vectors of disclosed herein, and a pharmaceutically acceptable carrier and/or adjuvant.

Disclosed herein are methods of reducing ataxia in a subject in need thereof. In some aspects, the method can comprise administering to the subject the pharmaceutical composition comprising a therapeutically effective amount of any of the vectors of disclosed herein, and a pharmaceutically acceptable carrier and/or adjuvant.

Disclosed herein are methods of reducing motor dysfunction in a subject in need thereof. In some aspects, the method can comprise administering to the subject the pharmaceutical composition comprising a therapeutically effective amount of any of the vectors of disclosed herein, and a pharmaceutically acceptable carrier and/or adjuvant.

Disclosed herein are methods of reducing or reversing paralysis in a subject in need thereof. In some aspects, the method can comprise administering to the subject the pharmaceutical composition comprising a therapeutically effective amount of any of the vectors of disclosed herein, and a pharmaceutically acceptable carrier and/or adjuvant.

Disclosed herein are methods of ameliorating a symptom of a CNTNAP1 mutation in subject in need thereof. In some aspects, the method can comprise administering to the subject the pharmaceutical composition comprising a therapeutically effective amount of any of the vectors of disclosed herein, and a pharmaceutically acceptable carrier and/or adjuvant.

In some aspects, the symptoms of a CNTNAP1 mutation can be polyhydramnios, severe neonatal hypotonia, arthrogryposis, severe motor paralysis, acute respiratory distress, muscle atrophy or a combination thereof.

Disclosed herein are methods of treating a subject with a neuropathy caused by a CNTNAP1 mutation. In some aspects, the method can comprise administering to the subject the pharmaceutical composition comprising a therapeutically effective amount of any of the vectors of disclosed herein, and a pharmaceutically acceptable carrier and/or adjuvant.

Disclosed herein are methods of treating a patient with a neuropathy caused by CNTNAP1 mutations in need of treatment. In some aspects, the methods comprise administering to the patient a therapeutically effective amount of a lentivirus comprising a nucleic acid construct disclosed herein. In some aspects, the patient can be a human patient.

Any of the methods of detecting gene or protein expression can be used to identify a patient in need of treatment.

### EXAMPLES

### Example 1: Mouse Models of Human CNTNAP1 Mutations Cause Peripheral and Central Neuropathy

CNTNAP1 is an important constituent of axonal paranodal junctions in myelinated axons that connects the myelin sheath with the axon. The juxtaparanodal region is high in potassium channels (K+), the paranodal region contains CNTNAP1, and the nodal region is rich in sodium channels (Na+) while lacking a myelin sheath.

Disclosed herein are Cntnap1 mutant mice harboring the known human mutations generated using CRISPR-Cas9 technology that can be used to study the induced neuropathology. Compared to control mice, the mutant mice suffered mild/moderate weight loss from birth, and showed progressive neurological defects due to loss of nerve conduction in the peripheral nervous system.

The mutant CNTNAP1 protein was nearly undetectable from paranodal junctions and potentially disrupted the paranodal structure as revealed by western blot and transmission electron microscopy ultrastructural analysis. The paranodal junction proteins, Contactin and Neurofascinl55, were found to be mis-localized with a diffuse localization at the paranodal junctions by immunofluorescence analysis of sciatic nerve from mutant mice compared with age-matched healthy controls. In addition, such abnormal paranodal structure led to enlarged nodal region and potassium Kᵥ1.2 channel mislocalization in paranodes.

A transgenic mouse line was generated that expresses the inducible wild-type copy of CNTNAP1 protein upon Tamoxifen administration. The disclosed transgenic mouse line can be used to identity therapeutics that can be used to treat and/or rescue the phenotypes of the mutant mice. Additional expression of wild-type CNTNAP1 protein was found to be transported to the paranodal region in these mice, thereby restoring the functional axo-glial junctions. The rescue mice also showed significantly improved motor balance and coordination, faster nerve conduction velocities, and intact axo-glial junction structure.

The disclosed mouse lines are an important tool to study human CNTNAP1 gene mutations related to neurological disorder. For example, FIG. 20 shows the generation of Cntnap1 mouse mutant carrying human CNTNAPG1^{G3501/} mutation and their phenotypic analysis, and FIG. 21 shows that Cntnap^{G350/-} mutants display severe motor disability and decline in nerve conduction properties.

The therapeutic rescue approach can provide the wild-type Cntnap1 gene by either viral delivery or gene therapy, as these mutant proteins do not show dominant negative effects.

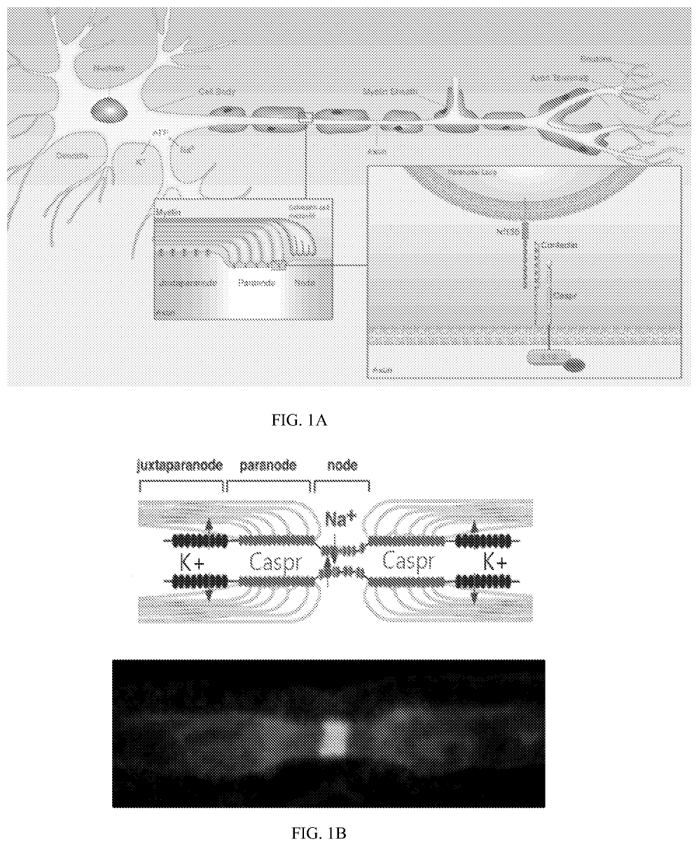

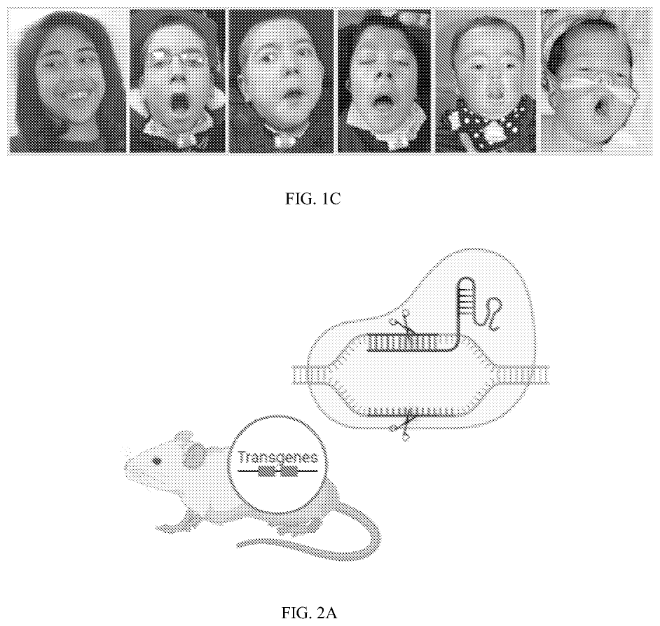

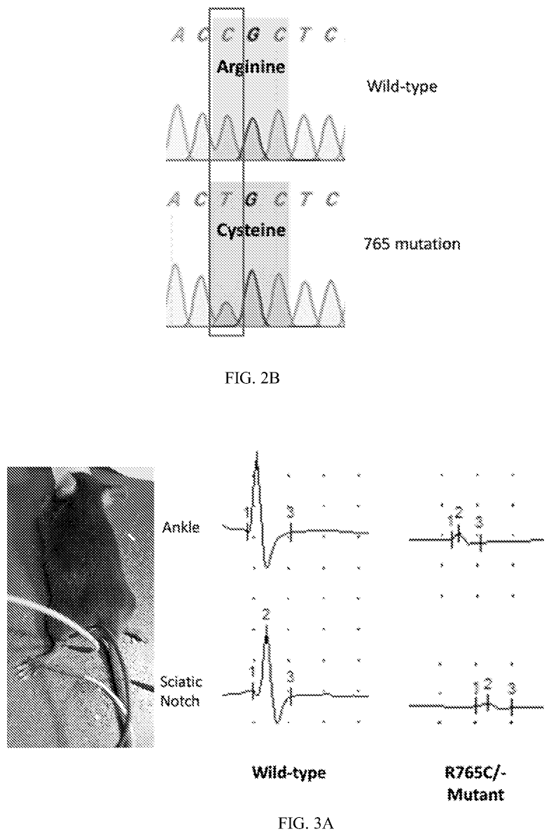

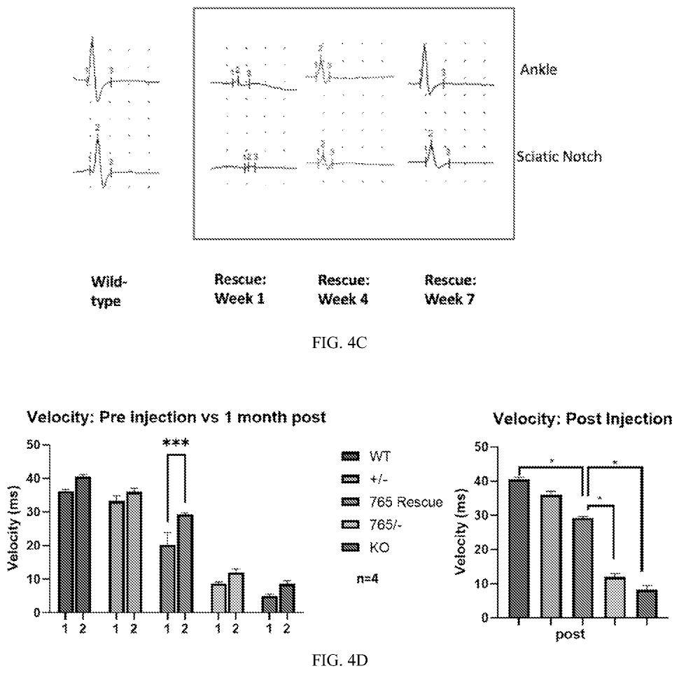

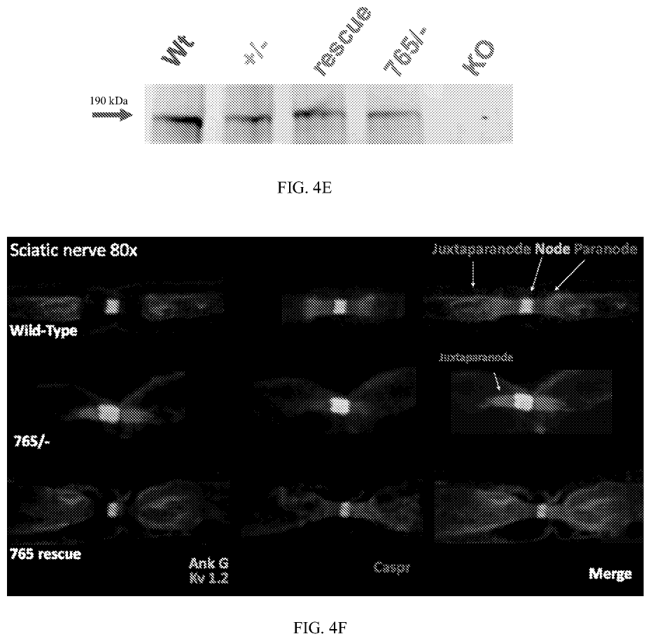

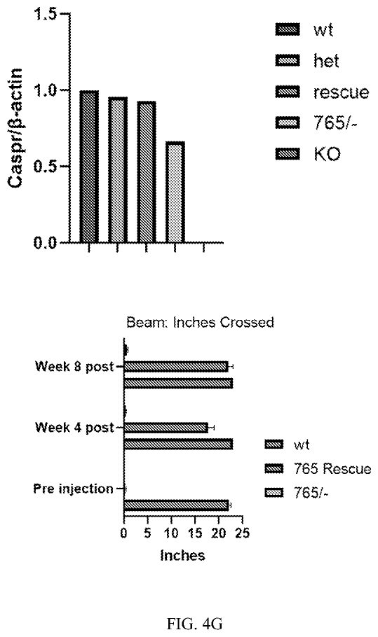

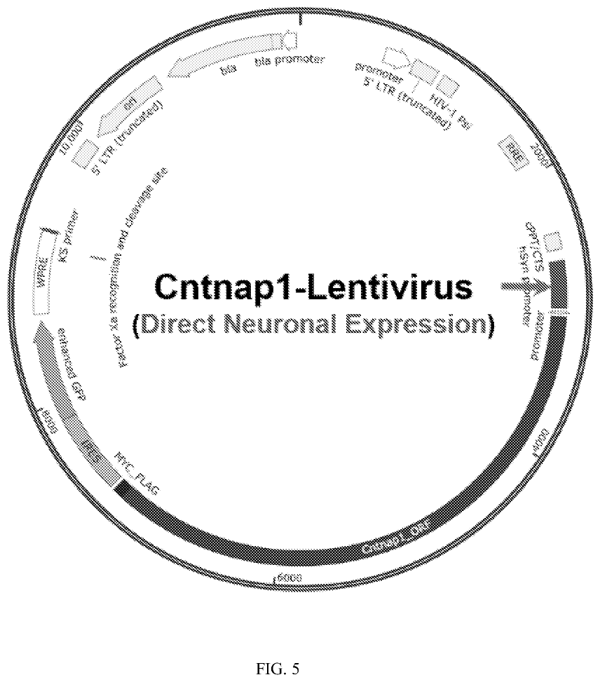

The transgenic mouse line generated and described herein can be referred to as LoxStopLox-CNTNAP1-Flag. As disclosed herein, the wild-type CNTNAP1 protein can be expressed in neurons upon Tamoxifen injection. The data shown in FIGS. 1-5 show that the overexpression of wild-type CNTNAP1 protein can rescue at least partially some of the mutant phenotypes, restore the functional axon-glial junctions and improve the motor balance and coordination. For example, see, the construct maps shown in FIGS. 18 and 19 used to generate the transgenic mice disclosed herein.

In sum, expression of exogenous Caspr rescues phenotype of R765C/− mutation, and led to the following observations: increases in nerve conduction amplitude and velocity, levels of Caspr expression increase in CNS and PNS, restoration of nodal domain disorganization, and increases in motor coordination.

### Example 2: Mouse Models of Human CNTNAP1-Associated Congenital Hypomyelinating Neuropathy and Genetic Restoration of Murine Neurological Deficits

The Contactin-associated protein 1 (Cntnap1) is an important component of the paranodes in myelinated axons and mice deficient in Cntnap1 fail to establish the paranodes. Mutations in human CNTNAP1 are linked to congenital hypomyelinating neuropathy-3, which causes neurological and motor deficits. To understand the neuropathology of these mutations, Cntnap1^{C324R} and Cntnap1^{R765C} mutant mice were generated to model human CNTNAP1 amino acid changes Cys323Arg and Arg764Cys, respectively. Both Cntnap1 mutant mice suffered weight loss, reduced nerve conduction, and showed progressive motor dysfunction. Mutant Cntnap1 proteins were nearly undetectable from the paranodes, and other paranodal proteins were also mislocalized. The paranodal ultrastructure showed everted myelin loops and absence of the axo-glial septate junctions. Both mutant proteins were primarily retained in the neuronal soma and showed reduced protein levels in the PNS and CNS compared to control Cntnap1. However, when the wild-type Cntnap1 gene was expressed postnatally in the Cntnap1 mutant backgrounds, the phenotypes improved, including increased nerve conduction, restoration of the paranodal domain, and organization of the axonal domains with improved motor function and coordination. The results described herein provide evidence of the mechanistic impact of two CNTNAP1 mutations in a mouse model and utility for gene therapy for CNTNAP1 patients.

As disclosed herein, the effects of two mutations, C324R and R765C, on the Cntnap1 protein were assessed. These mutations lead to misfolded proteins that are retained in the neuronal soma, likely in the endoplasmic reticulum. The normal Cntnap1 protein is mostly localized at the paranodal domains of myelinated axons. The retention of the mutant proteins affects the protein-protein interactions between Cntnap1 and Contactin, leading to reduced binding between the two proteins. Immunoprecipitation experiments were carried out to determine the effects of the mutations on protein-protein interactions. The results show that the binding between mutant CntnapC^{324R} and Cntnap1^{R765C} proteins and Contactin was severely affected, indicating that the mutations affected protein-protein interactions between Cntnap1 and Contactin. To investigate the effects of the mutations on protein expression, a Cntnap1 expression construct was used and independent constructs of Cntnap1^{C324R} and Cntnap1^{R765C} mutations were generated. The results show that the surface localization of both mutant proteins was strongly affected and that they were mostly retained in the cytoplasm, possibly in the endoplasmic reticulum. The surface expression of Contactin was also dramatically reduced in HEK cells co-transfected with either Cntnap1^{C324R} or Cntnap1^{R765C} constructs compared to cells co-transfected with the wild-type Cntnap1 protein.

The findings demonstrate that the C324R and R765C mutations in Cntnap1 have deleterious effects on the structure and function of the protein, as well as on the interactions between Cntnap1 and Contactin. The mutations lead to misfolded proteins that are retained in the neuronal soma and affect the formation of the axo-glial junctional complex between Cntnap1 and Contactin. Additionally, the mutations disrupt protein-protein interactions between Cntnap1 and Contactin and lead to defective co-transport and reduced cell surface expression of Cntnap1^{C324R} and Cntnap1^{R765C} mutant proteins.

Results. Human CNTNAP1 Mutations in Mice Severely Affect Postnatal Growth. Recent whole exome sequencing of children displaying generalized motor deficits with hypomyelination-associated neuropathologies has led to the identification of mutations in the CNTNAP1 gene. These mutations include single nucleotide changes in the CNTNAP1 coding sequence and reading frame shifts leading to terminations across the length of the CNTNAP1 protein (Laquerriere A, et al. Hum Mol Genet. 2014; 23(9):2279-2289; Sabbagh S, et al. Case Rep Med. 2020; 2020:8795607; and Vallat J M, et al. J Neuropathol Exp Neurol. 2016; 75(12):1155-1159). One of these mutations occurs at the coding nucleotide number 967, changing a TGC codon [Cysteine (Cys) 323] to CGC [Arginine (Arg) 323]. Another mutation occurs at the coding nucleotide number 2290, changing a CGC codon (Arg 764) to TGC (Cys 764). The Cys at 323 is located in the first Laminin G-like domain, and the Arg at is located in the Fibrinogen CT domain (FIG. 10A) (Vallat J M, et al. J Neuropathol Exp Neurol. 2016; 75(12):1155-1159; and (Vallat J M, et al. J Neuropathol Exp Neurol. 2016; 75(12):1155-1159; and Nizon M, et al. Eur J Hum Genet. 2017; 25(1):150-152). The amino acid sequence comparison between human CNTNAP1 and mouse Cntnap1 shows 93% identity and 96% similarity, and both Cys 323 and Arg 764 are highly conserved across many vertebrates whose sequences are also shown (FIG. 10B) (Bellen H J, et al. Trends Neurosci. 1998; 21(10):444-449).

To examine the consequences of human CNTNAP1 mutations and associated neuropathies in a genetically tractable mouse model system, CRISPR/Cas9 methodology was used to create single nucleotide changes that matched the codon substitution changes in human CNTNAP1 for Cys324Arg and Arg765Cys (note that Cys323 corresponds to Cys324 and Arg764 corresponds to Arg765 in mouse Cntnap1 and will be referred with mouse numbers herein). Upon pronuclear injections, the mouse progeny bom were first sequenced at the location of the mutations CGC and TGC codons encoding Arg324 and Cys765, respectively. The mouse lines that positively confirmed the presence of these nucleotide changes were further sequenced for the entire Cntnap1 gene to ensure that no additional mutations were introduced into the Cntnap1 coding sequences during the generation of these lines. Segments of sequences showing the homozygous wild-type Cntnap1 and Cntnap1^{R324} and Cntnap1^{C765} genotypes are shown in FIG. 10C. Thus, the sequence analyses confirmed that we have successfully generated the mouse models of the two human CNTNAP1^{R323} and CNTNAP1^{C764} mutations.

In human patients with CNTNAP1 mutations that cause Cys323Arg and Arg765Cys changes, the CNTNAP1 locus was found to be compound heterozygous, with a paternal stop-codon mutation and a maternal missense mutation (Laquerriere A, et al. Hum Mol Genet. 2014; 23(9):2279-2289; Vallat J M, et al. J Neuropathol Exp Neurol. 2016; 75(12):1155-1159; Mehta P, et al. Muscle Nerve. 2017; 55(5):761-765; Conant A, et al. J Child Neurol. 2018; 33(10):642-650; Nizon M, et al. Eur J Hum Genet. 2017; 25(1):150-152; Li W, et al. JCI Insight. 2020; 5(21); Letko A, et al. Genes (Basel). 2020; 11(12); and Lakhani S, et al. Eur J Med Genet. 2017; 60(5):245-249). Therefore, to mimic the human disease condition, Cntnap1^{C324R/+} and Cntnap1^{R765C/+} mice were crossed with Cntnap1^{+/-} (referred as +/−) mice, generated previously (Bhat M A, et al. Neuron. 2001; 30(2):369-383), to obtain the Cntnap1^{C324R/-} and Cntnap1^{R765C/-} (referred as C324R/− or R765C/−) mutants. The C324R/− or R765C/− mutant mice were bom at standard Mendelian ratio and were smaller than their heterozygous littermates. At postnatal day 15 (P15), the C324R/− or R765C/− mutant mice were photographed together with +/− and KO mutants (Bhat M A, et al. Neuron. 2001; 30(2):369-383), which showed overall body size reduction (FIG. 10D). Starting around P15, a significant reduction in motility, motor paresis, tremors and general muscle weakness was observed which were easily distinguishable from wild-type or heterozygous littermates. By P40, the weight difference between heterozygous control and Cntnap1 mutants was very notable, which showed that C324R/− or R765C/− were slightly better and displayed relatively higher body weights than the KO mutants (FIG. 10E) indicating that these single amino acid changes impact the functions of Cntnap1 and as a result the overall health of Cntnap1 mutant animals.

### Single Amino Acid Changes in Mouse Cntnap1 Affect its Stability and Cause

Paranodal Axonal Domain Disorganization. Cntnap1 is a transmembrane protein with a large extracellular N-terminal region and a short cytoplasmic C-terminal region. The single nucleotide substitutions in the Cntnap1 gene and the frameshift mutations may affect both the mRNA stability as well as protein expression levels (Li W, et al. JCI Insight. 2020; 5(21); and Sun X Y, et al. J Neuropathol Exp Neurol. 2009; 68(11):1207-1218). the total mRNA and protein levels of Cntnap1 in C324R/− and R765C/− mutants were examined. RT-PCR analysis detected no significant differences in Cntnap1 mRNA levels in the brains of both control +/− and C324R/− or R765C/− mutants. The immunoblotting assay using an antibody recognizing the C-terminal region of Cntnap1 (Bhat M A, et al. Neuron. 2001; 30(2):369-383), showed a robust single band of expected molecular weight of ˜190 kDa for the full-length Cntnap1 protein (FIG. 10F). While in the Cntnap1C324R/− and R765C/− mutants, the levels of the Cntnap1 protein were significantly reduced, the C324R/− mutants showed 64% and 88% reduction and the R765C/− showed 32% and 65% reduction in the CNS and PNS tissues, respectively, when compared to +/− controls (FIG. 10G). Thus, the immunoblot analysis shows that the stability of Cntnap1 is impacted by C324R and R765C amino acid substitutions.

Next, the expression of the C324R/− or R765C/− mutant proteins in the paranodal regions of the sciatic nerves (PNS, FIG. 1H-K) and the white matter of the spinal cord (CNS, FIGS. 10N-Q) were examined. In the PNS, the immunostaining of Cntnap1 revealed greatly reduced levels or barely detectable Cntnap1 at the paranodal regions detected by the anti-Cntnap1 antibody. However, there was variation in the staining patterns and the levels of Cntnap1 at the paranodes (FIGS. 10I, J). As the Cntnap1 staining in the paranodal region was significantly reduced or absent, it was next examined if this would affect the localization and distribution of juxtaparanodal K⁺ channels (Kᵥ1.2) and the voltage-gated nodal Na⁺ channels (Bhat M A, et al. Neuron. 2001; 30(2):369-383) (Bhat M A, et al. Neuron. 2001; 30(2):369-383; and Saifetiarova J, et al. J Neurosci. 2018; 38(28):6267-6282). In control +/− mice, both Na⁺ channels and K⁺ channels were separated at the distinct compartments by the paranodal region; in C324R/− (FIG. 10I) and R765C/− (FIG. 10J) mutants, Na+ channels were still restricted to the nodal regions but became widened; the juxtaparanodal K⁺ channels were completely mislocalized into the paranodal region and were in direct proximity to the nodal area, indicating a loss of paranodal function leading to the mislocalization of K⁺ channels in these Cntnap1 mutants in both the PNS (FIGS. 10H-K) and the CNS (FIGS. 10N-Q) (white arrows demarcate the boundaries of the juxtaparanodal regions). Thus, the immunostaining of the mutant myelinated fibers reveals a loss of the paranodal domain function in separating the juxtaparanodal proteins from the nodal complex.

To assess the expression levels of the Cntnap1 mutant proteins at the paranodes, the level of Cntnap1 was quantified across mutant paranodes by taking the strongest staining of Cntnap1 in +/− controls (as 100%). The relative intensities of the Cntnap1 levels were also quantified across mutant paranodes in the PNS (FIGS. 10H-K), which showed a very similar Cntnap1 staining pattern in C324R/− and R765C/− mutants with mean staining of about 20% of control and a significant number of paranodes with little or no Cntnap1 protein (FIGS. 10L, M). In the CNS, Cntnap1 staining was much weaker or nearly undetectable or absent in 90% of the paranodes in the white matter of C324R/− and R765C/− mutants compared to +/− controls (mean ˜10%) (FIGS. 10R, S). Together these data show that Cys324Arg and Arg765Cys amino acid substitutions affect the stability of Cntnap1 at the paranodes and also affect the barrier function of the paranodal domains in myelinated axons.

Cntnap1^{C324R} and Cntnap1^{R765C} Mutants Display Mislocalization of Important Paranodal Proteins but not their Protein Stability. Paranodal junctions in myelinated axons are highly specialized septate-like junctions. composed of three major proteins: Cntnap1 and Contactin (a GPI-anchored protein) on the axonal side (Bhat M A, et al. Neuron. 2001; 30(2):369-383; and Boyle M E, et al. Neuron. 2001; 30(2):385-397) and the 155 kDa glial Neurofascin (NF155) expressed on the myelin surface on the opposing side (Sherman D L, et al. Neuron. 2005; 48(5):737-742; and Pillai A M, et al. J Neurosci Res. 2009; 87(8):1773-1793). These three proteins are important for the organization of the paranodal domain axo-glial junctions, and their absence results in the disruption of the paranodal region and the axonal domain organization (Bhat M A, et al. Neuron. 2001; 30(2):369-383; Boyle M E, et al. Neuron. 2001; 30(2):385-397; Sherman D L, et al. Neuron. 2005; 48(5):737-742; and Pillai A M, et al. J Neurosci Res. 2009; 87(8):1773-1793). To determine whether the Cntnap1^{C324R} and Cntnap1^{R765C/-} mutant proteins affected the localization of the other paranodal components, the expression and localization of paranodal proteins in C324R/− and R765C/− mutants was examined. As shown in FIGS. 11A-F (PNS) and FIGS. 11M-R (CNS), the immunostaining showed the near absence of staining for both NF155 (FIGS. 11B, C; FIGS. 11N, O) and Contactin (FIGS. 11E, F; FIGS. 11Q, R) in the paranodal regions, similar to what has been observed for Cntanp1 null mutants (Bhat M A, et al. Neuron. 2001; 30(2):369-383). The localization of these paranodal proteins resembled the immunostaining pattern of C324R/− or R765C/− mutant proteins (refer to FIG. 10 for Cntnap1 expression). Quantitatively, the staining intensities of both NF155 and Contactin in the paranodal region were proportional to the staining levels of Cntnap1, suggesting that in the absence of proper functional Cntnap1, both NF155 and Contactin paranodal proteins were unable to localize properly at the paranodal regions [see quantification of fluorescence intensities for PNS (FIGS. 11G-L) and CNS (FIGS. 11S-X)]. Surprisingly, immunoblot analysis of the protein expression levels of these paranodal proteins NF155 and Contactin from the spinal cords of C324R/− and R765C/− mutants showed that the protein expression levels remained unchanged, both in the spinal cords (FIGS. 11Y, Z) and sciatic nerves. These data indicate that the C324R/− or R765C/− mutant proteins affect the paranodal localization of Contactin and NF155 but not their overall protein expression and stability in the tissues. Cntnap1^{C324R/-} and Cntnap1R765C-Mutants show Severe Motor Disability and a Significant Decline in the Peripheral Nerve Conduction Properties. To quantitatively measure the deficiency in fine motor coordination and balance, the beam walking trials, where the control and mutant mice were allowed to walk on a 50 cm long and 1 cm-diameter round-metal beams, was performed (schematic in FIG. 12A). Control (+/−), C324R/−, R765C/−, and KO mice were acclimated to the walking beam apparatus and then tested for their ability to walk across the beam. The C324R/−, R765C/−, and KO mice were unable to stand on the beam by themselves at the starting point of the beam walk test, whereas the control mice were able to stand steadily and cross the beam on the 1^{st} trial, suggesting a severe motor dysfunction in C324R/−, R765C/−, and KO mutant mice (FIG. 12B). Next, the Rotarod test which is used to assess fine motor coordination and steady balance on a rotation rod was performed. The control +/− mice stayed on the Rotarod for the entire experimental time (2 minutes with up to 25-35 rpm). In contrast, the C324R/−, R765C/−, and KO mutant mice started to fall off when the Rotarod started to acerate (FIGS. 12C, D). No significant differences were observed between C324R/−, R765C/−, and KO mutant mice indicating that the single amino acid and null mutants each showed severe fine motor coordination deficits.

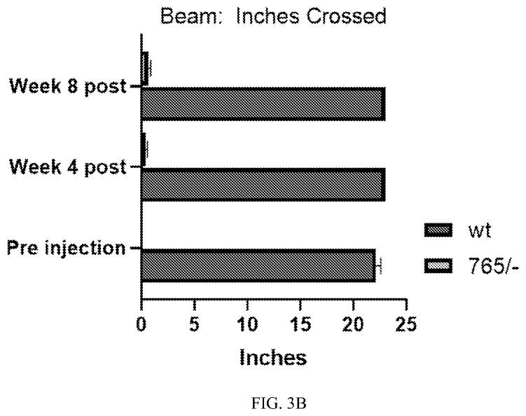

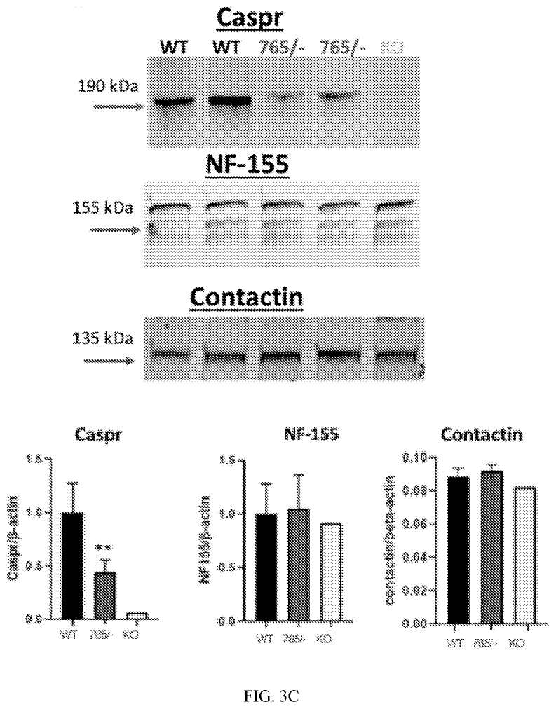

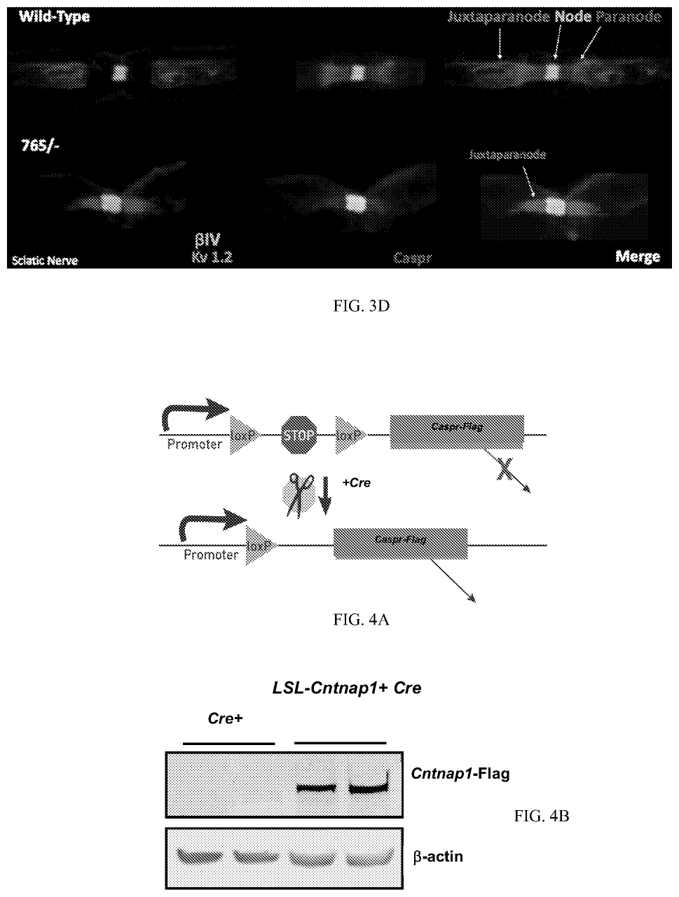

Next, the nerve conduction properties were analyzed by performing in-vivo recording of the sciatic nerves in control +/−, C324R/−, R765C/−, and KO mutant mice, littermates at P30. The representative traces of the nerve conduction measurements are presented for the sciatic nerve (FIG. 12E) and for the ankle region (FIG. 12I). The nerve conduction amplitudes for the sciatic nerve (FIG. 12F) and the ankle region (FIG. 12J) were significantly reduced in C324R/−, R765C/−, and KO mutant mice compared to control +/− mice. Similarly, the nerve conduction velocity (NCV) values were also significantly reduced in C324R/−, R765C/−, and KO mutant mice compared to control 1/− mice (sciatic nerve FIG. 3G) and (ankle FIG. 3K). There were no significant differences in amplitudes and NCV between C324R/−, R765C/−, and KO mutants. Meanwhile, the latency measuring the time taken for the fastest nerve fibers to conduct between two stimulation points, was significantly increased in C324R/−, R765C/−, and KO mutants (FIG. 12H and FIG. 12L). Interestingly, the heterozygous C324R+ and R765C/+ heterozygous mice did not display any abnormalities in any of the physiological parameters tested, indicating that in a heterozygous condition, the C324R and R765C mutations do not display any gain-of-function or dominant negative phenotypes.

Together, the in vivo nerve conduction electrophysiological measurements reveal a significant impact of Cntnap1 mutations on both the strength and velocity of the nerve impulses as is observed in KO mutants further highlighting the important role of Cntnap1 at the paranodal domain in myelinated axons.

Cntnap1^{C324R} and Cntnap1^{R765C} Mutants Display Hypomyelination and Loss of the Paranodal Axo-Glial Junctions. Upon the discovery of mutations in human CNTNAP1, it was observed that these mutations caused hypomyelination in both the central and peripheral nervous system, which has become the hallmark pathology for CNTNAP1-related congenital hypomyelinating neuropathies (CHNs, OMIM 605253) (Mehta P, et al. Muscle Nerve. 2017; 55(5):761-765; Conant A, et al. J Child Neurol. 2018; 33(10):642-650; Nizon M, et al. Eur J Hum Genet. 2017; 25(1):150-152; Wu R, et al. Neuropathology. 2019; 39(6):441-446; Lesmana H, et al. Pediatr Neurol. 2019; 93:43-49; Low K J, et al. Eur J Hum Genet. 2018; 26(6):796-807; and Hengel H, et al. Neurol Genet. 2017; 3(2):e144). In addition, delayed myelination of white matter was observed in the brain MRIs of human patients. As both C324R and R765C mutations are based on the human CNTNAP1 mutations identified in patients, a detailed electron microscopic (EM) analysis of the white matter axons was performed with respect to the extent of myelination and also how these mutations affected the ultrastructural organization of the paranodal region axo-glial junctions. EM analysis of myelinated axons from the spinal cords (CNS) and the sciatic nerves (PNS) from control +/−(FIGS. 13A & 13E), C324R/− (FIGS. 13B & 13F), R765C/−(FIGS. 13C & 13G), and KO (FIGS. 13D & 13H) mutants was carried out. While the visual inspection of the mutant myelinated fibers suggested hypomyelination in both the CNS and the PNS, a more quantitative analysis was performed that estimated the g ratios of the myelinated axons across the genotypes by a semi-automated software (Kaiser T, et al. eNeuro. 2021; 8(4)). G-ratio was significantly increased in C324R/− and R765C/− mutants and also in KO mutants when compared to control 1/− mice in both the CNS (FIG. 13I) and PNS (FIG. 13J). In the CNS, the average g-ratio in C324R/− mice was 33% higher than that in control +/− mice; while the average g-ratio in R765C/− mice was 21.6% higher than that in control mice. Interestingly, in the small axons (diameter smaller than 0.5 mm), the difference was even higher compared to control mice (FIGS. 13I, J). Similarly, the KO mutants also revealed hypomyelination and increased g-ratio in both the CNS and PNS as observed in C324R/− and R765C/− mutant myelinated axons evidencing that loss of Cntnap1 function results in hypomyelination.

The ultrastructure of the paranodal axo-glial junctions was examined to determine whether the C324R/− and R765C/− mutant proteins were able to establish axo-glial septate junctions, which are an important feature of the paranodal region. As shown in FIG. 13K (CNS) and FIG. 13N (PNS), the paranodal region in control +/− mice show distinct ladder-like septa (white arrowheads) between the myelin loops and the axonal axolemma in the paranodal area. In C324R/− and R765C/− mutants, the ladder-like paranodal axo-glial septate is absent in the CNS (FIGS. 13L, M) and PNS (FIGS. 13O, P) myelinated axons, respectively (white arrows). No septa-like structures were observed in C324R/− and R765C/− mutants similar to Cntnap1^{-/-} null mutants (Bhat et al., 2001). Taken together, the ultrastructural analysis of C324R/− and R765C/− mutant myelinated axons reveals hypomyelination and loss of the paranodal axo-glial septate junctions.

Neuronal Expression of the Wild-type Cntnap1 Causes Gradual Restoration of Axonal Domain Organization in Cntnap1^{C324R/-} and Cntnap1^{R765C/-} Myelinated Axons. Since Cntnap1 heterozygous mice that carried either a null allele or one mutant allele (C324R or R765C) did not display any noticeable abnormalities as compared to the +/+ littermates; it was determined whether timely controlled neuronal expression of the wild-type Cntnap1 gene in C324R/− and R765C/− mutants will be able to restore Cntnap1 expression as well as restore functional paranodal domains. To achieve that, a LoxP-Stop-LoxP-Cntnap1^{FLAG} (LSL-Cntnap1) knock-in mouse line (inserted into Rosa26 locus) was generated (Carofino B L, et al. Dis Model Mech. 2013; 6(6):1494-1506). The presence of the upstream multi-polyadenylation [poly(A)] sequences terminate transcription and prevent the expression of the downstream Cntnap1^{FLAG} cDNA (FIG. 14A). Upon expression of tissue-specific Cre recombinase, sequences between the LoxP sites are removed allowing transcription of the Cntnap1^{FLAG} cDNA and expression of Cntnap1^{Flag} only in selected Cre-expressing cells (FIG. 14A). To confirm the expression of the Cntnap1 protein in LSL-Cntnap1 mice, this line was crossed with the ubiquitous f-Actin-Cre line and examined the expression of the Cntnap1^{Flag} protein. As shown in FIG. 14B, immunoblotting of the spinal cord lysates with an anti-Flag antibody detected the Cntnap1^{Flag} protein at ˜190 kDa only in Actin-Cre;LSL-Cntnap1^{Flag}. The protein was undetectable in the LSL-Cntnap1^{Flag} spinal cord lysates which did not express the Cre recombinase indicating that there is no leaky expression in the LSL-Cntnap1^{Flag} mouse line (FIG. 14B). Immunostaining of the sciatic nerve myelinated axons using a combination of anti-Flag and anti-Cntnap1 antibodies (Bhat M A, et al. Neuron. 2001; 30(2):369-383) showed that the Cntnap1^{Flag} protein (FIG. 14Cd, green) was properly localized at the paranodes as it colocalized with the endogenous Cntnap1 (FIG. 14Ce, red, and in merged image FIG. 14Cf). Without the Cre-recombinase expression, the Cntnap1^{Flag} protein was undetectable in the paranodal domain in LSL-Cntnap1^{Flag} myelinated axons (FIG. 14Ca, green) which were positively labeled by anti-Cntnap1 antibodies (FIG. 14Cb, red, and merged image FIG. 14Cc). Together, these data show that transgenic Cntnap1^{Flag} protein expression is tightly controlled with no leakiness and when expressed in a Cre-dependent manner is able to localize at the paranodal region in myelinated axons.

Next, it was tested whether induced expression of Cntnap1 in C324R/− and R765C/− mutant background will allow restoration of the paranodal domain as well as re-localization of the juxtaparanodal proteins back to the juxtaparanodes. LSL-Cntnap1 mice were crossed with the tamoxifen-inducible SLICK-H-CreERT2 mice which express the Cre-recombinase in the neuron (Saifetiarova J, et al. J Neurosci. 2018; 38(28):6267-6282; Heimer-McGinn V, and Young P. Genesis. 2011; 49(12):942-949; and Taylor A M, et al. eNeuro. 2018; 5(3)). The final mice of the genotype SLICK-H-CreER; LSL-Cntnap1^{Flag}; C324R/− (referred here as TgEx;C324R/−) or SLICK-H-CreER;LSL-Cntnap1^{Flag};R765C/− (referred here as TgEx;R765C/−) received tamoxifen at P21 for 5 consecutive days (at 1 mg/kg body weight). The control animals LSL-Cntnap1^{Flag}; C324R/− (C324R/−) and LSL-Cntnap1^{Flag};R765C/− (R765C/−) without Cre also received the same regimen of tamoxifen. The animals were sacrificed 2 weeks and 7 weeks post-tamoxifen injection and processed for immunoblot and immunostaining analysis. As shown in FIG. 14D, transgenic Cntnap1 expression showed a steady increase from 2 weeks to 7 weeks in TgEx;C324R/− mice compared to C324R/− mice. The +/− and KO mice were also analyzed for expression for comparison. The Cntnap1 protein levels were quantified (FIG. 14E) which showed a significant increase in Cntnap1 levels at 7 weeks post-tamoxifen treatment.

Immunostaining of the sciatic nerves (FIGS. 14F-H) and spinal cords (FIGS. 14I-K) from control C324R/− and TgEx;C324R/− using triple antibodies against Cntnap1^{Flag} (red), juxtaparanodal Kᵥ1.2 (green) and nodal PIV Spectrin (blue) showed expression of Cntnap1^{Flag} at the paranodes in 2-week post-tamoxifen injection and most notably much more prominently in the 7 week-post-tamoxifen fibers (FIG. 5H, red). In both the PNS (FIGS. 14F-H) and CNS (FIGS. 14I-K), at 2 weeks the juxtaparanodal Kᵥ1.2 channels (green) were still observed in the paranodal area, but by 7 weeks post-injection most of the Kᵥ1.2 channels were separated from the paranodal region (FIGS. 14H, K). In control C324R/− fibers, the juxtaparanodal Kᵥ1.2 (green) channels are present in the paranodal area adjacent to nodal PIV Spectrin (blue). The relative expression of Cntnap1^{Flag} at the paranodes was quantified in the relevant genotypes as shown in FIG. 14L. The reorganization of the paranodes and separation of the juxtaparanodal Kᵥ1.2 from the paranodal area were also quantified which showed that in 7 weeks after expression of Cntnap1^{flag}, the paranodal domain formation is indistinguishable from the control heterozygous myelinated fibers (FIG. 14M). Similarly, immunoblotting of spinal cords from 2 weeks to 7 weeks after tamoxifen injections in TgEx;R765C/− mice was compared to R765C/−. The +/− and KO mice were also analyzed for Cntnap1 expression comparison (FIG. 14N). The Cntnap1 protein levels were quantified as shown in FIG. 14O, which showed a significant increase in Cntnap1 levels at 7 weeks post-tamoxifen treatment in TgEx;R765C/− mice. Immunostaining of the sciatic nerves (FIGS. 14P-R) and spinal cords (FIGS. 14S-U) from R765C/− mutant and TgEx;R765C/− using triple antibodies against Cntnap1^{Flag} (red), juxtaparanodal Kᵥ1.2 (green) and nodal PIV Spectrin (blue) also showed expression of Cntnap1^{Flag} at the paranodes in 2-week post-tamoxifen injection fibers and most notably much more prominently in the 7 week-post-tamoxifen fibers (FIGS. 14R, U). In both the PNS (FIGS. 14P-R) and CNS (FIGS. 14S-U), at 2 weeks, the juxtaparanodal Kᵥ1.2 channels (green) were observed in the paranodal area as in TgEx;C324R/− fibers; but by 7 weeks post-injection, most of the Kᵥ1.2 channels were clearly separated from the paranodal region. Similar in the case of R765C/− mutant, the relative expression of Cntnap1^{Flag} at the paranodes in TgEx;R765C/− fibers was quantified (FIG. 14V), and the reorganization of the paranodes and separation of the juxtaparanodal Kᵥ1.2 from the paranodal area was also quantified (FIG. 14W), which showed that in 7-weeks after expression of Cntnap1^{flag}, the paranodal domain formation is indistinguishable from the control heterozygous myelinated fibers. Additional immunostaining using antibodies against two other well-characterized paranodal proteins (NF155 and Contactin) also revealed their restoration at the paranodes and proper organization of the axonal domains in C324R/− and R765C/− mutants upon Cntnap1^{Flag} induction. These data demonstrate that the transgenic Cntnap1^{Flag} protein is able to rescue and restore the paranodal domain and accordingly the organization of the axonal domain in mutant myelinated axons. These data further demonstrate that Cntnap1^{C324R} and Cntnap1^{R765C} mutations are recessive and do not act as dominant negative or cause any gain of function phenotypes in C324R/− and R765C/− mutants.

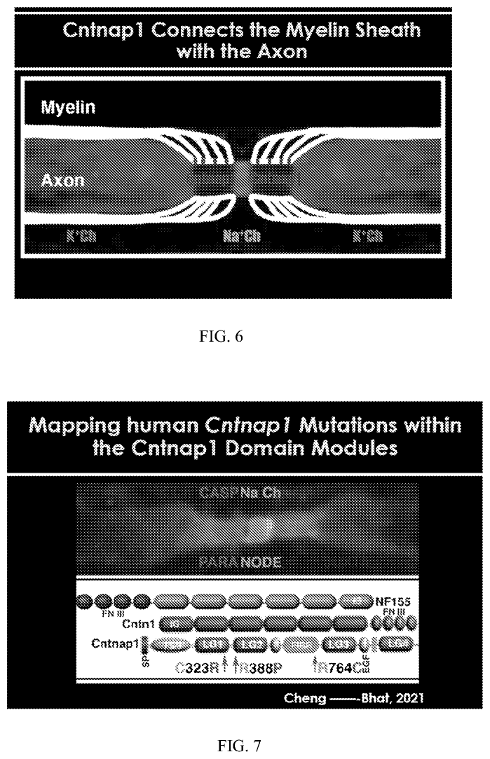

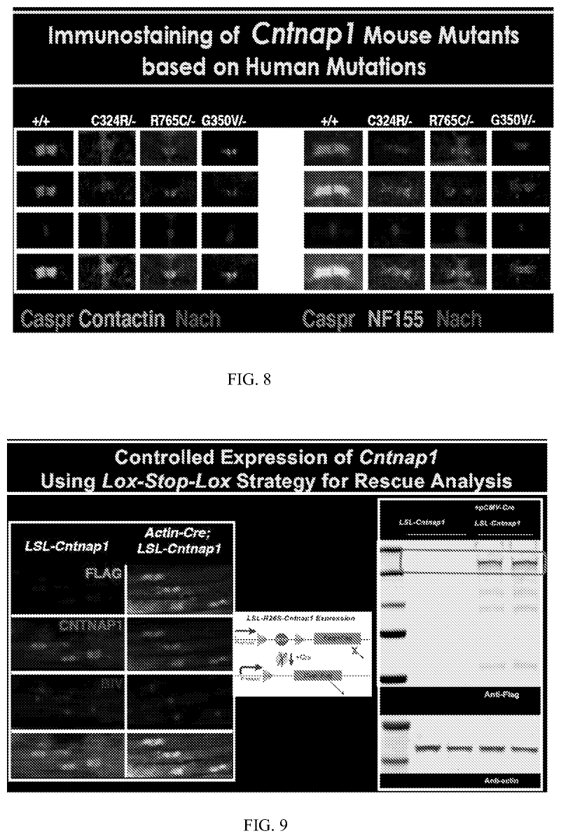

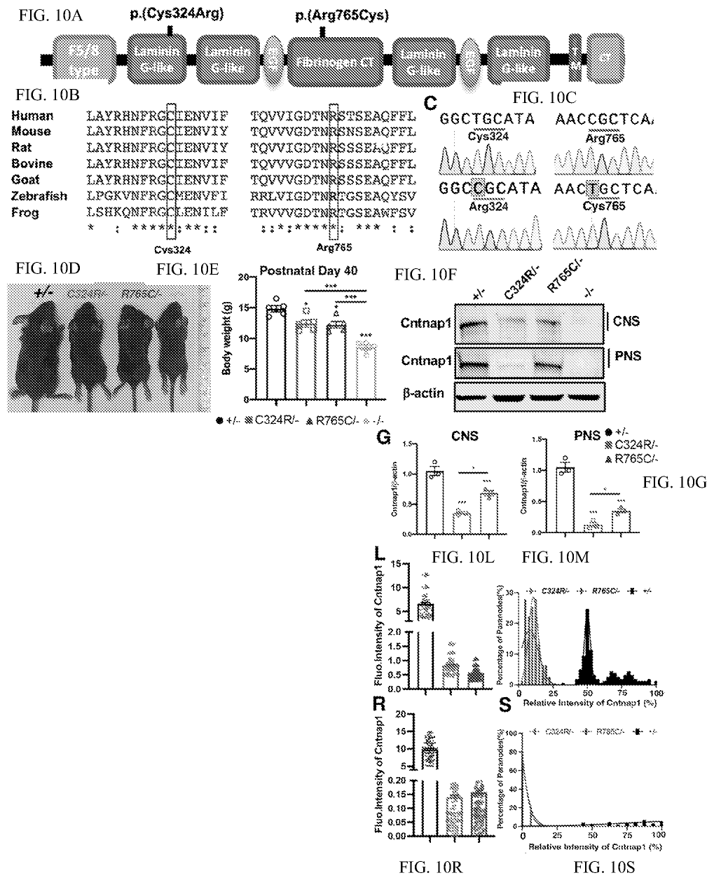

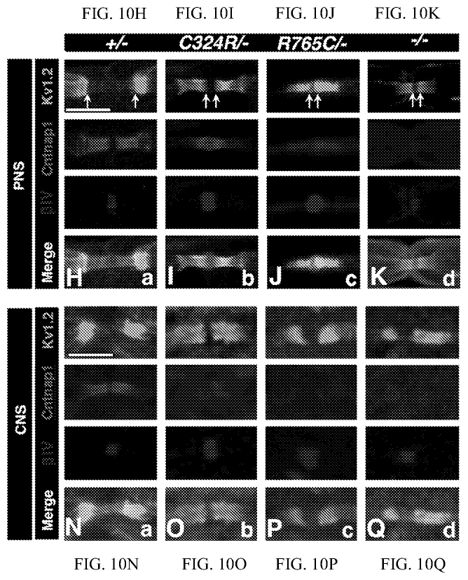

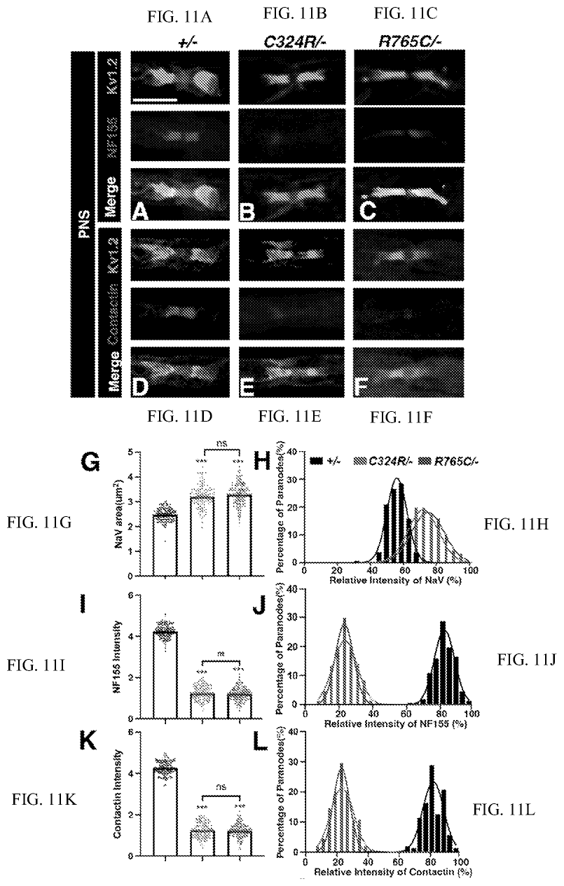

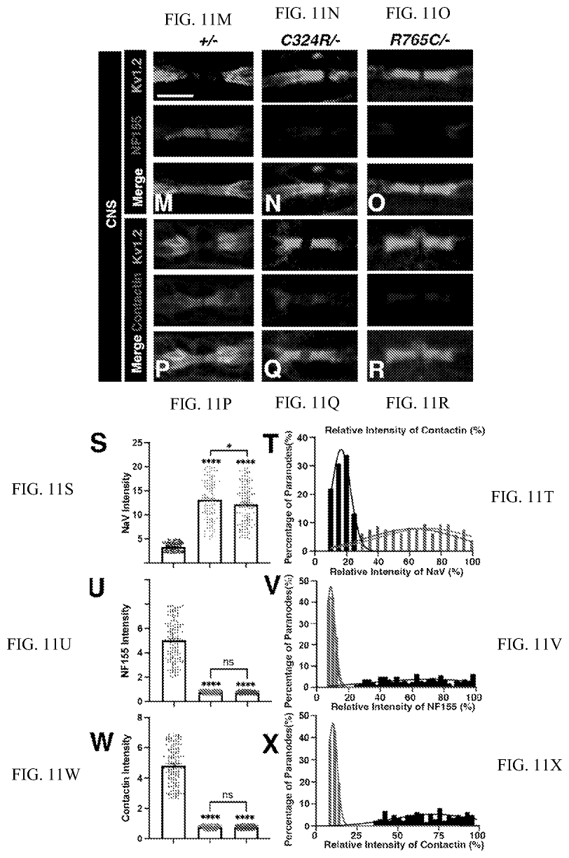

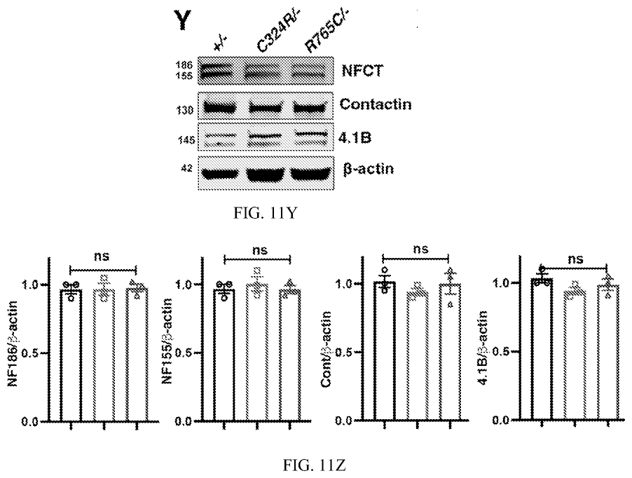

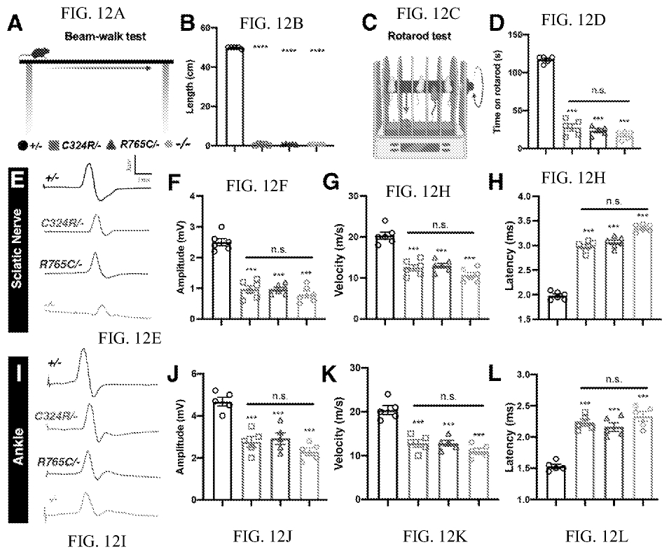

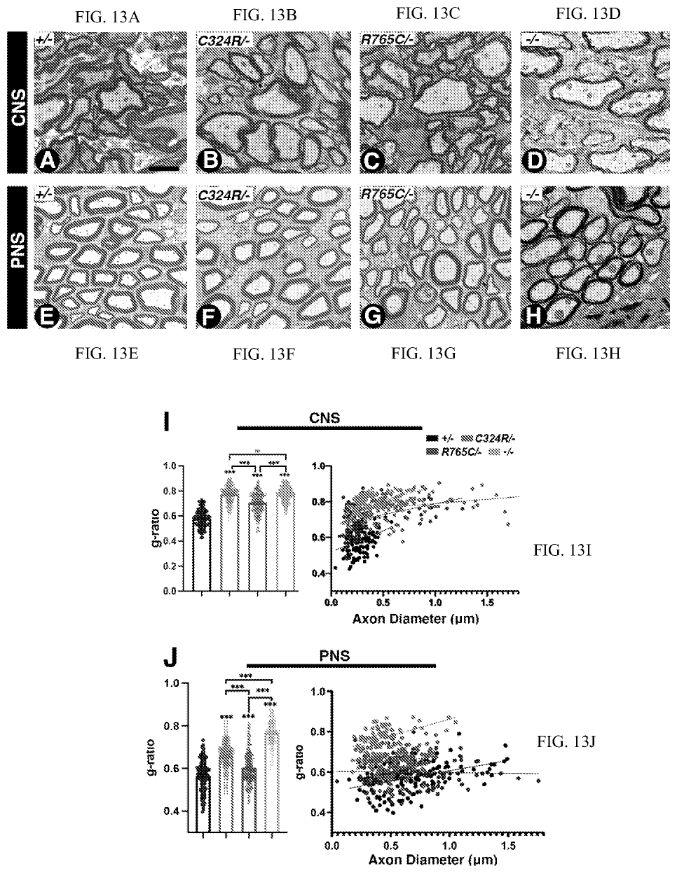

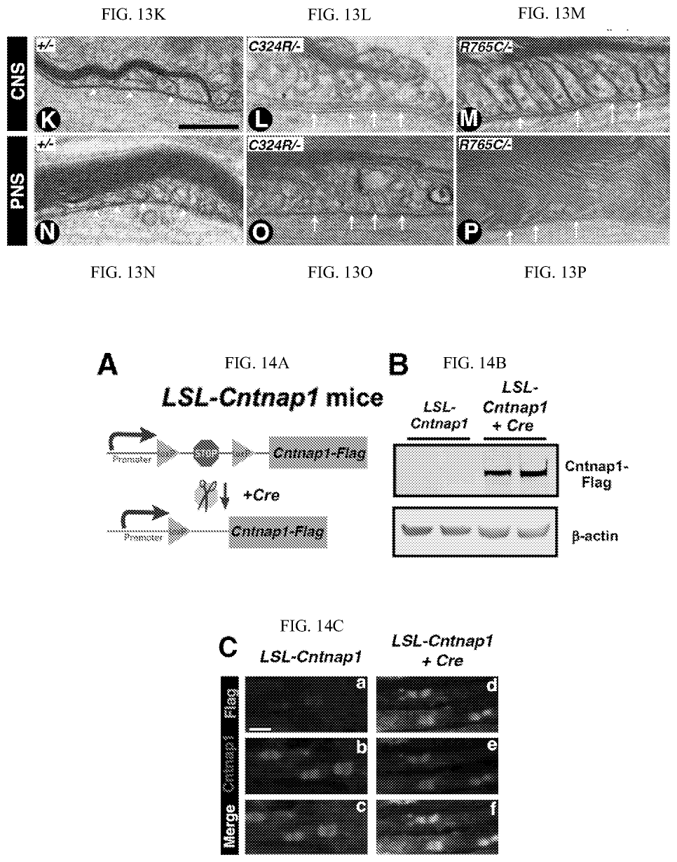

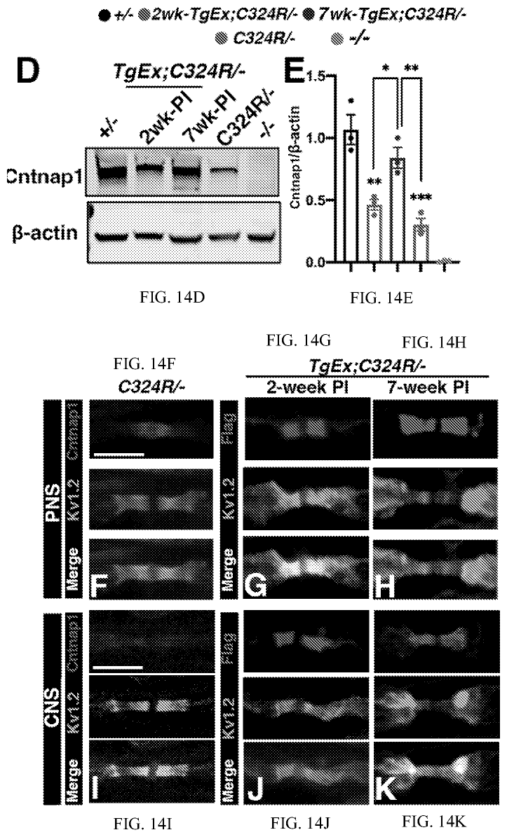

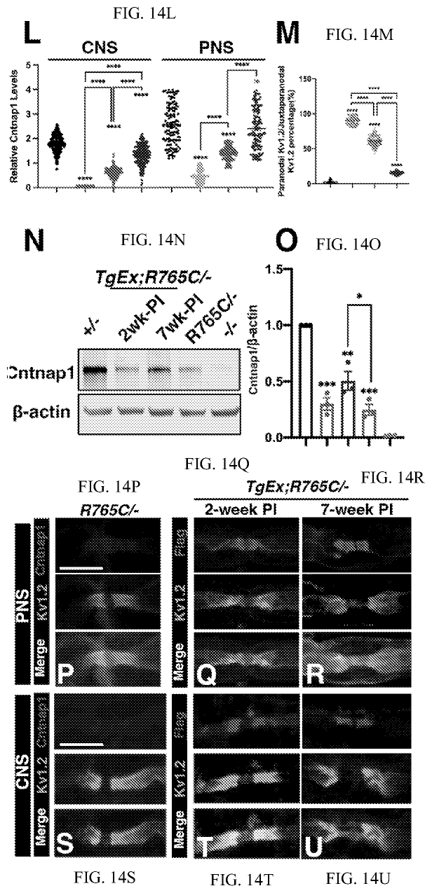

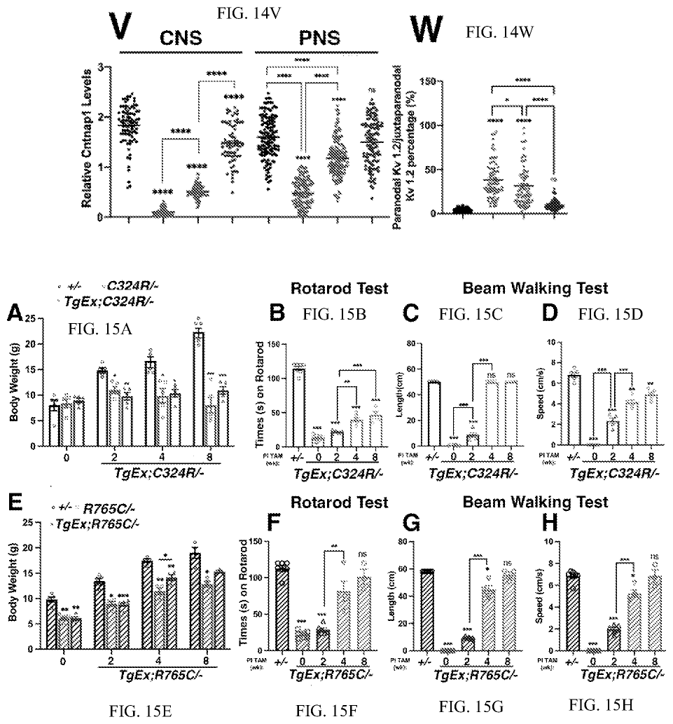

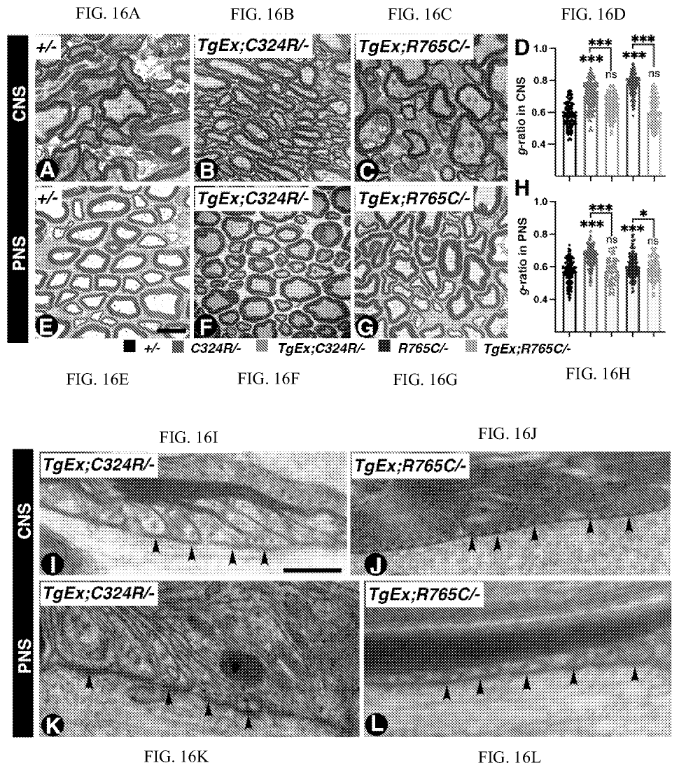

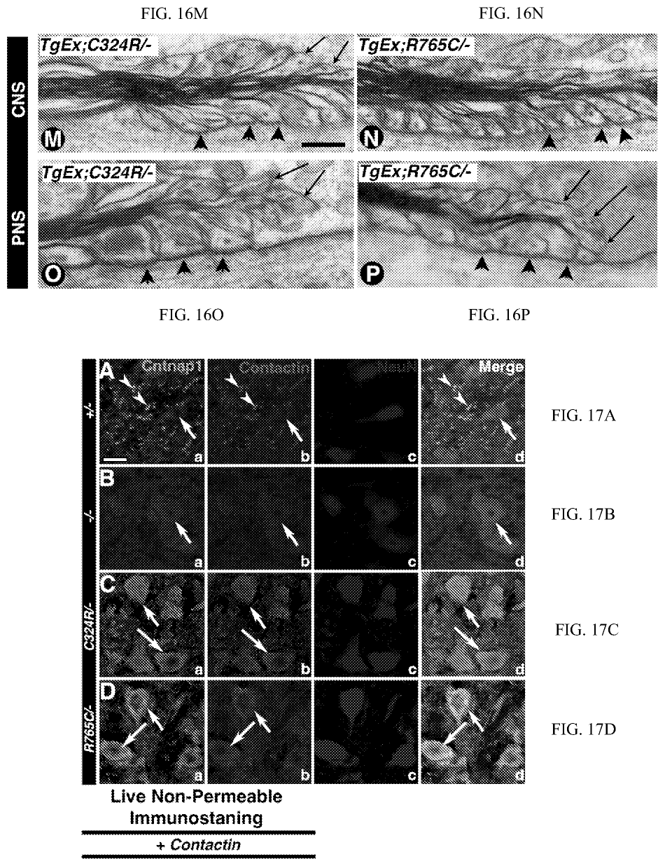

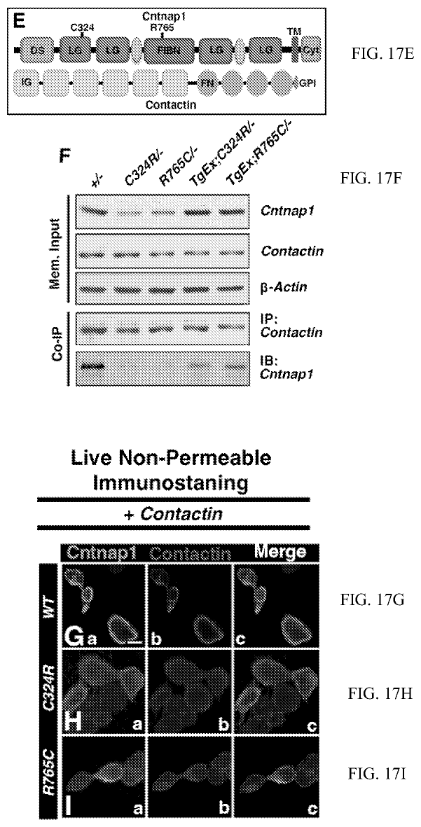

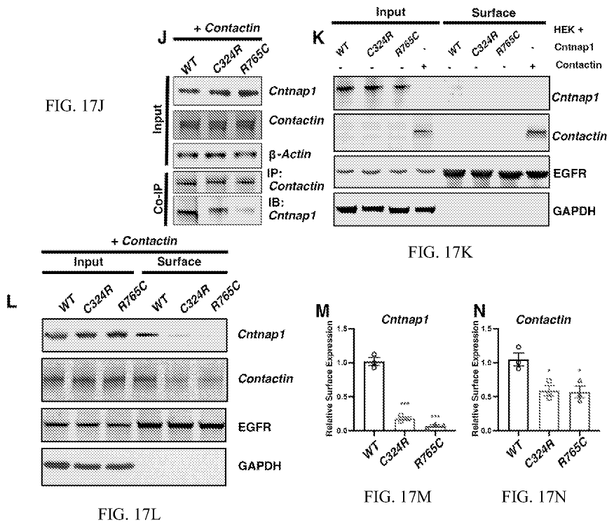

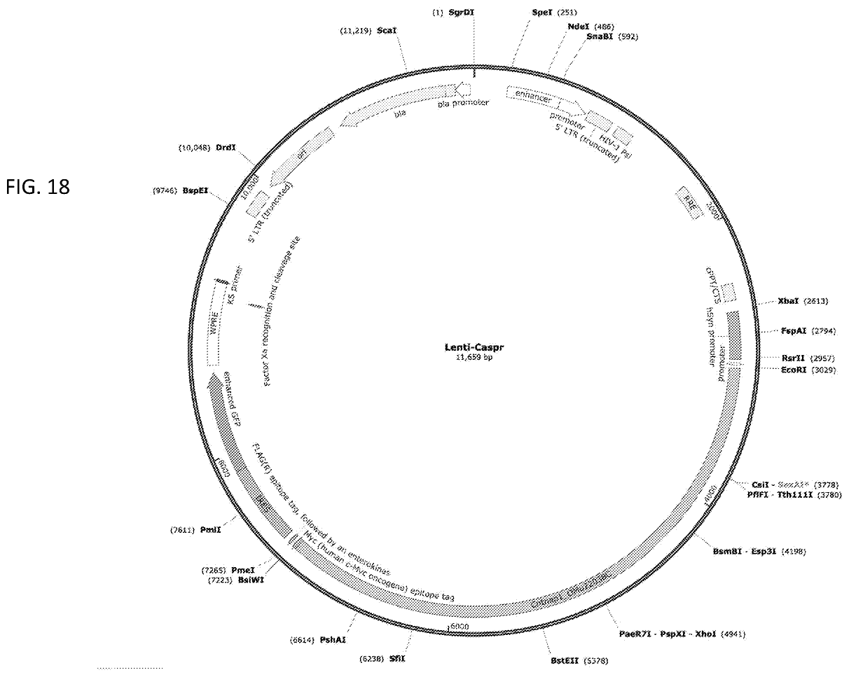

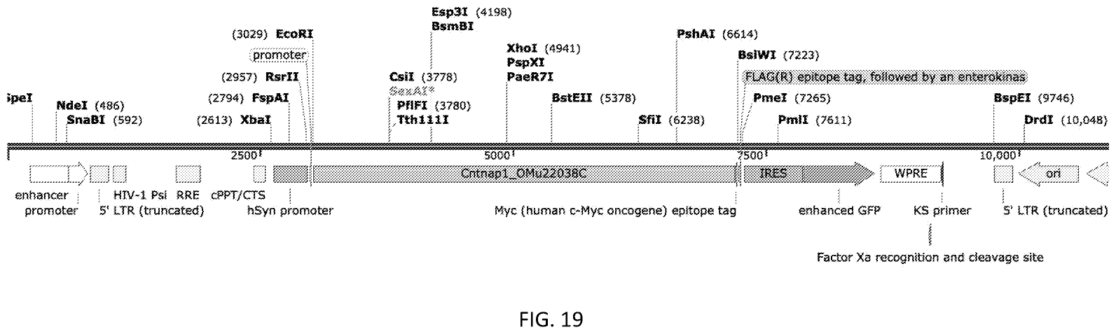

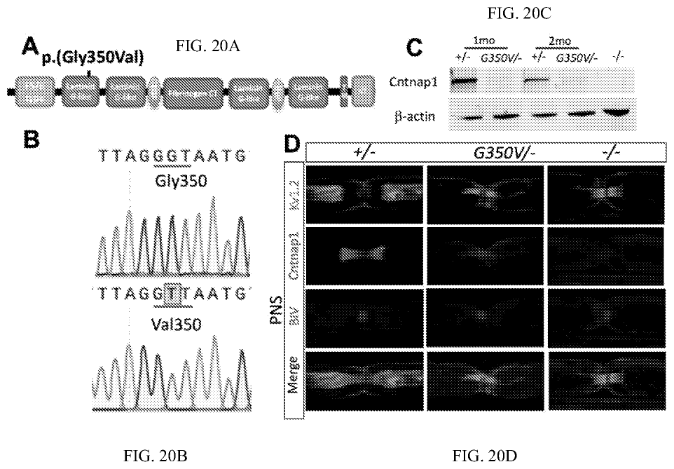

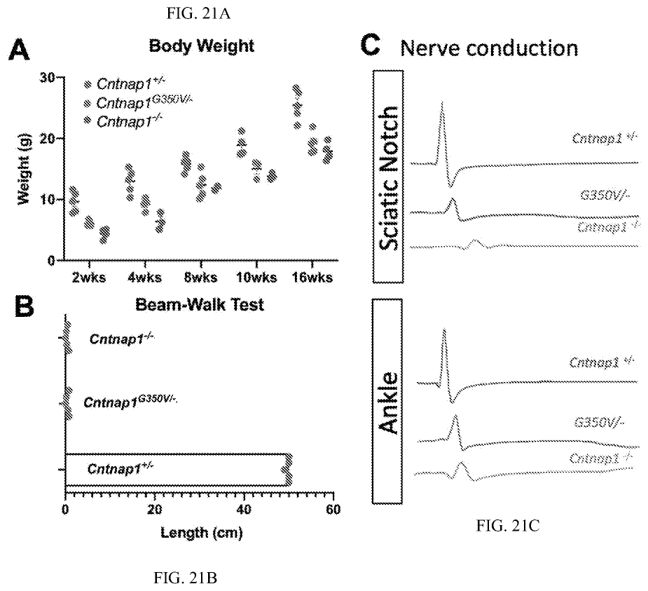

Neuronal Expression of the Wild-type Cntnap1 in Cntnap1^{C324R/-} and Cntnap1^{R765C/-} Mutants Restores Motor Coordination. Since it was observed that robust expression of the induced Cntnap1^{Flag} protein in the paranodal region and restoration of axonal domains in myelinated axons, it was next determined whether TgEx; C324R/− and TgEx;R765C/− mice will also show improvements in the body weight gain and motor coordination after tamoxifen injection for extended periods. The body weight and motor coordination using beam walking and Rotarod were recorded at 2, 4, and 8 weeks after tamoxifen injection. As shown in FIGS. 15A, E, the body weight gains were modest and did not show dramatic gains even after 8 weeks; however, these gains were better than control C324R/− and R765C/− mutants. The body weight gain may have to do with the timing of tamoxifen injection (3 weeks after birth) and by then the mutant animals may have already undergone some muscle atrophy. For motor coordination using the Rotarod test, the TgEx;C324R/− and TgEx;R765C/− mutant mice started to perform better from 8 weeks post tamoxifen injection, and the motor performance was only slightly enhanced in 10 weeks post-injection. In comparison, TgEx;C324R/− and TgEx;R765C/− mutant mice showed a significant difference when compared to uninduced littermates (FIGS. 15B, F). For beam walking motor performance, within 2 weeks after tamoxifen injection, it was observed a significant improvement. The TgEx;C324R/− and TgEx;R765C/− mutant mice were able to walk a short distance on the beam, which improved progressively such that the rescued TgEx; C324R/− and TgEx;R765C/− mutants completed the entire beam distance (50 cm) like Cntnap1^{+/-} control animals (FIGS. 15C, G). The speed on the beam walking also improved progressively and was close to 80% for rescued TgEx;C324R/− mutants or close to 100% for TgEx;R765C/− rescued mutants compared to control animals (FIGS. 6D, H). Together, these data demonstrate that the wild-type Cntnap1 is able to progressively restore motor coordination and function in Cntnap1^{C324R} d Cntnap1^{R765C} mutants.

Expression of Cntnap1 Rescues Hypomyelination and Restores the Ultrastructure of Paranodal Axo-Ghial Junctions in Cntnap1^{C324R/-} and Cntnap1^{R765C/-} Mutants. Human subjects carrying CNTNAP1 mutations have shown hypomyelination phenotypes indicative of the role of CNTNAP1 in axonal myelination (Conant A, et al. J Child Neurol. 2018; 33(10):642-650; and Hengel H, et al. Neurol Genet. 2017; 3(2):e144). In addition, both C324R/− and R765C/− mutants revealed hypomyelination and reduced g-ratios in myelinated axons (FIG. 13). Next, it was determined whether the hypomyelination phenotype in C324R/− and R765C/− mutants could be rescued by neuronal expression of Cntnap1 in both the CNS and PNS myelinated axons. Spinal cords (CNS) (FIG. 7A-C) and sciatic nerves (PNS) (FIGS. 16E-G) were processed from +/−, TgEx; C324R/− and TgEx; R765C/− animals which received tamoxifen at P21 for 5 consecutive days and were analyzed at 8 weeks post-tamoxifen injections. Quantification of the g-ratios for the CNS (FIG. 16D) and PNS (FIG. 16H) myelinated axons from +/−, TgEx; C324R/− and TgEx; R765C/− animals revealed that the hypomyelination phenotypes in the Cntnap1 mutants was significantly rescued (FIGS. 16D, H) by neuronal expression of Cntnap1 indicating that Cntnap1 is required for proper axonal myelination. The longitudinal sections of the myelinated axons were analyzed from TgEx; C324R/− and TgEx; R765C/− animals to determine whether Cntnap1 expression restored the ultrastructural organization of the paranodal axo-glial junctions given that Cntnap1 expression allowed the restoration of the paranodal domain in C324R/− and R765C/− mutants (FIG. 14). High-resolution EM images from the spinal cord (CNS) fibers of TgEx; C324R/− (FIG. 16I) and TgEx; R765C/− (FIG. 16J) and from sciatic nerves (PNS) of TgEx; C324R/− (FIG. 16K) and TgEx; R765C/− (FIG. 16L) showed restoration of the paranodal axo-glial junctions (FIGS. 161-L, black arrowheads). Most notably, the stereotypical septate-like junctions were clearly visible in the rescued animal fibers as observed in control (Cntnap1^{+/-}) myelinated fibers and absent in C324R/− and R765C/− mutants (see, FIG. 13). However, the inverted myelin loops presented in both TgEx; C324R/− (FIGS. 16M, O) and TgEx; R765C/− (FIGS. 16N, P) mice were noticeable, suggesting the restoration of paranodal junction would not fully revert the myelin loop pathology. Together, these data demonstrate that neuronal expression of Cntnap1 is able to restore myelination and paranodal axo-glial junction formation in C324R/− and R765C/− mutants.

C324R/− and R765C/− Mutant Proteins Remain in the Neuronal Soma and Reveal Reduced Binding with Contactin. Many amino acid changes in proteins occurring due to mutations in their encoding genes lead to conformational changes in protein structure resulting in altered and potentially misfolded proteins. Mutant proteins within the cell either get retained in the endoplasmic reticulum (ER) (Volpi V G, et al. Front Mol Neurosci. 2016; 9:162; Ron D, and Walter P. Nat Rev Mol Cell Biol. 2007; 8(7):519-529; and Wang S, and Kaufman R J. J Cell Biol. 2012; 197(7):857-867) or get further processed through autophagy and degraded through the ubiquitin/proteasome pathway (Devanathan V, et al. J Neurosci. 2010; 30(27):9292-9305). In both Cntnap1^{C324R} and Cntnap1^{R765C} mutant proteins, potential alternations in disulfide bonds or conformational changes are predicted to significantly change the native state of the Cntnap1 protein. Immunostaining of spinal cords from control +/−, KO, C324R/− and R765C/− mutants using antibodies against Cntnap1 (FIGS. 17Aa-Da, green), Contactin (FIGS. 17Ab-Db, red), and NeuN (FIGS. 17Ac-Dc, blue) and in merged images (FIGS. 17Ad-Dd) revealed that both Cntnap1^{C324R} (FIGS. 17Ca, d) and Cntnap1^{R761C} (FIGS. 817 Da, d) mutant proteins were present in the neuronal soma (white arrowheads), which was not observed in the control +/− neurons (FIGS. 17Aa, d).

Interestingly, the Contactin protein was also retained in the neuronal somas of C324R/− and R765C/− mutants (white arrows), which was not observed in control somas (FIG. 17Ab, red) or the KO mutant neuronal somas (FIG. 17Bb). The paranodal staining of Cntnap1 and Contactin is robustly visible in FIGS. 17Aa, b, overlapped in FIG. 17Ad, and is absent in KO, C324R/− and R765C/− mutant neurons (FIGS. 17B-D). These data indicate that Cntnap1^{C324R} and Cntnap1^{R765C} mutant proteins are retained in the neuronal soma, most likely in the ER, and are unable to be properly transported out of the neuron, unlike the normal Cntnap1 protein, whose levels are very low within the neuronal soma but is clearly localized at the paranodal domains of myelinated axons.

The Cntnap1 protein at the paranodal axo-glial junctions on the axonal membrane side is believed to exist in a cis complex with the paranodal Contactin (schematic in FIG. 17E) forming the important axo-glial junctional complex between the axon and the myelin paranodal loops (Bhat M A, et al. Neuron. 2001; 30(2):369-383; Boyle M E, et al. Neuron. 2001; 30(2):385-397; and Pillai A M, et al. J Neurosci Res. 2009; 87(8):1773-1793). To determine if Cntnap1^{C24R} ad Cntnap1^{R765C} mutations had any deleterious effects on inter-protein interactions and protein-protein complex formation between Cntnap1 and Contactin, immunoprecipitations (IP) was carried out using membrane-enriched protein preparations from the spinal cords of +/−, C324R/−, R765C/−, TgEx;C324R/−, TgEx;R765C/− animals. As shown in FIG. 17F, the relative levels of Cntnap1 were low in C324R/− and R765C/− lysates compared to other genotypes. The Contactin protein levels were unaffected in C324R/− and R765C/− mutants, and the relative Contactin levels were similar to control +/− mice. The solubilized membrane preparations were immunoprecipitated with anti-Contactin antibodies followed by immunoblotting against Cntnap1. As shown in FIG. 17F, the levels of immunoprecipitated Cntnap1 were significantly low in C324R/− and R765C/− preparations compared to +/−, TgEx;C324R/−, TgEx;R765C/− preparations indicating that the binding between mutant Cntnap1^{C324R} and Cntnap1^{R76C} proteins and Contactin was severely affected. These IP data show that C324R and R765C amino acid changes affected protein-protein interactions between Cntnap1 and Contactin.

To further determine if Cntnap1^{C324R} and Cntnap1^{R765C/-} mutations had any impact on the surface expression of Cntnap1 and Contactin, human Cntnap1 expression construct [pCMV-Cntnap1^{Flag} (MR223061)] was obtained from OriGene and generated independent constructs of Cntnap1^{C324R} and Cntnap1^{R765C} mutations using site-directed mutagenesis (primers listed in Table1). After confirming the mutations, the wild-type Cntnap1, Cntnap1C^{C324R} or Cntnap1^{R765C/-} cDNAs and Contactin cDNA (MG50933-CF, SinoBiological) were co-transfected into HEK cells. Contactin surface expression was examined by immunostaining of live, non-permeabilized HEK cells followed by fixation and permeabilization and immunostaining with an anti-Cntnap1 antibody to detect the expression of Cntnap1. The wild-type Cntnap1 protein was readily localized at the cell surface of the HEK cells (FIGS. 17Ga, c), however, the surface localization of Cntnap1^{C324R} (FIGS. 17Ha, c), and Cntnap1^{R765}c (FIGS. 17Ia, c) mutant proteins was strongly affected. Similarly, the Contactin protein surface expression was robust in the presence of wild-type Cntnap1 as visualized by anti-Contactin/F3 immunostaining (FIGS. 17Gb, c, red) (Rios J C, et al. J Neurosci. 2000; 20(22):8354-8364); in HEK cells co-transfected with either Cntnap1^{C324R}(FIGS. 17Hb, c) or Cntnap1^{R765C} (FIGS. 17Ib, c) constructs, the surface expression of Contactin was dramatically reduced. The Cntnap1 protein expression was mostly cytosolic in both Cntnap1^{C324R} or Cntnap1^{R765C} transfected HEK cells as was the Contactin localization. These data strongly indicate that Cntnap1^{C324R} or Cntnap1^{R765C} mutant proteins are unable to co-transport with Contactin to cell surface and are mostly retained in the cytoplasm possibly ER, as is also observed in vivo in the spinal cord neurons (FIGS. 17C, D). IP of cell lysates from HEK cells that co-expressed the wild-type Cntnap1, Cntnap1^{C324R} and Cntnap1^{R765C} proteins and Contactin was also carried out. The expression levels of these proteins were similar in HEK cells transfected with respective expression constructs (FIG. 17J, input). As shown in FIG. 17J, co-IP using anti-Contactin antibodies, again showed reduced levels of Cntnap1 in Cntnap1^{C324R} (60% compared to Cntnap1) or Cntnap1^{R765C} (10% compared to Cntnap1) co-transfected HEK cells lysates further demonstrating that Cntnap1^{C324R} and Cntnap1^{R765C} mutant proteins fail to bind efficiently with Contactin compared to the wild-type Cntnap1.

TABLE 1
List of DNA primers for generating Cntnap1 mutant plasmid

| DNA mutant | Forward primers | Reverse Primers |
|---|---|---|
| Cntnap1^{C324} | GCCATAACTTCCGTGGCCGC | GTAGATCACGTTTTCTATGCGGC |
| | ATAGAAAACGTGATCTAC | CACGGAAGTTATGGC |
| | (SEQ ID NO: 13) | (SEQ ID NO: 14) |
| Cntnap1^{R765C} | GGTAGGTGATACAAACTGCT | GCTTCAGAATTTGAGCAGTTTGT |
| | CAAATTCTGAAGC | ATCACCTACC |
| | (SEQ ID NO: 15) | (SEQ ID NO: 16) |

*The underline indicates the codon for substituting amino acids.

Next, the surface biotinylation assay was carried out to quantitatively measure the surface expression of wild-type Cntnap1, Cntnap1^{C324R} or Cntnap1^{R765C} mutant proteins. First, as shown in FIG. 17K, surface expression of wild-type Cntnap1 and Cntnap1^{C324R} or Cntnap1^{R765C/-} mutant proteins was severely impaired when expressed without Contactin, however, Contactin was expressed at the cell surface in the absence of Cntnap1 (FIG. 17K). EGFR and GAPDH were used as surface and total input protein expression markers, respectively. These data demonstrate that in the absence of Contactin, the Cntnap1 protein is unable to get transported to the cell surface indicating that Contactin is important for cell surface expression of Cntnap1, which is consistent with previous in vivo studies in the Contactin null mutants (Boyle M E, et al. Neuron. 2001; 30(2):385-397). Next, the surface expression of Cntnap1 or Contactin, when the wild-type Cntnap1, Cntnap1^{C324R} or Cntnap1^{R765}c were transfected into HEK cells along with Contactin was examined (FIG. 17L). There is ˜80% reduction in Cntnap1^{C324R} surface protein levels and ˜90% reduction of surface protein expression for Cntnap1^{R765C} protein when compared to surface expression of the wild-type Cntnap1 protein (FIGS. 17L, M). Interestingly, the surface expression of Contactin was also reduced (˜50%) in Cntnap1^{C324R} and Cntnap1^{R765C/-} mutant co-expressed cells compared to wild-type Cntnap1/Contactin co-expression (FIGS. 17L, N). Together, these data demonstrate that Cntnap1³²⁴R and Cntnap1^{R765C} mutations disrupt the protein-protein interactions between Cntnap1 and Contactin, which also leads to defective co-transport and reduced cell surface expression of Cntnap1^{C324R} and Cntnap1^{R765C} mutant proteins.

Discussion Since late 1800s, a large spectrum of hereditary neuropathies have been identified, some of which display brain disorders with progressive muscle weakening (Tooth HH. The peroneal type of progressive muscular atrophy. University of Cambridge. 1886; Charcot J M. Rev Med Fr. 1886; 6:97-138; Scherer S S. Neuron. 2006; 51(6):672-674; Kramarz C, and Rossor A M. J Neurol. 2022; 269(9):5187-5191; and Frasquet M, and Sevilla T. Curr Opin Neurol. 2022; 35(5):562-570), and others display childhood hypomyelinating neuropathies (CHN), that have been linked to mutations in several genes associated with myelination (Scherer S S. Neuron. 2006; 51(6):672-674; Phillips J P, et al. Pediatr Neurol. 1999; 20(3):226-232; and Harati Y, and Butler I J. Journal of Neurology, Neurosurgery &amp; Psychiatry. 1985; 48(12):1269-1276). CNTNAP1 mutations in human diseases are associated with CHN. Four families were found to carry frameshift mutations in the CNTNAP1 gene that led to similar fetal phenotypes in the 7 offspring (Laquerriere A, et al. Hum Mol Genet. 2014; 23(9):2279-2289). With the rapid development of next-generation sequencing, close to cases around the globe have been identified from diverse backgrounds (American, Chinese, English, French, Irish and Palestinian). These patients present hypomyelination and hypotonia, consistent with the known phenotypes observed in Cntnap1 null mutant mice (Bhat M A, et al. Neuron. 2001; 30(2):369-383). Described herein are the generation and characterization of two mouse models each harboring a single nucleotide mutation in the mouse Cntnap1 gene, C324R/− and R765C/− representing two human CNTNAP1 mutations. The data reveal that either of these two mutations in Cntnap1 leads to instability of the Cntnap1 protein, disruption of the paranodal domains, reduction in nerve conduction velocity, and decline in motor coordination/function. Also described herein is an inducible wild-type Cntnap1 expression strain in the C324R/− and R765C/− mutant backgrounds, which led to Cntnap1 localization to the paranodal domains and re-established the paranodal axon/glial junctions. Physiologically, these mice re-gained proper nerve conduction and restored fine motor coordination/unction. These studies provide evidence that utilizing the wild-type copy of Cntnap1 to provide treatment options for human CNTNAP1 genetic deficiency-associated neurological disorders.

Paranodal Domains in C324R/− and R765C/− Mutant Myelinated Axons. The human CNTNAP1 protein (1384 amino acids, NP_003623) and the mouse Cntnap1 protein (1385 amino acids, 054991.2) are composed of a long extracellular domain, a transmembrane domain, and a relatively short intracellular domain (Bhat M A, et al. Neuron. 2001; 30(2):369-383; Peles E, et al. EMBO J 1997; 16(5):978-988; and Menegoz M, et al. Neuron. 1997; 19(2):319-331). The human CNTNAP1 mutation that changes Cys at position 323 to Arg (mouse C324R) is the most common CNTNAP1 mutation linked to congenital hypomyelinating neuropathy type 3 (CHN3, OMIM 618186). This missense variant, located in the first laminin G domain in the extracellular region, potentially causes a conformational change in the 3D structure of the Cntnap1 protein. This change is also expected to disrupt a potential disulfide bond between Cys324 and Cys355. The Arg764Cys is another mutation identified in the human CNTNAP1 gene, which also contributes to CHN3 (Vallat J M, et al. J Neuropathol Exp Neurol. 2016; 75(12):1155-1159). The generation of Cntnap1³²⁴⁴ and Cntnap1^{R765C} mutant mice as models of the human CNTNAP1 mutations allowed the characterization of the phenotypes and how these mutant proteins affected axonal domain organization and associated deficits. Both Cntnap1^{C324R} and Cntnap1^{R765C} mutant myelinated axons display disruption of the paranodal domains and loss of the barrier function of the paranodal region as observed by mislocalization of the juxtaparanodal complex into the paranodal areas next of the nodal complex, which is strictly separated in the wild-type myelinated axons (Bhat M A, et al. Neuron. 2001; 30(2):369-383).

Both Cntnap1^{C324R} and Cntnap1^{R765C} mutants showed similar but less severe phenotypes as compared to Cntnap1 knockout mice (Bhat et al., 2001). The human CNTNAP1 mutations have been associated with myelination defects mostly causing hypomyelination (Laquerriere A, et al. Hum Mol Genet. 2014; 23(9):2279-2289; Sabbagh S, et al. Case Rep Med. 2020; 2020:8795607; Vallat J M, et al. J Neuropathol Exp Neurol. 2016; 75(12):1155-1159; and Hengel H, et al. Neurol Genet. 2017; 3(2):e144). The EM analyses also revealed the disorganization of the paranodal junctional structure where the paranodal axo-glial junctional septa were not present. Despite the fact that some mutant Cntnap1 proteins are seen at the paranodes, no axo-glial septa were detected in the mutant paranodal regions suggesting that the mutant proteins are not capable of establishing the paranodal junctions (FIG. 13). The EM analysis also revealed hypomyelination in both C324R/− and R765C/− mutants. In many patients harboring CNTNAP1 gene mutation, the absence of septate junctions was often observed (Vallat J M, et al. J Neuropathol Exp Neurol. 2016; 75(12):1155-1159; Mehta P, et al. Muscle Nerve. 2017; 55(5):761-765; Conant A, et al. J Child Neurol. 2018; 33(10):642-650; Low K J, et al. Eur J Hum Genet. 2018; 26(6):796-807; and Hengel H, et al. Neurol Genet. 2017; 3(2):e144). Therefore, the humanized murine mutation models demonstrate that single amino acid changes in Cntnap1 cause structural abnormalities at the paranodal/nodal regions that are likely to be equivalent to the phenotypes observed in human patients with CNTNAP1 mutations.

Protein Stability and Transport Defects in C324R/− and R765C/− Mutants. Phenotypic analysis of C324R/− and R765C/− mutants revealed major defects in the paranodal domain organization even though the mutant proteins are expressed. The biochemical analyses revealed a marked reduction in the amount of C324R/− and R765C/− mutant proteins at the paranodes in the peripheral and central myelinated axons; and in the CNS these proteins were undetectable at the paranodes. Both mutant proteins are expressed but are relatively less stable than the wild-type Cntnap1 protein as revealed by immunoblot analysis. Cntnap1^{C324R} mutants showed nearly 70% reduction (CNS) and nearly 80% reduction (PNS) in Cntnap1^{C324R} levels while Cntnap1^{R765C} mutants showed about 40% reduction (CNS) and 70% reduction (PNS) in Cntnap1^{R765C/-} levels compared to wild-type Cntnap1 protein demonstrating that these mutations had specific effects on the Cntnap1 protein stability and the folding properties of these proteins due to distinct chemical features of the substituting amino acids. These observations show that the functional deficits and the transport mechanisms may be distinct between the PNS and CNS myelinated axons. The differences in total protein stability also reflected differences in detectable levels of Cntnap1 in the paranodal region: there was significantly more Cntnap1^{R765C} protein in paranodal regions than Cntnap1^{C324R} even though both mutants were likely dysfunctional as neither of them was able to establish functional paranodal domains as revealed by loss of the paranodal barrier function. Both mutant proteins were retained in the soma of motor neurons as well as the DRG neurons, unlike the wild-type Cntnap1, indicating that these mutant proteins fail to either get properly sorted through the protein transport system and/or are primarily retailed in the ER due to potential misfolding as a result of these amino acid substitutions.

Further in vitro biochemical analysis with co-expression of Cntnap1, Cntnap1^{C324R}, and Cntnap1^{R765C} with Contactin in human HEK cells revealed that the surface expression of the mutant proteins is compromised. Biotin-labeling and pull-down assays revealed that the surface expression of Contactin protein was barely detectable in HEK cells expressing Cntnap1^{R765C} and Contactin, while Cntnap1^{C324R} still maintained detectable surface expression levels of Contactin. Furthermore, the biochemical interactions between Contactin and Cntnap1^{C324R} and Contactin and Cntap1^{R765C} revealed slightly better binding between Contactin and Cntnap1^{R765C} compared to Cntnap1^{C324R} but both mutant proteins showed significantly reduced binding when compared to wild-type Cntnap1 and Contactin further underscoring that these mutations have a significant impact on the interactions between Cntnap1 and Contactin. Interestingly, another study on different Cntnap1 mutations showed no difference in the mRNA levels of Cntnap1^{+/+}, Cntnap1^{D1140Y} and Cntnap1^{G1251R} mutants from in-vitro primary neuronal cultures, but displayed variable protein stability between Cntnap1^{D1140Y} and Cntnap1^{G1251R} mutants, suggesting that each mutation has a distinct impact on the stability and/or folding of the Cntnap1 protein (Li W, et al. JCI Insight. 2020; 5(21)). An important question that remains to be addressed is the retention of Cntnap1^{C324R} and Cntnap1^{R765C/-} mutant proteins along with Contactin in the mutants. Although the biochemical interactions between Cntnap1^{C324R} and Cntnap1^{R761C} mutant proteins and Contactin are significantly reduced, there seem to be sufficient protein-protein interactions between these mutant proteins and Contactin to retain Contactin in the mutant neuronal somas (FIG. 17). In contrast, in the total absence of Cntnap1 in the Cntnap1 null mice (Bhat M A, et al. Neuron. 2001; 30(2):369-383), Contactin is not retained in the soma and gets transported out to the cell surface. On the other hand, in Contactin null mice Cntnap1 is retained in the neuronal soma indicating that Contactin is important for Cntnap1 transport to cell surface (Boyle M E, et al. Neuron. 2001; 30(2):385-397). Together, these findings on the Cntnap1^{C324R} and Cntnap1^{R765C} mutant proteins indicate that distinct amino acid substitutions in Cntnap1 lead to deleterious effects on protein folding, stability, and functionality, and that these changed properties have an impact on proper protein transport to the cell surface.

### Restoration of the Paranodal Domains and Motor Functions in Cntnap1 Mutants.

Life expectancy in patients with CNTNAP1 mutation, either with LCCS7 or CHN, is usually limited to death in the neonatal period, varying from a few hours of life to early childhood (Sabbagh S, et al. Case Rep Med. 2020; 2020:8795607; and Conant A, et al. J Child Neurol. 2018; 33(10):642-650). The lack of a direct link between specific mutations and varying levels of disease severity does not rule out the possibility that better medical treatment may be a factor in the improved survival of some individuals with the disease into their teenage years. There is no effective treatment for CNTNAP1 mutation-associated disease yet; however, early extensive medical care has recently proven to elongate life expectancy.

Therefore, there is an urgent need to examine any possible therapeutic options for this devastating disease. Most notably, the single amino acid changes in Cntnap1 do not seem to suggest any gain-of-function, or dominant adverse effects, as both Cntnap1^{C324R/+} and Cntnap1^{R765C/+} heterozygous mutants do not display any obvious abnormality compared to Cntnap1^{+/-} littermates. These observations led us to generate an inducible mouse strain in which the Cntnap1 expression was controlled by tamoxifen-dependent removal of the LoxP-Stop-LoxP sequences (Kim H, et al. Lab Anim Res. 2018; 34(4):147-159; and Sharma S, and Zhu J. Curr Protoc Immunol. 2014; 105:10 34 11-10 34 13). As the induced Cntnap1^{Flag} protein properly localized at the paranodes and was indistinguishable from the wild-type Cntnap1, its expression in C324R/− and R765C/− mutants allowed re-organization of the paranodes and also significant restoration of the motor functions. Most of the phenotypes observed in C324R/− and R765C/− mutants were significantly restored including the reorganization of the paranodes, re-separation of the axonal domains, the formation of the axo-glial septa at the paranodal junctions and most importantly the nerve conduction properties. Most remarkably, the outer paranodal loops that were often seen everted at the paranodes could re-establish axo-glial septa with the axonal axolemma as revealed by EM analysis indicating that expression of Cntnap1 allows the progressive re-establishment of the paranodal axo-glial junctions. The kinetics of expression of Cntnap1 after induction and restoration of the paranodal domains certainly revealed a timeline in which the Cntnap1 protein can transport through myelinated axons and localize to the paranodes. The re-establishment of interactions with Contactin and glial NF155 may also be a timely process as the phenotypic rescue gets better with time and within 6-7 weeks there was a marked improvement in the phenotypic rescue and restoration. Of significant importance is the observation that the body weight of C324R/− and R765C/− mutants showed no marked improvement despite significant restoration of motor functions. Like KO mutants (Bhat M A, et al. Neuron. 2001; 30(2):369-383; and Pillai A M, et al. J Neurosci Res. 2007; 85(11):2318-2331), both C324R/− and R765C/− suffered significant reduction in body weight as compared to wild-type or Cntnap1^{+/-} mice due to severe muscle atrophy. Since tamoxifen injections were carried out at 3 weeks postnatally, it is likely that some muscle atrophy had already occurred or loss of some neuromuscular functions, as has been previously reported for Cntnap1 and Cntnap2 mutants (Saifetiarova J. et al. J Neurosci Res. 2017; 95(7):1373-1390). These observations underscore that timely expression of Cntnap1 in C324R/− and R765C/− or other loss of function Cntnap1 mutants will be able to restore the deficits that are caused by mutant Cntnap1 proteins.

Relevance to Human Disease and Therapies. A number of mouse and human studies related to axonal domain disorganization have been linked to various neurological diseases with auditory, motor, and nerve conduction impairments. Described herein are mouse models carrying the human CNTNAP1 mutations, which can be utilized for clinical studies. These humanized Cntnap1 mouse models showed similar detrimental changes in the structure and function of the axonal domains, affecting the electrical and physiological properties of myelinated axons in both the CNS and PNS, and resulting in severe ataxia and paralysis. The results demonstrate that restoring the functional nodal organization in Cntnap1 mouse models is possible by re-expressing the wild-type Cntnap1 protein. These rescue strategies could be used to determine the time course of reversing pathologies in human patients carrying CNTNAP1 mutations. These mouse studies can be used to further human studies using viral vector-based strategies with gene therapy. Taken together, the strategies disclosed herein can serve to alleviate the consequences of human CNTNAP1 mutations-related neurological diseases, and to help slow down or cure the genetic diseases related to myelinated axon pathologies.

Materials and Methods. Generation of Cntnap1 Mouse Mutants and LoxP-Stop-LoxP-Cntnap1 Transgenic Mice. The transgenic mice described herein, except the ones purchased from Jackson laboratory, were generated in the Mouse Genome Engineering and Transgenic Facility at UT Health Science Center in San Antonio. Cntnap1 knockout mice have been reported previously (Bhat M A, et al. Neuron. 2001; 30(2):369-383). To introduce the Cysteine to Arginine (C324R) and Arginine 765 to Cysteine (R765C) mutations in the mouse Cntnap1 single guide (sg) RNAs 5′-GCCTACCGCCATAACTTCCG-3′ (SEQ ID NO: 1) (for C324R) or 5′-CTCAAATTCTGAAGCTCAGT-3′ (SEQ ID NO: 2) (for R765C) were selected with minimum off-target effects (designed by CRISPOR). 198 base pairs (bp) ssDNA donors with sequences changing TGC>CGC (C324R) or CGC>TGC (R765C) were used along with guide RNAs and Cas9 protein complex for pronuclear microinjection (the gRNA and donor DNA sequences were listed in Table 2). The pups were genotyped and screened by Sanger sequencing (Eurofins, Tc). The entire Cntnap1 cDNAs from each mouse mutant were fully sequenced to ensure no additional mutation/s were created in the Cntnap1 gene.

TABLE 2
List of DNA sequences for generating the transgenic mutant mice.

| Mice line | gRNA | Donor DNA |
|---|---|---|
| Cntnap1^{C324R/-} | GCCUACC | CCTAAGCCCATGGAAATGTCTCTTCATCTGCTTCCT |
| | GCCAUAA | ACAGATATTCATCGGGGGTCTAGTGGGCGCAGCCC |
| | UUUUCG | GTAAGAACCTGGCCTACCGCCATAATTTTCGCGGC |
| | (SEQ ID | CGCATAGAAAACGTGATCTACAACCGGATCAACAT |
| | NO: 17) | TGCAGAAATGGCAGTGATGCGCCATTCGCGGATCA |
| | | CCTTTGAGGCCAGTGGGCAGGG |
| | | (SEQ ID NO: 18) |
| Cntnap1^{R765C/-} | CUCAAAU | GAGTCACCGAATCCTATATTCCCACAGGAGAACAG |
| | UCUGAAG | ACAAGGGGCTCCTGACCTTTGTAGACCATCTGCCT |
| | CUCAGU | GTCACTCAGGTAGTGGTAGGTGATACAAACIGCTC |
| | (SEQ ID | AAATTCTGAAGCTCAGTTCTTCCTGAGGCCTCTGCG |
| | NO: 19) | CTGCTATGGTGACCGTGAGTGGCAGACTCCTTTGG |
| | | TGTGCCTGTCCAGTACTGTTTC |
| | | (SEQ ID NO: 20) |
| LSL- | ACUCCAG | Linearized LSL-GAG-Cntnap1^{Flag} construct |
| Cntnap1^{Flag} | UCUUUCU | |
| | AGAAGA | |
| | (SEQ ID | |
| | NO: 21) | |

To generate an inducible tissue-specific overexpression of wild-type Cntnap1, LoxP-Stop-LoxP-Cntnap1^{Flag} was first generated. This construct was linearized with the NheI enzyme and micro-injected into pronuclei. The offspring were screened by using the primers in the Cntnap1 locus that differentiated the endogenous Cntnap1 from the transgenic Cntnap1. This mouse strain is referred to as LSL-Cntnap1. LSL-Cntnap1 was then crossed with SLICK-H-CreERT2 (JAX:012708) to generate SLICK-H-CreERT2;LSL-Cntnap1 mice. To enable the expression of the Cntnap1 transgene in neurons, tamoxifen (MP Biomedicals) at 1 mg/μL was delivered by intraperitoneal (i.p.) injections for 5 consecutive days at postnatal (P)-21 at a dose of 1 mg/12.5 g body weight. At the time of experiments, age-matched control, SLICK-H-CreERT2;LSL-Cntnap1, C324R/− and R765C/− and a combination of appropriate genotype littermates were evaluated by electrophysiological, immunohistochemical, biochemical, and ultrastructural techniques.

DNA Constructs and Cell Transfections. Wild-type mouse Cntnap1 cDNA plasmid was purchased from OriGene (MR223061); and Contactin cDNA cloning plasmid was obtained from SinoBiological (China) (MG50933-CF). Cntnap1C324R or R765C mutant forms were generated by using a QuikChange site-directed mutagenesis kit (Stratagene, CA). The DNA primers used are listed in Table 1. The presence of T>C (for C324R) or C>T (for R765C) mutations in Cntnap1 and the full Cntnap1 cDNA were verified by full coding sequence sequencing (Eurofins Genomics LLC). HEK cells for Cntnap1 expression were obtained from ATCC and maintained in a humid 5% CO₂, 37° C. incubator with high glucose DMEM containing 10% fetal bovine serum (FBS) and antibiotics. DNA constructs were transiently transfected into HEK cells using Lipofectamine 2000 (Invitrogen) 24 h after plating of the cells on 60-mm dishes (collecting cells for western blotting) or 4-chamber slides (for immunostaining). Then, the cells were ready for downstream experiments 48-72 hours after transfection (Shi Q, et al. J Neurosci. 2022; 42(27):5294-5313).

Antibodies and other Reagents. Chemicals and reagents were purchased from Sigma-Aldrich unless otherwise specified. The antibodies used in this study were listed in Table3. Infrared (IR) conjugated secondary antibodies used for immunoblotting were from LI-COR. Alexa Fluor 488 (B13422) and 594 (B13423) conjugated-secondary antibodies for immunostaining (Alexa Fluor-488, 568, 647) were purchased from Invitrogen (USA).

TABLE 3
List of Reagents

| | | Identifier or |
|---|---|---|
| | Resource | Reference |
| Antibodies | | |
| Rabbit anti-Cntnap1 | Bhat Laboratory | (Bhat et al., 2001) |
| Guinea Pig anti-Cntnap1 | Bhat Laboratory | (Bhat et al., 2001) |
| Rat anti- NFCT | Bhat Laboratory | (Pillai et al., 2009) |
| Guinea Pig anti- NF186 | Bhat Laboratory | (Thaxton et al., |
| | | 2011; Thaxton et |
| | | al., 2010) |
| Rabbit anti-Neurofascin155 | Cell Signaling | #15035 |
| Rabbit anti-AnkG | Bhat Laboratory | (Saifetiarova et al., |
| | | 2017) |
| Rabbit anti- βIV Spectrin | Bhat Laboratory | (Saifetiarova et al., |
| | | 2018) |
| Mouse anti- pan NaV Channels | Sigma | S8809 |
| Mouse anti- KV1.2 | NeuroMab | K14/16 |
| Mouse anti-NeuN | Millipore-Sigma | MAB377 |
| Mouse anti-b-actin | Sigma | A2228 |
| Rabbit anti-Flag | Cell Signaling | #14793 |
| Mouse anti-Flag | Sigma | F-1804 |
| DNA constructs | | |
| Mouse Cntnap1 ORF clone | Origene, USA | MR223061 |
| Mouse Cntnap1^{C324R} clone | Bhat Laboratory | This study |
| Mouse Cntn1 ORF clone | SinoBiological, | MG50933-CF |
| | China | |
| Mouse Cntnap1^{R765C} clone | Bhat Laboratory | This study |
| LSL-GAG-Cntnap1^{Flag} | Bhat Laboratory | This study |
| Mouse lines | | |
| Cntnap1 KO mice | Bhat Laboratory | Bhat et al., 2001 |
| Cntnap1^{C324R} mice | Bhat Laboratory | This study |
| Cntnap1^{R765C} mice | Bhat Laboratory | This study |
| Slick-H-Cre | Jackson | Jax#012708 |
| | Laboratory | |
| LSL-Cntnap1^{Flag} | Bhat Laboratory | This study |
| Commercial kits | | |
| Cell Surface Protein | Pierce ™ at | # 89881 |
| Isolation Kit | ThemoFisher | |

Rotarod Test. Motor function performance was assessed using the Rotarod apparatus (Ugo Basile) (Steele et al., 1998). At least 6 mice per genotype and both male and female mice were included for motor performance studies. Mice were trained on the Rotarod apparatus at 5 RPM for 5 min for three consecutive days. For testing, the speed was gradually accelerated to 25 RPM or 35 RPM over 2 minutes. Latency to fall was recorded for each trial. Each mouse went through three trials. Results are shown as a mean of the three trials ±SEM.

Beam-Walking Test. This test was used for the assessment of fine motor coordination, particularly of the hindlimb. Firstly, animals were placed on a round beam and allowed to walk across the beam from one end to the other for at least three times. The beam measures 1.5 cm in diameter and is secured on an open tray. This training step can be useful to achieve a stable baseline measurement. The time taken to cross the beam was recorded for each trial.

Tissue Preparation and Immunostaining. Animals were anesthetized with i.p. injection of Avertin (400 mg/kg mouse body weight) and transcardially perfused with phosphate-buffered saline (PBS) (pH 7.2-7.4). The cervical region of the spinal cord (SC) was dissected and postfixed in 4% paraformaldehyde (PFA) overnight at 4° C. and then immersed in 30% sucrose in 0.1M phosphate buffer until they settled to the bottom. The tissue was rinsed several times in PBS and frozen in Tissue-Tek O.C.T. Compound (Sakura Finetek USA, Inc). Longitudinal 20 μm sections for SCs were cut with a cryostat (Leica), mounted on slides, and processed for immunostaining. For the peripheral nerves, proximal sciatic nerves (SNs) were dissected out from anesthetized animals, fixed in 4% PFA for 30 minutes, washed in PBS and teased into individual nerve fibers, dried overnight at room temperature, and stored at −80° C. Immunostaining of the samples was carried out as previously described (Bhat et al., 2001; Shi et al., 2019, Saifetiarova et al., 2018).

Chemical Labeling of Surface Proteins in HEK Cells. The expression of plasma membrane proteins on HEK cells was analyzed with cell surface protein extraction kit. Briefly, Sulfo-NHS-SS-Biotin was first dissolved in 0.1% DMSO in PBS. Forty-eight hours after transfection, HEK cells were washed three times with PBS (with Ca²⁺ and Mg²⁺) and then treated with 2 mM Sulfo-NHS-SS-Biotin/PBS for 30 min at 4° C. The cells were then collected in chilled lysis buffer containing protease inhibitors and kept on ice for 1 hour. The total protein concentration was determined by MicroBCA kit (Pierce), and equal amounts of total protein were mixed with Streptavidin beads for 1 hr at room temperature. Biotinylated proteins were eluted from Streptavidin beads using 100 μl of eluate buffer after 3× washes with washing buffer. The eluted sample was immunoblotted by anti-Cntnap1, and anti-Contactin antibodies. Anti-EGFP or GAPDH were served as a surface protein marker or loading control.

Image Analysis. Confocal images were acquired with a Zeiss LSM 710 Microscope (Saifetiarova J, et al. J Neurosci. 2018; 38(28):6267-6282; and Taylor A M, et al. Front Cell Neurosci. 2017; 11:11). Briefly, identical settings were used to capture images from the control and the mutant samples, and the images shown are maximal-intensity projections from 5-8 Z-stacks with 0.25-μm intervals. For the quantification of nodal intensities, z-stack images were used to quantify intensities for each genotype, and a minimum of 50 nodes for each nodal marker per sample were quantified.

Immunoblotting. SCs and SNs were harvested and stored at −80° C. until processed. Tissues were homogenized using glass mortar and pestle in ice-cold lysis buffer with protease inhibitors (#A32953, Thermo Scientific). The lysates were incubated for 30 min on ice and then centrifuged at 10,000×g for 30 min at 4° C. Supernatants were collected, and protein concentrations were estimated using a Pierce BCA protein assay Kit (Thermo Scientific). Equal amounts of protein were resolved by 4-12% SDS-PAGE and transferred onto nitrocellulose membranes. The membranes were blocked in 10% non-fat dry milk for 1 hour followed by incubation with appropriate primary antibodies in TBST overnight at 4° C. After three-time washes in TBST, membranes were incubated with IR-conjugated secondary antibodies for 1 hour and scanned using Li-COR Odyssey infrared imager. The intensities of immunoblot bands were quantified using ImageJ software (RRID:SCR_003070; NIH) and normalized to D-Actin as the loading control.

In vivo Nerve Conduction Recordings. Animals were anesthetized by continuous isoflurane (5% aerosolized) for in vivo Nerve Conduction Velocity (NCV) recordings using a Nicolet Teca Synergy system (Natus Neurology Inc., Middleton, WI). In order to maintain body temperature to 33-34° C. during recordings, a warming lamp was kept above mice. NCVs and amplitudes were recorded from the tail and SN of anesthetized mice (Saifetiarova J, et al. J Neurosci Res. 2017; 95(7):1373-1390; and Taylor A M, et al. Front Cell Neurosci. 2017; 11:11). For SN recording, two separate recordings were made in the dorsum of the foot with a stimulus first given at the ankle (0.02 ms, 2 mA) and secondly at the sciatic notch (0.02 ms, 8 mA). Once traces were acquired, NCV was calculated by the distance divided by the latency and amplitude was measured as the height of the peak. For sciatic NCV, the distance between the notch and ankle was divided by the latency between the notch and ankle.

Transmission Electron Microscopy. For transmission electron microscopy (TEM), the tissue were processed (Saifetiarova J, et al. J Neurosci Res. 2017; 95(7):1373-1390; and Green J A, et al. BMC Neurosci. 2013; 14:96). The solutions were freshly prepared on the day of perfusion. In brief, animals were anesthetized and transcardially perfused with normal saline followed by 2.5% glutaraldehyde/4% paraformaldehyde EM fixative (dissolved in 0.16 M NaH₂PO4/0.11M NaOH buffer, pH 7.2-7.4) for 30 mins. After perfusion, entire mouse carcasses were post-fixed for at least 1 week in the same EM fixative. SNs, SCs were dissected out and incubated overnight in 0. IM sodium cacodylate buffer followed by incubation in 2% OSO₄ solution and gradient ethanol dehydration. Samples were incubated in propylene oxide, left in 100% PolyBed resin for 36 hours and embedded in flat molds at 55° C. for 36 hours. After embedding, the molds were processed and imaged on a JEOL 1230 electron microscope at the UTHSCSA Electron Microscopy Lab. TEM Images were analyzed by Image J software (NIH).

g-Ratios of Myelinated Axons. The g-ratios of SNs and SCs myelinated fibers from TEM images were measured by MyelTracer software (Kaiser T, et al. eNeuro. 2021; 8(4)). At least 20 images per animal taken at 5600× magnification were used for the g-ratio measurement using at least 150 axons in 3 independent mice for each genotype. The g-ratio was measured as the ratio of the inner axonal radius to the outer fiber radius with the myelin sheath.

Co-ImmunoprecipitationAssay. Mouse brains were homogenized in IP lysis buffer (50 mM Tris-HCl pH 7.4, 150 mM NaCl, 1% NP-40, 1 mM EDTA, 5% glycerol with proteinase inhibitors) (Garcia-Fresco G P, et al. Proc Natl Acad Sci USA. 2006; 103(13):5137-5142). Nuclei and cell debris were removed by low-speed centrifugation (3,000×g for 10 min at 4° C.). The supernatants were centrifuged at 400,000×g for 60 min at 4° C. to obtain the microsomal membrane protein fraction. The membrane pellets were solubilized in Triton X-100 lysis buffer (50 mM Tris-HCl, pH 7.4, 150 mM NaCl, 1% triton x-100, 5 mM EDTA). After clearing at 10,000×g for 15 min, the membrane fraction supernatants were incubated with desired antibody and Protein A/G beads (Santa Cruz, sc-2003) overnight mixing at 4° C. The beads were washed three times with Trion X-100 lysis buffer the next day, and the binding proteins were eluted with 2×SDS PAGE sample buffer. The membrane input and eluted fractions were subjected to immunoblotting assays.

For in vitro cell experiments, total cell lysates were prepared in IP lysis buffer as described herein and cleaned at 13,000×g for 30 min at 4° C. Then, the supernatant was incubated with desired antibody and Protein A/G beads overnight and processed as described for brain tissues.

Statistical Analysis. For immunostaining, tissues were processed from 3 mice per group per time point, then 50-70 nodes from PNS and CNS were quantified per animal. For immunoblotting, tissues were processed and quantified from 3 mice per group at the terminal time point. For ultrastructure analyses, tissues were processed from 3 mice per group, and for each mouse, a minimum of 200 axons for SN and SC were imaged. Data measurements and analysis were performed by one examiner in a non-blinded manner. The data are presented as mean±S.E.M. N represents the number of animals (unless otherwise stated statistically significant differences between control and mutant groups were determined by 1- or 2-way ANOVA with Bonferroni's post hoc test using GraphPad Prism software and are represented by *P<0.05; **P<0.01; ***P<0.001.

What is claimed is:

1. A transgenic mouse comprising a genome, wherein the genome comprises a null mutation in a first copy of the mouse Contactin-associated protein 1 (Cntnap1) gene, and a mutation in a second copy of the mouse Cntnap1 gene, wherein the mutation in the second copy of the mouse Cntanp1 gene is T>C (for C324R), C>T (for R765C) or G>T (for G350V), and is expressed in the transgenic mouse.

2. The transgenic mouse of claim 1, wherein the null mutation is a deletion of mouse exon 7 and exon 8.

3. A transgenic mouse comprising a genome comprising a modified mouse Contactin-associated protein 1 (Cntnap1) gene, wherein the modified mouse Cntnap1 gene comprises a nucleotide modification compared to a wild-type mouse Cntnap1 gene, wherein the nucleic acid modification is a substitution of a thymine for a cytosine at nucleic acid position 4958 (for C324R) of a wild-type Cntnap1 gene of SEQ ID NO: 33, a substitution of a cytosine for thymine (for R765C) at nucleic acid position 8926 of a wild-type Cntnap1 gene of SEQ ID NO: 33, or substitution of a guanine for a thymine (for G350V) at nucleic acid position 5709 of a wild-type CNTNAP1 gene of SEQ ID NO: 33.

4. A transgenic mouse comprising a genome capable of expressing a modified Contactin-associated protein 1 (CNTNAP1) polypeptide, wherein the modified CNTNAP1 polypeptide comprises an amino acid modification compared to a wild-type CNTNAP1 polypeptide, wherein the amino acid modification is a substitution of a cysteine for a arginine at amino acid position 324 (for C324R) of a wild-type CNTNAP1 polypeptide of Accession ID NP_058062.2, a substitution of a arginine for a cysteine at amino acid position 765 (for R765C) of a wild-type CNTNAP1 polypeptide of Accession ID NP_058062.2, or a substitution of a glycine for a valine at amino acid position 350 (for G350V) of a wild-type CNTNAP1 polypeptide of Accession ID NP_058062.2.

5. The transgenic mouse of claim 3, wherein the transgenic mouse further comprises a wild-type inducible Contactin-associated protein 1 (Cntnap1) gene of SEQ ID NO: 33.

6. The transgenic mouse of claim 4, wherein the transgenic mouse further comprises a wild-type Contactin-associated protein 1 (CNTNAP1) polypeptide of SEQ ID NO: 25.

7. The transgenic mouse of any of claims 1-6, wherein the transgenic mouse displays hypomyelination, an increased g-ratio, or a combination thereof.

8. The transgenic mouse of any of claims 1-6, wherein the transgenic mouse displays weight loss, reduced nerve conduction, progressive motor dysfunction, severe ataxia, paralysis or a combination thereof associated with paranodal axonal domain disorganization or CNTNAP1-associated congenital hypomyelinating neuropathy.

9. A cell derived from the transgenic mouse of any of claims 1-8.

10. The cell of claim 9, wherein the cell is a neuron.

11. An embryo that is an offspring of the transgenic mouse of any of claims 1-8, wherein the embryo is heterozygous for the modified Contactin-associated protein 1 (CNTNAP1) gene or modified Contactin-associated protein 1 (CNTNAP1) polypeptide.

12. A polynucleotide comprising an expression cassette, wherein the expression cassette comprises a transcriptional regulatory region comprising a promoter operatively linked to a nucleotide sequence as set forth in SEQ ID NO: 24 encoding the human Contactin-associated protein 1 (hCNTNAP1) protein.

13. The polynucleotide of claim 12, wherein the promoter is a constitutive promoter.

14. The polynucleotide of claim 13, wherein the promoter is the human neuron promoter (hSyn) promoter.

15. The polynucleotide of claim 14, wherein the human neuron promoter has a nucleotide sequence of SEQ ID NO: 29.

16. The polynucleotide of claim 12, further comprising a Flag/Myc dual tail.

17. The polynucleotide of claim 16, wherein the Flag/Myc dual tail has a nucleotide sequence of SEQ ID NO: 30.

18. A vector comprising the polynucleotide of any of claims 12-17, wherein the vector is a lentiviral vector.

19. A vector comprising the polynucleotide of any of claims 12-17, wherein the vector is an adeno-associated viral vector.

20. The vector of claim 19, wherein the vector is an adeno-associated viral vector of serotype 9 (AAV9).

21. A pharmaceutical composition comprising a therapeutically effective amount of the vector of any of claims 19-20, and a pharmaceutically acceptable carrier and/or adjuvant.

22. A method of treating and/or preventing Contactin-associated protein 1 (hCNTNAP1) protein deficiency in a subject in need thereof, the method comprising administering to the subject, the pharmaceutical composition of claim 21.

23. A method of preventing or reducing severe respiratory distress in a subject in need thereof, the method comprising administering to the subject the pharmaceutical composition of claim 21.

24. A method of increasing nerve conduction in a subject in need thereof, the method comprising administering to the subject the pharmaceutical composition of claim 21.

25. A method of reducing ataxia in a subject in need thereof, the method comprising administering to the subject the pharmaceutical composition of claim 21.

26. A method of reducing motor dysfunction in a subject in need thereof, the method comprising administering to the subject the pharmaceutical composition of claim 21.

27. A method of reducing or reversing paralysis in a subject in need thereof, the method comprising administering to the subject the pharmaceutical composition of claim 21.

28. A method of ameliorating a symptom of a CNTNAP1 mutation in subject in need thereof, the method comprising administering to the subject the pharmaceutical composition of claim 21.

29. The method of claim 28, wherein the symptom of a CNTNAP1 mutation is polyhydramnios, severe neonatal hypotonia, arthrogryposis, severe motor paralysis, acute respiratory distress or muscle atrophy.

30. A method of treating a subject with a neuropathy caused by a CNTNAP1 mutation, the method comprising administering to the subject, the pharmaceutical composition of claim 23.

31. The method of any of claims 22-30, wherein the subject is a human.

32. The method of claim 32, wherein the human subject is an infant or a child.

33. A method for screening a test substance for treating a Contactin-associated protein 1 (hCNTNAP1) protein deficiency, comprising:
a) administering a test substance to the transgenic mouse of any of claims 1-5, and
b) determining the effect of the test substance on the at least one symptom of hCNTNAP1 protein deficiency, wherein a decrease in the at least one symptom of hCNTNAP1 protein deficiency as compared to a control indicates the test substance treats the hCNTNAP1 protein deficiency.

34. The method of claim 33, wherein the at least one symptoms of hCNTNAP1 protein deficiency is weight loss, reduced nerve conduction, progressive motor dysfunction, severe ataxia, paralysis or a combination thereof associated with paranodal axonal domain disorganization or CNTNAP1-associated congenital hypomyelinating neuropathy.

35. The method of claim 33, wherein the at least one symptoms of hCNTNAP1 protein deficiency is polyhydramnios, severe neonatal hypotonia, arthrogryposis, severe motor paralysis, acute respiratory distress or muscle atrophy

36. A method for screening a test substance for treating respiratory distress, the method comprising:
a) administering a test substance to the transgenic mouse of any of claims 1-5, and
b) determining the effect of the test substance on respiratory distress, wherein a decrease in the at least one symptom associated with respiratory distress as compared to a control indicates the test substance treats respiratory distress.

37. A method for screening a test substance for increasing nerve conduction, the method comprising:
a) administering a test substance to the transgenic mouse of any of claims 1-5, and
b) determining the effect of the test substance on nerve conduction, wherein the effect on nerve conduction as compared to a control indicates the test substance increases nerve conduction.

38. A method for screening a test substance for reducing ataxia, the method comprising:
a) administering a test substance to the transgenic mouse of any of claims 1-5, and
b) determining the effect of the test substance on ataxia, wherein the effect on ataxia as compared to a control indicates the test substance reduces ataxia.

39. A method for screening a test substance for reducing motor dysfunction, the method comprising:
a) administering a test substance to the transgenic mouse of any of claims 1-5, and
b) determining the effect of the test substance on motor dysfunction, wherein the effect on motor dysfunction as compared to a control indicates the test substance reduces motor dysfunction.

40. A method for screening a test substance for reducing or reversing paralysis, the method comprising:
a) administering a test substance to the transgenic mouse of any of claims 1-5, and
b) determining the effect of the test substance on paralysis, wherein the effect on paralysis as compared to a control indicates the test substance reduces or reverses paralysis.
